(19) United States
(12) Patent Application Publication
(10) Pub. No.: US 2023/0129941 A1
(43) Pub. Date: Apr. 27, 2023

(54) LIGHT EMITTING ELEMENT AND AMINE COMPOUND FOR THE SAME

(71) Applicant: Samsung Display Co., Ltd., Yongin-si (KR)

(72) Inventors: Ichiro IMADA, Yokohama (JP); Masatsugu UENO, Yokohama (JP)

(73) Assignee: Samsung Display Co., Ltd., Yongin-si (KR)

(21) Appl. No.: 17/945,491

(22) Filed: Sep. 15, 2022

(30) Foreign Application Priority Data
Oct. 25, 2021 (KR) 10-2021-0143043

(51) Int. Cl. H01L 51/00; C07D 409/12; C07D 307/91

(52) U.S. Cl. CPC H01L 51/0061; H01L 51/006; C07D 409/12; C07D 307/91; H01L 51/5064

(57) ABSTRACT

A light-emitting element includes a first electrode, a second electrode disposed on the first electrode, and at least one functional layer disposed between the first electrode and the second electrode and including an amine compound represented by Formula 1. The light-emitting element may exhibit high efficiency and long lifetime characteristics.

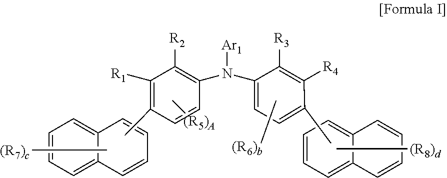

### CROSS REFERENCE TO RELATED APPLICATION(S)

This application claims priority to and benefits of Korean Patent Application No. 10-2021-0143043 under 35 U.S.C. § 119, filed on Oct. 25, 2021 in the Korean Intellectual Property Office, the entire contents of which are incorporated herein by reference.

### BACKGROUND

### 1. Technical Field

The disclosure relates to a light-emitting element including a novel amine compound in a hole transport region.

### 2. Description of the Related Art

Active development continues for an organic electroluminescence display device or the like as an image display device has. The organic electroluminescence display device or the like is a display device including a so-called self-luminous display element in which holes and electrons respectively injected from a first electrode and a second electrode recombine in a light-emitting layer so that a light-emitting material in the light-emitting layer emits light to achieve display.

In the application of a light-emitting element to a display device, there is a requirement for a low driving voltage, high luminous efficiency, and a long lifetime, and there is a demand for continuous development of a material for a light-emitting element that is capable of stably achieving such characteristics.

In order to implement a high-efficiency light-emitting element, there is also a demand for development of a material for a hole transport region for suppressing diffusion of exciton energy in a light-emitting layer.

It is to be understood that this background of the technology section is, in part, intended to provide useful background for understanding the technology. However, this background of the technology section may also include ideas, concepts, or recognitions that were not part of what was known or appreciated by those skilled in the pertinent art prior to a corresponding effective filing date of the subject matter disclosed herein.

### SUMMARY

The disclosure provides a light-emitting element exhibiting excellent luminous efficiency and long-life characteristics.

The disclosure also provides an amine compound, which is a material for a light-emitting element having high efficiency and a long lifetime.

An embodiment provides an amine compound which may be represented by Formula 1:

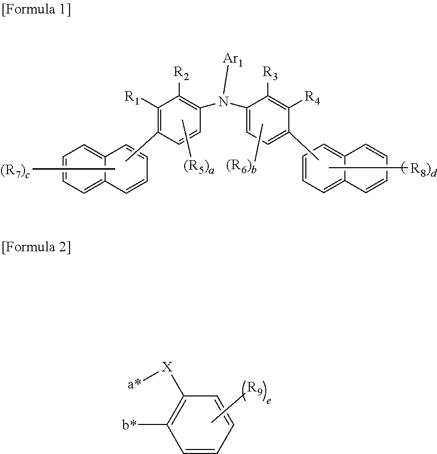

In Formula 1, Ar₁ may be a substituted or unsubstituted aryl group having 6 to 40 ring-forming carbon atoms other than a phenanthryl group, or a substituted or unsubstituted heteroaryl group having 5 to 40 ring-forming carbon atoms; R₁ to R₄ may each independently be a hydrogen atom, a deuterium atom, a substituted or unsubstituted alkyl group having 1 to 20 carbon atoms, a substituted or unsubstituted aryl group having 6 to 40 ring-forming carbon atoms, or may be a single bond forming a ring by bonding with a group represented by Formula 2; and a pair of R₁ and R₂ and/or a pair of R₃ and R₄ may be bonded with a group represented by Formula 2 to form a ring. In Formula 2, X may be O, S, N(R₁₀), or C(R₁₁)(R₁₂), and a* and b* each indicate a bond to any one of R₁ to R₄ in Formula 1. In Formula 1 and Formula 2, R₅ to R₁₂ may each independently be a hydrogen atom, a deuterium atom, a substituted or unsubstituted alkyl group having 1 to 20 carbon atoms, or a substituted or unsubstituted aryl group having 6 to 40 ring-forming carbon atoms; a and b may each independently be an integer from 0 to 2; c and d may each independently be an integer from 0 to 7; and e may be an integer from 0 to 4.

In an embodiment, the amine compound represented by Formula 1 may be represented by any one of Formulas 1-1 to 1-3:

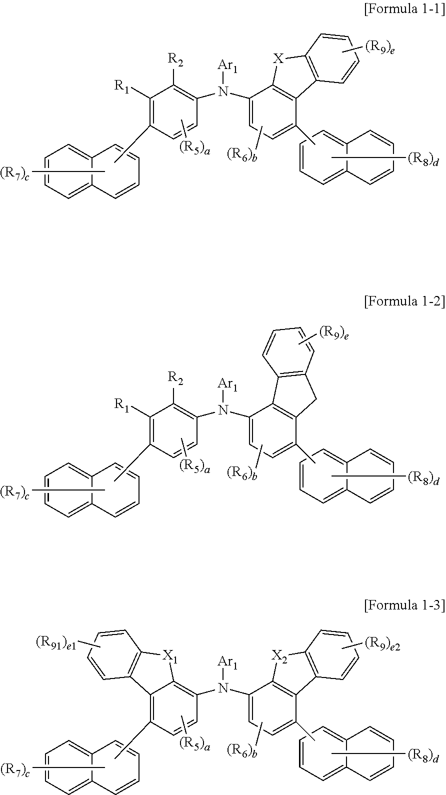

In Formula 1-3, X₁ and X₂ may each independently be O, S, N(R₁₀), or C(R₁₁)(R₁₂); e1 and e2 may each independently be an integer from 0 to 4; and R₉₁ and R₉₂ may each independently be a hydrogen atom, a deuterium atom, a substituted or unsubstituted alkyl group having 1 to 20 carbon atoms, or a substituted or unsubstituted aryl group having 6 to 40 ring-forming carbon atoms. In Formulas 1-1 to 1-3, Ar₁, X, R₁ to R₁₂, and a to e are the same as defined in Formulas 1 and 2.

In an embodiment, in Formula 1-3, X₁ and X₂ may be the same.

In an embodiment, the group represented by Formula 2 may be represented by any one of Formulas 2-1 to 2-5:

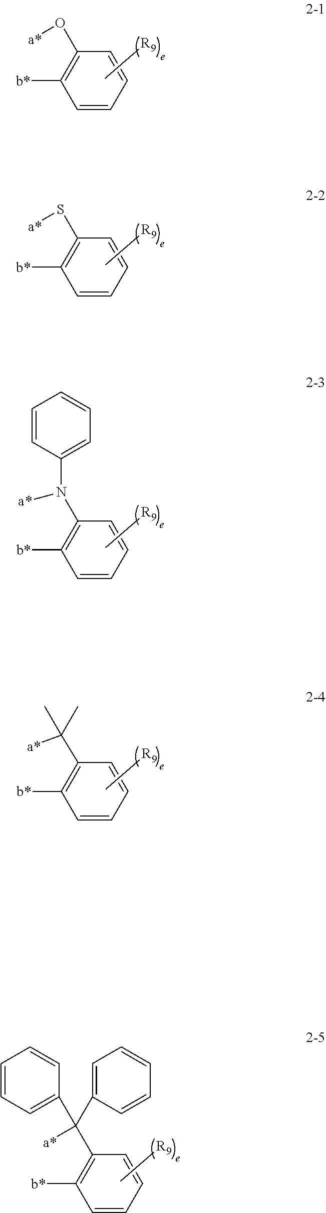

In formulas 2-1 to 2-5, R₉, e, a*, and b* in 2-1 to 2-5 are the same as defined in Formula 2.

In an embodiment, in Formula 1, Ar₁ may be a substituted or unsubstituted phenyl group, a substituted or unsubstituted naphthyl group, a substituted or unsubstituted dibenzothiophene group, or a substituted or unsubstituted dibenzofuran group.

In an embodiment, in Formula 1, at least one of R₁ to R₉ and Ar₁ may be a deuterium atom or a substituent including a deuterium atom.

In an embodiment, the amine compound represented by Formula 1 may be a monoamine compound.

In an embodiment, the amine compound may be selected from Compound Group 1, which is explained below.

Another embodiment provides a light-emitting element which may include a first electrode, a second electrode disposed on the first electrode, and at least one functional layer disposed between the first electrode and the second electrode and including an amine compound according to embodiments.

In an embodiment, the at least one functional layer may include a light-emitting layer, a hole transport region disposed between the first electrode and the light-emitting layer, and an electron transport region disposed between the light-emitting layer and the second electrode; and the hole transport region may include the amine compound.

In an embodiment, the hole transport region may include at least one of a hole injection layer, a hole transport layer, or an electron blocking layer; and the at least one of the hole injection layer, the hole transport layer, or the electron blocking layer may include the amine compound.

In an embodiment, the hole transport region may include a first hole transport layer and a second hole transport layer which are sequentially stacked between the first electrode and the light-emitting layer; the first hole transport layer and the second hole transport layer may include different hole transport materials; and the second hole transport layer may include the amine compound.

In an embodiment, the light-emitting layer may include a compound represented by Formula E-1, which is explained below.

In an embodiment, the light-emitting element may further include a capping layer disposed on the second electrode, wherein the capping layer may have a refractive index equal to or greater than about 1.6.

### BRIEF DESCRIPTION OF THE DRAWINGS

The accompanying drawings are included to provide a further understanding of the embodiments, and are incorporated in and constitute a part of this specification. The drawings illustrate embodiments of the disclosure and principles thereof. The above and other aspects and features of the disclosure will become more apparent by describing in detail embodiments thereof with reference to the accompanying drawings, in which:

FIG. 1 is a plan view illustrating a display device according to an embodiment;

FIG. 2 is a schematic cross-sectional view of a display device according to an embodiment;

FIG. 3 is a schematic cross-sectional view illustrating a light-emitting element according to an embodiment;

FIG. 4 is a schematic cross-sectional view illustrating a light-emitting element according to an embodiment;

FIG. 5 is a schematic cross-sectional view illustrating a light-emitting element according to an embodiment;

FIG. 6 is a schematic cross-sectional view illustrating a light-emitting element according to an embodiment;

FIG. 7 is a schematic cross-sectional view illustrating a light-emitting element according to an embodiment;

FIG. 8 is a schematic cross-sectional view illustrating a display device according to an embodiment;

FIG. 9 is a schematic cross-sectional view illustrating a display device according to an embodiment;

FIG. 10 is a schematic cross-sectional view illustrating a display device according to an embodiment; and

FIG. 11 is a schematic cross-sectional view illustrating a display device according to an embodiment.

### DETAILED DESCRIPTION OF THE EMBODIMENTS

The disclosure will now be described more fully hereinafter with reference to the accompanying drawings, in which embodiments are shown. This disclosure may, however, be embodied in different forms and should not be construed as limited to the embodiments set forth herein. Rather, these embodiments are provided so that this disclosure will be thorough and complete, and will fully convey the scope of the disclosure to those skilled in the art.

In the drawings, the sizes, thicknesses, ratios, and dimensions of the elements may be exaggerated for ease of description and for clarity. Like numbers refer to like elements throughout.

In the specification, it will be understood that when an element (or region, layer, part, etc.) is referred to as being “on”, “connected to”, or “coupled to” another element, it can be directly on, connected to, or coupled to the other element, or one or more intervening elements may be present therebetween. In a similar sense, when an element (or region, layer, part, etc.) is described as “covering” another element, it can directly cover the other element, or one or more intervening elements may be present therebetween.

In the specification, when an element is “directly on,” “directly connected to,” or “directly coupled to” another element, there are no intervening elements present. For example, “directly on” may mean that two layers or two elements are disposed without an additional element such as an adhesion element therebetween.

As used herein, the expressions used in the singular such as “a,” “an,” and “the,” are intended to include the plural forms as well, unless the context clearly indicates otherwise.

As used herein, the term “and/or” includes any and all combinations of one or more of the associated listed items. For example, “A and/or B” may be understood to mean “A, B, or A and B.” The terms “and” and “or” may be used in the conjunctive or disjunctive sense and may be understood to be equivalent to “and/or”.

The term “at least one of” is intended to include the meaning of “at least one selected from the group of” for the purpose of its meaning and interpretation. For example, “at least one of A and B” may be understood to mean “A, B, or A and B.” When preceding a list of elements, the term, “at least one of,” modifies the entire list of elements and does not modify the individual elements of the list.

It will be understood that, although the terms first, second, etc. may be used herein to describe various elements, these elements should not be limited by these terms. These terms are only used to distinguish one element from another element. Thus, a first element could be termed a second element without departing from the teachings of the disclosure. Similarly, a second element could be termed a first element, without departing from the scope of the disclosure.

The spatially relative terms “below”, “beneath”, “lower”, “above”, “upper”, or the like, may be used herein for ease of description to describe the relations between one element or component and another element or component as illustrated in the drawings. It will be understood that the spatially relative terms are intended to encompass different orientations of the device in use or operation, in addition to the orientation depicted in the drawings. For example, in the case where a device illustrated in the drawing is turned over, the device positioned “below” or “beneath” another device may be placed “above” another device. Accordingly, the illustrative term “below” may include both the lower and upper positions. The device may also be oriented in other directions and thus the spatially relative terms may be interpreted differently depending on the orientations.

The terms “about” or “approximately” as used herein is inclusive of the stated value and means within an acceptable range of deviation for the recited value as determined by one of ordinary skill in the art, considering the measurement in question and the error associated with measurement of the recited quantity (i.e., the limitations of the measurement system). For example, “about” may mean within one or more standard deviations, or within ±20%, ±10%, or ±5% of the stated value.

It should be understood that the terms “comprises,” “comprising,” “includes,” “including,” “have,” “having,” “contains,” “containing,” and the like are intended to specify the presence of stated features, integers, steps, operations, elements, components, or combinations thereof in the disclosure, but do not preclude the presence or addition of one or more other features, integers, steps, operations, elements, components, or combinations thereof.

Unless otherwise defined or implied herein, all terms (including technical and scientific terms) used have the same meaning as commonly understood by those skilled in the art to which this disclosure pertains. It will be further understood that terms, such as those defined in commonly used dictionaries, should be interpreted as having a meaning that is consistent with their meaning in the context of the relevant art and should not be interpreted in an ideal or excessively formal sense unless clearly defined in the specification.

In the specification, the term “substituted or unsubstituted” may mean a group that is substituted or unsubstituted with at least one substituent selected from the group consisting of a deuterium atom, a halogen atom, a cyano group, a nitro group, an amino group, a silyl group, a oxy group, a thio group, a sulfinyl group, a sulfonyl group, a carbonyl group, a boron group, a phosphine oxide group, a phosphine sulfide group, an alkyl group, an alkenyl group, an alkynyl group, an alkoxy group, a hydrocarbon ring group, an aryl group, and a heterocyclic group. Each of the substituents listed above may itself be substituted or unsubstituted. For example, a biphenyl group may be interpreted as an aryl group, or it may be interpreted as a phenyl group substituted with a phenyl group.

In the specification, the term “bonded to an adjacent group to form a ring” may mean a group that is bonded to an adjacent group to form a substituted or unsubstituted hydrocarbon ring or a substituted or unsubstituted heterocycle. The hydrocarbon ring may be an aliphatic hydrocarbon ring or an aromatic hydrocarbon ring. The heterocycle may be an aliphatic heterocycle or an aromatic heterocycle. The hydrocarbon ring and the heterocycle may each independently be monocyclic or polycyclic. A ring that is formed by the bonding of adjacent groups may itself be connected to another ring to form a spiro structure.

In the specification, the term “adjacent group” may mean a substituent substituted at an atom directly linked to an atom substituted with the corresponding substituent, another substituent substituted at an atom substituted with the corresponding substituent, or a substituent which is three-dimensionally closest to the corresponding substituent. For example, in 1,2-dimethylbenzene, two methyl groups may be interpreted as “adjacent groups” to each other, and in 1,1-diethylcyclopentane, two ethyl groups may be interpreted as “adjacent groups” to each other. For example, in 4,5-dimethylphenanthrene, two methyl groups may be interpreted as “adjacent groups” to each other.

In the specification, examples of a halogen atom may include a fluorine atom, a chlorine atom, a bromine atom, or an iodine atom.

In the specification, an alkyl group may be straight, branched, or cyclic. The number of carbon atoms in the alkyl group may be 1 to 50, 1 to 30, 1 to 20, 1 to 10, or 1 to 6. Examples of the alkyl group may include a methyl group, an ethyl group, an n-propyl group, a isopropyl group, an n-butyl group, a s-butyl group, a t-butyl group, an i-butyl group, an 2-ethylbutyl group, a 3, 3-dimethylbutyl group, an n-pentyl group, an i-pentyl group, a neopentyl group, a t-pentyl group, a cyclopentyl group, a 1-methylpentyl group, a 3-methylpentyl group, an 2-ethylpentyl group, a 4-methyl-2-pentyl group, an n-hexyl group, a 1-methylhexyl group, an 2-ethylhexyl group, a 2-butylhexyl group, a cyclohexyl group, a 4-methylcyclohexyl group, a 4-t-butylcyclohexyl group, an n-heptyl group, a 1-methylheptyl group, a 2,2-dimethylheptyl group, an 2-ethylheptyl group, a 2-butylheptyl group, an n-octyl group, a t-octyl group, an 2-ethyloctyl group, a 2-butyloctyl group, an 2-hexyloctyl group, a 3,7-dimethyloctyl group, a cyclooctyl group, an n-nonyl group, an n-decyl group, an adamantyl group, an 2-ethyldecyl group, a 2-butyldecyl group, a 2-hexyldecyl group, an 2-octyldecyl group, an n-undecyl group, an n-dodecyl group, an 2-ethyldodecyl group, a 2-butyldodecyl group, an 2-hexyldodecyl group, an 2-octyldodecyl group, an n-tridecyl group, an n-tetradecyl group, an n-pentadecyl group, an n-hexadecyl group, an 2-ethylhexadecyl group, a 2-butylhexadecyl group, an 2-hexylhexadecyl group, an 2-octylhexadecyl group, an n-heptadecyl group, an n-octadecyl group, an n-nonadecyl group, an n-icosyl group, an 2-ethylicosyl group, a 2-butylicosyl group, an 2-hexylicosyl group, an 2-octylicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, and the like, but embodiments are not limited thereto.

In the specification, an alkenyl group may be a hydrocarbon group including one or more carbon-carbon double bonds in the middle or at a terminus of an alkyl group having 2 or more carbon atoms. The alkenyl group may be straight or branched. The number of carbon atoms in the alkenyl group is not particularly limited, but may be 2 to 30, 2 to 20, or 2 to 10. Examples of the alkenyl group may include a vinyl group, a 1-butenyl group, a 1-pentenyl group, a 1,3-butadienyl group, a styrenyl group, a styryl vinyl group, and the like, but embodiments are not limited thereto.

In the specification, a hydrocarbon ring group may be any functional group or substituent derived from an aliphatic hydrocarbon ring or an aromatic hydrocarbon ring. For example, the hydrocarbon ring group may be a saturated hydrocarbon ring group having 5 to 20 ring carbon atoms.

In the specification, an aryl group may be any functional group or substituent derived from an aromatic hydrocarbon ring. The aryl group may be a monocyclic aryl group or a polycyclic aryl group. The number of ring-forming carbon atoms in the aryl group may be 6 to 30, 6 to 20, or 6 to 15. Examples of the aryl group may include a phenyl group, a naphthyl group, a fluorenyl group, an anthracenyl group, a phenanthryl group, a biphenyl group, a terphenyl group, a quarterphenyl group, a quinquephenyl group, a sexyphenyl group, a triphenylenyl group, a pyrenyl group, a benzofluoranthenyl group, a chrysenyl group, and the like, but embodiments are not limited thereto.

In the specification, a fluorenyl group may be substituted, and two substituents may be bonded to each other to form a spiro structure. Examples of substituted fluorenyl groups are as follows. However, embodiments are not limited thereto.

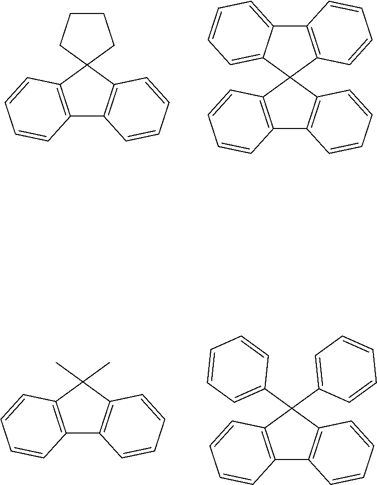

In the specification, a heterocyclic group may be any functional group or substituent derived from a ring containing at least one of B, O, N, P, Si, or S as a heteroatom. The heterocyclic group may be an aliphatic heterocyclic group or an aromatic heterocyclic group. The aromatic heterocyclic group may be a heteroaryl group. The aliphatic heterocycle and the aromatic heterocycle may each independently be monocyclic or polycyclic.

In the specification, a heterocyclic group may include at least one of B, O, N, P, Si, and S as a heteroatom. When the heterocyclic group includes two or more heteroatoms, the two or more heteroatoms may be the same as or different from each other. In the specification, the heterocyclic group may be monocyclic group or a polycyclic group, and the heterocyclic group may be a heteroaryl group. The number of ring-forming carbon atoms in the heterocyclic group may be 2 to 30, 2 to 20, or 2 to 10.

In the specification, an aliphatic heterocyclic group may include at least one of B, O, N, P, Si, and S as a heteroatom. The number of ring-forming carbon atoms in the aliphatic heterocyclic group may be 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, etc., but embodiments are not limited thereto.

In the specification, a heteroaryl group may include, as a heteroatom, at least one of B, O, N, P, Si, or S. When the heteroaryl group includes two or more heteroatoms, the two or more heteroatoms may be the same as or different from each other. The heteroaryl group may be a monocyclic group or a polycyclic group. The number of ring-forming carbon atoms in the heterocyclic group may be 2 to 30, 2 to 20, or 2 to 10. Examples of the heteroaryl group may include a thiophene group, a furan group, a pyrrole group, a imidazole group, a triazole group, a pyridine group, a bipyridine group, a pyrimidine group, a triazine group, a triazole group, a triazole group, an acridyl group, a pyridazine group, a pyrazinyl group, a quinoline group, a quinazoline group, a quinoxaline group, a phenoxy group, a phthalazine group, a pyrido pyrimidine group, a pyrido pyrazine group, a pyrazino pyrazine group, an isoquinoline group, an indole group, a carbazole group, an N-aryl carbazole group, an N-heteroaryl carbazole group, an N-alkylcarbazole group, a benzoxazole group, a benzoimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, and the like, but embodiments are not limited thereto.

In the specification, the description of the above-described aryl group may be applied to an arylene group, except that the arylene group is a divalent group. The description of the above-described heteroaryl group may be applied to a heteroarylene group, except that the heteroarylene group is a divalent group.

In the specification, a boryl group may be an alkyl boryl group or an aryl boryl group. Examples of the boryl group may include a trimethylboryl group, a triethylboryl group, a t-butyldimethylboryl group, a triphenylboryl group, a diphenylboryl group, a phenylboryl group, and the like, but embodiments are not limited thereto. For example, the alkyl group in the alkylboryl group may be the same as the alkyl group as described above, and the aryl group in the arylboryl group may be the same as the aryl group as described above.

In the specification, a silyl group may be an alkyl silyl group or an aryl silyl group. Examples of the silyl group may include a trimethylsilyl group, a triethylsilyl group, a t-butyldimethylsilyl group, a vinyl dimethylsilyl group, a propyldimethylsilyl group, a triphenylsilyl group, a diphenylsilyl group, a phenylsilyl group, and the like, but embodiments are not limited thereto.

In the specification, the number of carbon atoms in a carbonyl group is not particularly limited, but may be 1 to 40, 1 to 30, or 1 to 20. For example, the carbonyl group may have one of the structures as shown below, but is not limited thereto.

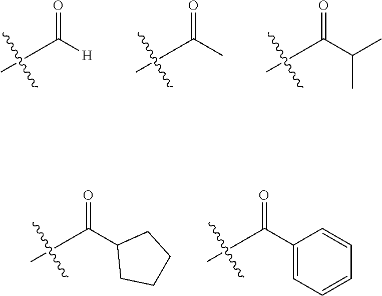

In the specification, the number of carbon atoms in a sulfinyl group and the number of carbon atoms in a sulfonyl group are not particularly limited, but may each independently be 1 to 30. The sulfinyl group may be an alkyl sulfinyl group or an aryl sulfinyl group. The sulfonyl group may be an alkyl sulfonyl group or an aryl sulfonyl group.

In the specification, a thio group may be an alkyl thio group or an aryl thio group. The thio group may be a sulfur atom that is bonded to an alkyl group or an aryl group as defined above. Examples of the thio group may include a methylthio group, an ethylthio group, a propylthio group, a pentylthio group, an hexylthio group, an octylthio group, a dodecylthio group, a cyclopentylthio group, a cyclohexylthio group, a phenylthio group, a naphthylthio group, and the like, but embodiments are not limited thereto.

In the specification, an oxy group may be an oxygen atom that is bonded to an alkyl group or an aryl group as defined above. The oxy group may be an alkoxy group or an aryl oxy group. The alkoxy group may be linear, branched, or cyclic. The number of carbon atoms in the alkoxy group is not particularly limited but may be, for example, 1 to 20 or 1 to 10. Examples of the oxy group may include methoxy, ethoxy, n-propoxy, isopropoxy, butoxy, pentyloxy, hexyloxy, octyloxy, nonyloxy, decyloxy, benzyloxy, and the like. However, embodiments are not limited thereto.

In the specification, the number of carbon atoms in an amine group is not particularly limited, but may be 1 to 30. The amine group may be an alkyl amine group or an aryl amine group. Examples of the amine group may include a methylamine group, a dimethylamine group, a phenylamine group, a diphenylamine group, a naphthylamine group, a 9-methyl-anthracenylamine group, and a triphenylamine group, but embodiments are not limited thereto.

In the specification, an alkyl group in an alkyl thio group, an alkyl sulfonyl group, an alkyl aryl group, an alkyl amino group, an alkyl boron group, an alkyl silyl group, or an alkyl amine group, may be the same as the alkyl group as defined above.

In the specification, an aryl group in an aryl oxy group, an aryl thio group, an aryl sulfonyl group, an aryl amino group, an aryl boron group, an aryl silyl group, or an aryl amine group, may be the same as the aryl group as defined above.

In the specification, a direct linkage may be a single bond.

In the specification, the symbols

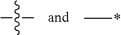

each represents a bonding site to a neighboring atom.

Hereinafter, a light-emitting element and an amine compound according to embodiments will be described with reference to the drawings.

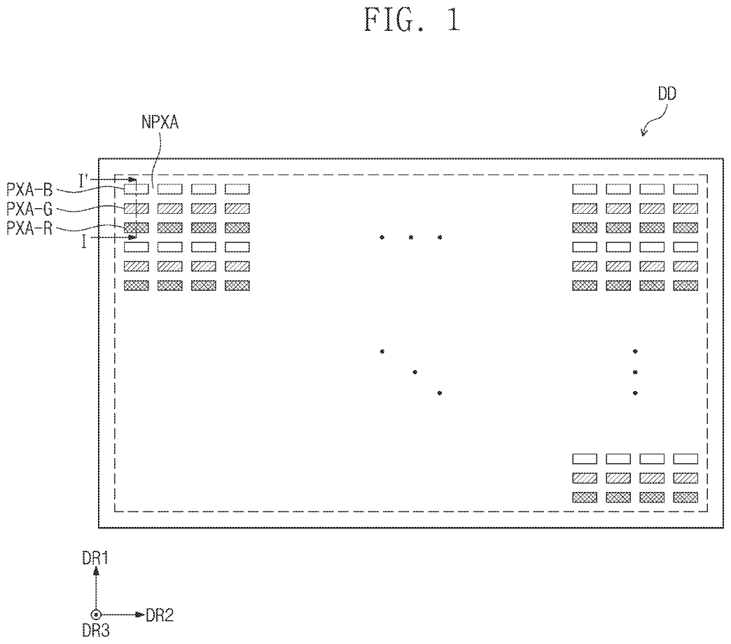

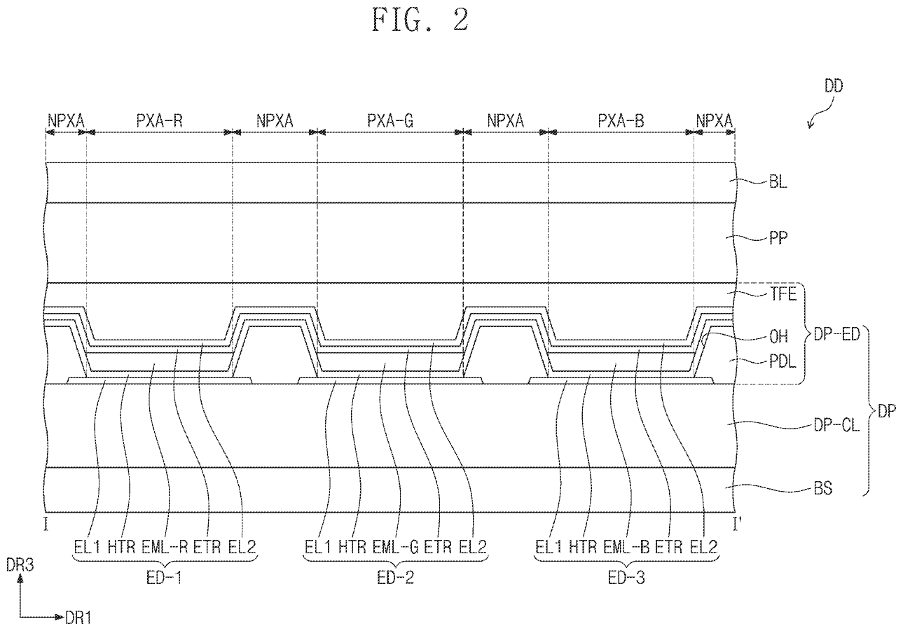

FIG. 1 is a plan view illustrating an embodiment of a display device DD. FIG. 2 is a schematic cross-sectional view of a display device DD according to an embodiment. FIG. 2 is a schematic cross-sectional view taken along line I-I′ of FIG. 1.

The display device DD may include a display panel DP and an optical layer PP disposed on the display panel DP. The display panel DP may include light-emitting elements ED-1, ED-2, and ED-3. The display device DD may include multiples of each of the light-emitting elements ED-1, ED-2, and ED-3. The optical layer PP may be disposed on the display panel DP and may control light reflected at the display panel DP from an external light. For example, the optical layer PP may include a polarizing layer or a color filter layer. Although not shown in the drawings, in an embodiment, the optical layer PP may be omitted from the display device DD.

A base substrate BL may be disposed on the optical layer PP. The base substrate BL may provide a base surface on which the optical layer PP is disposed. The base substrate BL may be a glass substrate, a metal substrate, a plastic substrate, or the like. However, embodiments are not limited thereto, and the base substrate BL may include an inorganic layer, an organic layer, or a composite material layer. Although not shown in the drawings, in an embodiment, the base substrate BL may be omitted.

The display device DD according to an embodiment may further include a filling layer (not shown). The filling layer (not shown) may be disposed between the display element layer DP-ED and the base substrate BL. The filling layer (not shown) may be an organic material layer. The filling layer (not shown) may include at least one of an acrylic resin, a silicone-based resin, or an epoxy-based resin.

The display panel DP may include a base layer BS, a circuit layer DP-CL provided on the base layer BS, and a display element layer DP-ED. The display element layer DP-ED includes pixel defining layer PDL, light-emitting elements ED-1, ED-2, and ED-3 disposed between portions of the pixel defining layer PDL, and an encapsulation layer TFE disposed on the light-emitting elements ED-1, ED-2, and ED-3.

The base layer BS may provide a base surface on which the display element layer DP-ED is disposed. The base layer BS may be a glass substrate, a metal substrate, a plastic substrate, and the like. However, embodiments are not limited thereto, and the base layer BS may include an inorganic layer, an organic layer, or a composite material layer.

In an embodiment, the circuit layer DP-CL may be disposed on the base layer BS, and may include transistors (not shown). The transistors (not shown) may each include a control electrode, an input electrode, and an output electrode. For example, the circuit layer DP-CL may include a switching transistor and a driving transistor for driving the light-emitting elements ED-1, ED-2, and ED-3 of the display element layer DP-ED.

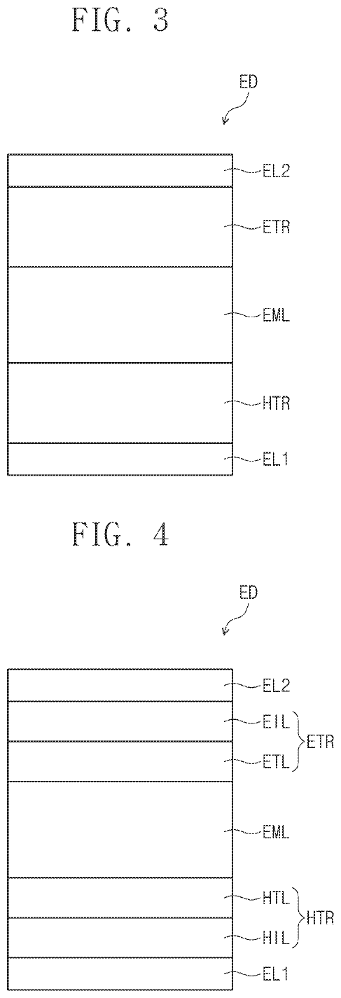

Each of the light-emitting elements ED-1, ED-2, and ED-3 may have a structure of a light-emitting element ED according to an embodiment in FIGS. 3 to 7 to be described later. Each of the light-emitting elements ED-1, ED-2, and ED-3 may include a first electrode EL1, a hole transport region HTR, light-emitting layers EML-R, EML-G, and EML-B, an electron transport region ETR, and a second electrode EL2.

FIG. 2 illustrates an embodiment in which the light-emitting layers EML-R, EML-G, and EML-B of the light-emitting elements ED-1, ED-2, and ED-3 are disposed in openings OH defined in the pixel defining layer PDL, and the hole transport region HTR, the electron transport region ETR, and the second electrode EL2 are each provided as common layers for all of the light-emitting elements ED-1, ED-2, ED-3. However, embodiments are not limited thereto. Although not shown in FIG. 2, in an embodiment, the hole transport region HTR and the electron transport region ETR may each be provided by being patterned inside the openings OH defined in the pixel defining layer PDL. For example, in an embodiment, the hole transport region HTR, the light-emitting layers EML-R, EML-G, and EML-B, and the electron transport region ETR of the light-emitting elements ED-1, ED-2, and ED-3 may each be provided by being patterned through an inkjet printing method.

The encapsulation layer TFE may cover the light-emitting elements ED-1, ED-2, and ED-3. The encapsulation layer TFE may seal the display element layer DP-ED. The encapsulation layer TFE may be a thin film encapsulation layer. The encapsulation layer TFE may be a single layer or multiple layers. The encapsulation layer TFE may include at least one insulating layer. The encapsulation layer TFE according to an embodiment may include at least one inorganic film (hereinafter, an inorganic encapsulation film). In an embodiment, the encapsulation layer TFE may include at least one organic film (hereinafter, an organic encapsulation film) and at least one inorganic encapsulation film.

The inorganic encapsulation film may protect the display element layer DP-ED from moisture and/or oxygen, and the organic encapsulation film may protect the display device layer DP-ED from foreign substances such as dust particles. The inorganic encapsulation film may include silicon nitride, silicon oxynitride, silicon oxide, titanium oxide, aluminum oxide, or the like, but embodiments are not limited thereto. The organic encapsulation film may include an acrylic compound, an epoxy-based compound, or the like. The organic encapsulation film may include a photopolymerizable organic material, but embodiments are not limited thereto.

The encapsulation layer TFE may be disposed on the second electrode EL2, and may be disposed to fill the openings OH.

Referring to FIGS. 1 and 2, the display device DD may include a non-light-emitting region NPXA and light-emitting regions PXA-R, PXA-G, and PXA-B. The light-emitting regions PXA-R, PXA-G, and PXA-B may each be a region in which light generated by the respective light-emitting elements ED-1, ED-2, and ED-3 is emitted. The light-emitting regions PXA-R, PXA-G, and PXA-B may be spaced apart from each other in a plan view.

The light-emitting regions PXA-R, PXA-G, and PXA-B may each be a region separated by the pixel defining layer PDL. The non-light-emitting regions NPXA may be regions between the adjacent light-emitting regions PXA-B, PXA-G, and PXA-R and may correspond to the pixel defining layer PDL. For example, an in embodiment, the light-emitting regions PXA-B, PXA-G, and PXA-R may each correspond to a pixel. The pixel defining layer PDL may separate the light-emitting elements ED-1, ED-2, and ED-3. The light-emitting layers EML-R, EML-G, EML-B of the light-emitting elements ED-1, ED-2, and ED-3 may be disposed in the openings OH defined in the pixel defining layer PDL and thus separated from each other.

The light-emitting regions PXA-R, PXA-G, and PXA-G may be arranged into groups according to the color of light generated from the light-emitting elements ED-1, ED-2, and ED-3. In the display device DD according to an embodiment illustrated in FIGS. 1 and 2, three light-emitting regions PXA-R, PXA-G, and PXA-B respectively emitting red light, green light, and blue light are illustrated as an example. For example, the display device DD according to an embodiment may include a red light-emitting region PXA-R, a green light-emitting region PXA-G, and a blue light-emitting region PXA-B that are distinguished from each other.

In the display device DD according to an embodiment, the light-emitting elements ED-1, ED-2, and ED-3 may emit light having different wavelength ranges from each other. For example, in an embodiment, the display device DD may include a first light-emitting element ED-1 that emits red light, a second light-emitting element ED-2 that emits green light, and a third light-emitting element ED-3 that emits blue light. For example, the red light-emitting region PXA-R, the green light-emitting region PXA-G, and the blue light-emitting region PXA-B of the display device DD may respectively correspond to the first light-emitting element ED-1, the second light-emitting element ED-2, and the third light-emitting element ED-3.

However, embodiments are not limited thereto, and the first to third light-emitting elements ED-1, ED-2, and ED-3 may emit light in a same wavelength range or at least one of the first to third light-emitting elements ED-1, ED-2, or ED-3 may emit light of a different wavelength range. For example, all of the first to third light-emitting elements ED-1, ED-2, and ED-3 may emit blue light.

The light-emitting regions PXA-R, PXA-G, and PXA-B in the display device DD according to an embodiment may be arranged in a stripe configuration. Referring to FIG. 1, the red light-emitting regions PXA-R, the green light-emitting regions PXA-G, and the blue light-emitting regions PXA-B may each be aligned along a second direction axis DR2. In another embodiment, the light-emitting regions may be alternately arranged in the order of the red light-emitting region PXA-R, the green light-emitting region PXA-G, and the blue light-emitting region PXA-B along a first direction axis DR1.

FIGS. 1 and 2 illustrate that areas of the light-emitting regions PXA-R, PXA-G, and PXA-B are all similar, but embodiments are not limited thereto. Thus, the areas of the light-emitting regions PXA-R, PXA-G, and PXA-B may be different from each other according to a wavelength range of the emitted light. The areas of the light-emitting regions PXA-R, PXA-G, and PXA-B may be areas in a plan view that are defined by the first direction axis DR1 and the second direction axis DR2.

An arrangement form of the light-emitting regions PXA-R, PXA-G, and PXA-B is not limited to what is illustrated in FIG. 1, and the order in which the red light-emitting region PXA-R, the green light-emitting region PXA-G, and the blue light-emitting region PXA-B are arranged may be provided in various combinations according to the display quality characteristics which are required for the display device DD. For example, the arrangement form of the light-emitting regions PXA-R, PXA-G, and PXA-B may be a PENTILE® configuration or a Diamond Pixel™ configuration.

In an embodiment, the areas of the light-emitting regions PXA-R, PXA-G, and PXA-B may be different in size from each other. For example, in an embodiment, an area of the green light-emitting region PXA-G may be smaller than an area of the blue light-emitting region PXA-B, but embodiments are not limited thereto.

Hereinafter, FIGS. 3 to 7 are schematic cross-sectional views illustrating light-emitting elements according to an embodiments. Each of the light-emitting elements ED according to embodiments may include a first electrode, a second electrode EL2 facing the first electrode EL1, and at least one functional layer disposed between the first electrode EL1 and the second electrode EL2. The light-emitting element ED according to an embodiment may include an amine compound according to an embodiment to be described later in at least one functional layer.

The light-emitting element ED may include, as the at least one functional layer, a hole transport region HTR, a light-emitting layer EML, an electron transport region ETR, etc., which are sequentially stacked. Referring to FIG. 3, the light-emitting element ED according to an embodiment may include a first electrode EL1, a hole transport region HTR, a light-emitting layer EML, an electron transport region ETR, and a second electrode EL2 that are stacked in that order.

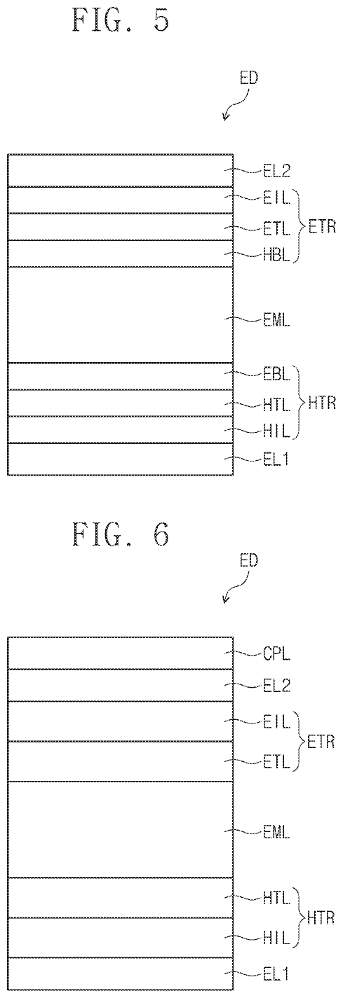

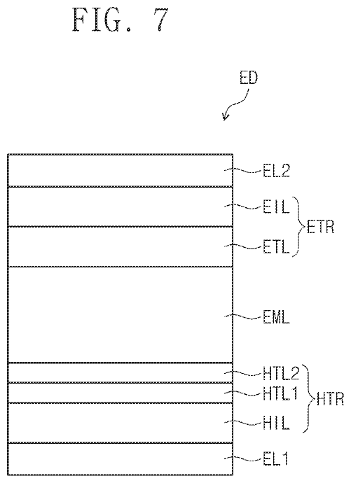

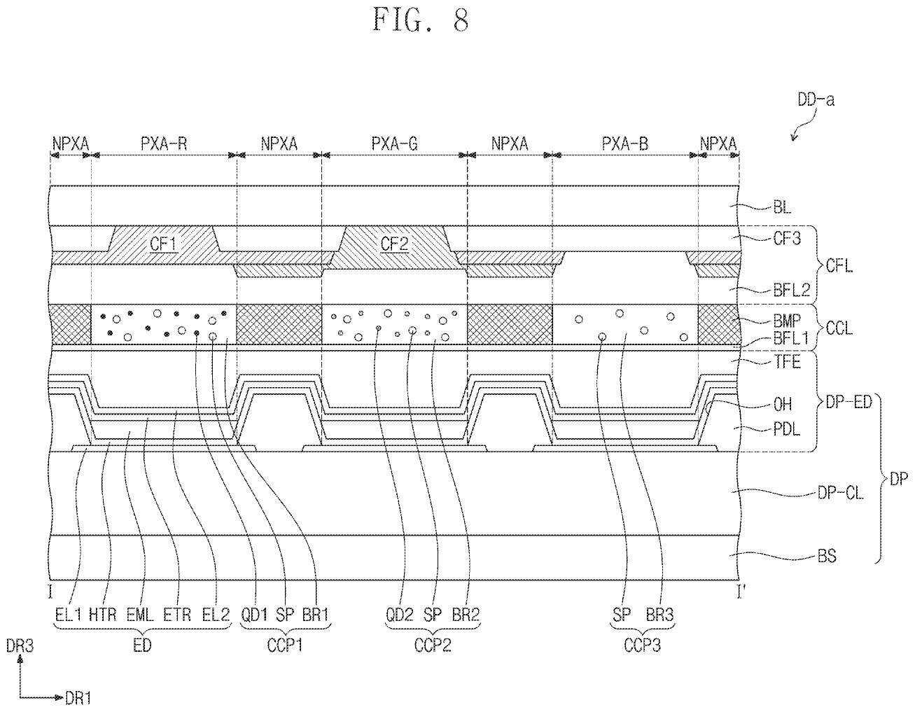

FIG. 4 is a schematic cross-sectional view of a light-emitting element ED according to an embodiment in which the hole transport region HTR includes a hole injection layer HIL and a hole transport layer HTL, and the electron transport region ETR includes an electron injection layer EIL and an electron transport layer ETL, as compared with FIG. 3. FIG. 5 is a schematic cross-sectional view of a light-emitting element ED according to an embodiment in which the hole transport region HTR includes a hole injection layer HIL, a hole transport layer HTL, and an electron blocking layer EBL, and the electron transport region ETR includes an electron injection layer EIL, an electron transport layer ETL, and a hole blocking layer HBL, as compared with FIG. 3. FIG. 6 is a schematic cross-sectional view of a light-emitting element ED according to an embodiment including a capping layer CPL disposed on the second electrode EL2, as compared with FIG. 4. FIG. 7 is a schematic cross-sectional view of a light-emitting element ED according to an embodiment in which the hole transport region HTR includes hole transport layers HTL1 and HTL2, as compared with FIG. 4.

The light-emitting element ED according to an embodiment may include, in the hole transport region HTR, an amine compound according to an embodiment to be described later. In the light-emitting element ED according to an embodiment, at least one of the hole injection layer HIL, the hole transport layer HTL, or the electron blocking layer EBL in the hole transport region HTR may include the amine compound according to an embodiment, or at least one of the first hole transport layer HTL1 or the second hole transport layer HTL2 may include the amine compound according to an embodiment.

In the light-emitting element ED according to an embodiment, the first electrode EL1 has conductivity. The first electrode EL1 may be formed of a metal material, a metal alloy, or a conductive compound. The first electrode EL1 may be an anode or a cathode. However, embodiments are not limited thereto. For example, the first electrode EL1 may be a pixel electrode. The first electrode EL1 may be a transmissive electrode, a transflective electrode, or a reflective electrode. The first electrode EL1 may include at least one of Ag, Mg, Cu, Al, Pt, Pd, Au, Ni, Nd, Ir, Cr, Li, Ca, LiF, Mo, Ti, W, In, Sn, Zn, an oxide thereof, a compound thereof, or a mixture thereof.

When the first electrode EL1 is a transmissive electrode, the first electrode EL1 may include a transparent metal oxide such as indium tin oxide (ITO), indium zinc oxide (IZO), zinc oxide (ZnO), or indium tin zinc oxide (ITZO). When the first electrode EL1 is a transflective electrode or a reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, compounds thereof, or mixtures thereof (for example, a mixture of Ag and Mg). In another embodiment, the first electrode EL1 may have a multilayer structure including a reflective film or a transflective film formed of the above materials, and a transmissive conductive film formed of ITO, IZO, ZnO, ITZO, or the like. For example, the first electrode EL1 may include a three-layer structure of ITO/Ag/ITO. However, embodiments are not limited thereto. The first electrode EL1 may include the above-described metal materials, combinations of two or more metal materials selected from among the above-described metal materials, or oxides of the above-described metal materials. The first electrode EL1 may have a thickness in a range of about 700 Å to about 10,000 Å. For example, the first electrode EL1 may have a thickness in a range of about 1,000 Å to about 3,000 Å.

The hole transport region HTR is provided on the first electrode EL1. The hole transport region HTR may be a layer formed of a single material, a layer formed of different materials, or a structure having multiple layers formed of different materials.

The hole transport region HTR may include at least one of a hole injection layer HIL, a hole transport layer HTL, a buffer layer (not shown), or an electron blocking layer EBL. In an embodiment, the hole transport region HTR may have a structure of a hole injection layer HIL, a first hole transport layer HTL1, and a second hole transport layer HTL2, stacked in that order.

For example, the hole transport region HTR may have a structure of a single layer of a hole injection layer HIL or a hole transport layer HTL, and may have a structure of a single layer formed of a hole injection material and a hole transport material. In an embodiment, the hole transport region HTR may have a structure of a single layer formed of different materials, or may have a structure in which a hole injection layer HIL/hole transport layer HTL, a hole injection layer HIL/hole transport layer HTL/buffer layer (not shown), a hole injection layer HIL/buffer layer (not shown), or a hole transport layer HTL/buffer layer (not shown), are stacked in its respective stated order from the first electrode EL1, but embodiments are not limited thereto.

The hole transport region HTR may have a thickness, for example, in a range of about 50 Å to about 15,000 Å. The hole transport region HTR may be formed using various methods such as a vacuum deposition method, a spin coating method, a cast method, a Langmuir-Blodgett (LB) method, an inkjet printing method, a laser printing method, and a laser induced thermal imaging (LITI) method.

The light-emitting element ED according to an embodiment may include, in the hole transport region HTR, an amine compound according to an embodiment represented by Formula 1. In the light-emitting element ED according to an embodiment, the hole transport layer HTL may include the amine compound according to an embodiment represented by Formula 1, and in the light-emitting element ED according to an embodiment, the second hole transport layer HTL2 may include the amine compound according to an embodiment represented by Formula 1.

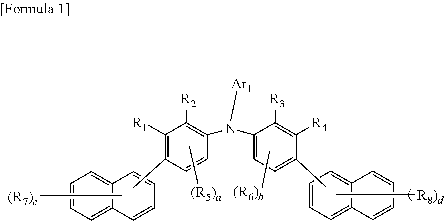

In Formula 1, a pair of R₁ and R₂ and/or a pair of R₃ and R₄ may be bonded with a group represented by Formula 2 to form a ring.

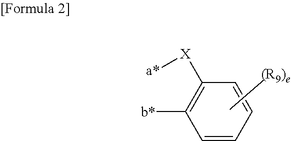

The amine compound according to an embodiment may have at least two naphthyl groups and may include a dibenzoheterole group as a linker moiety between at least one naphthyl group and a nitrogen atom of an amine moiety. The two naphthyl groups of the amine compound according to an embodiment may be respectively bonded to an aryl group or to a dibenzoheterole moiety at a para position with respect to the nitrogen atom of the amine moiety.

In Formula 1, Ar₁ may be a substituted or unsubstituted aryl group having 6 to 40 ring-forming carbon atoms, or a substituted or unsubstituted heteroaryl group having 5 to 40 ring-forming carbon atoms. In an embodiment, a phenanthryl group having high planarity may be excluded from Ar₁. In an embodiment, Ar₁ may be a substituted or unsubstituted phenyl group, a substituted or unsubstituted naphthyl group, a substituted or unsubstituted dibenzothiophene group, or a substituted or unsubstituted dibenzofuran group. However, embodiments are not limited thereto.

In Formula 1, R₁ to R₄ may each independently be a hydrogen atom, a deuterium atom, a substituted or unsubstituted alkyl group having 1 to 20 carbon atoms, a substituted or unsubstituted aryl group having 6 to 40 ring-forming carbon atoms, or a single bond forming a ring by bonding with a group represented by Formula 2. A pair of R₁ and R₂ and/or a pair of R₃ and R₄ may be bonded with a group represented by Formula 2 to form a ring of a dibenzoheterole moiety.

In Formula 2, X may be O, S, N(R₁₀), or C(R₁₁)(R₁₂), and a* and b* each indicate a bond to one of R₁ to R₄ in Formula 1. For example, a* and b* may be bonded with R₁ and R₂ to form a ring, or a* and b* may be bonded with R₃ and R₄ to form a ring. A ring formed by bonding a pair of R₁ and R₂ and/or a pair of R₃ and R₄ with a group represented by Formula 2 may be a dibenzofuran derivative, a dibenzothiophene derivative, a carbazole derivative, or a fluorene derivative.

In Formula 1 and Formula 2, R₅ to R₁₂ may each independently be a hydrogen atom, a deuterium atom, a substituted or unsubstituted alkyl group having 1 to 20 carbon atoms, or a substituted or unsubstituted aryl group having 6 to 40 ring-forming carbon atoms. In Formula 1 and Formula 2, a and b may each independently be an integer from 0 to 2, c and d may each independently be an integer from 0 to 7, and e may be an integer from 0 to 4.

When a to e are each 2 or more, multiple groups of R₅ to R₉ may be the same as or different from each other. For example, when a is 2, two R₅ groups may be the same as or different from each other. This description may be similarly applied to each of R₆ to R₉.

In an embodiment, in the amine compound represented by Formula 1, at least one of R₁ to R₉ and Ar₁ may be a deuterium atom or a substituent including a deuterium atom. For example, the amine compound according to an embodiment may include at least one deuterium atom as a substituent.

In an embodiment, the amine compound represented by Formula 1 may be a monoamine compound. The amine compound according to an embodiment may not include an amine group as a substituent.

In an embodiment, the amine compound represented by Formula 1 may be represented by any one of Formulas 1-1 to 1-3. In Formulas 1-1 to 1-3, Ar₁, X, R₁ to R₁₂, and a to e may be the same as those described in Formulas 1 and 2.

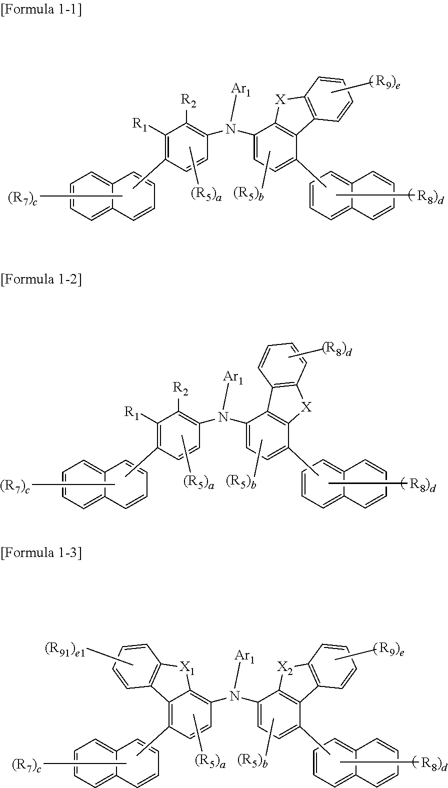

In Formula 1-3, X₁ and X₂ may each independently be O, S, N(R₁₀), or C(R₁₁)(R₁₂); e1 and e2 may each independently be an integer from 0 to 4; and R₉₁ and R₉₂ may each independently be a hydrogen atom, a deuterium atom, a substituted or unsubstituted alkyl group having 1 to 20 carbon atoms, or a substituted or unsubstituted aryl group having 6 to 40 ring-forming carbon atoms.

In Formula 1-3, X₁ and X₂ may be the same as or different from each other. For example, in an embodiment, X₁ and X₂ may be the same, and both X₁ and X₂ may be O or S.

In an embodiment, the group represented by Formula 2 may be represented by any one of Formulas 2-1 to 2-5.

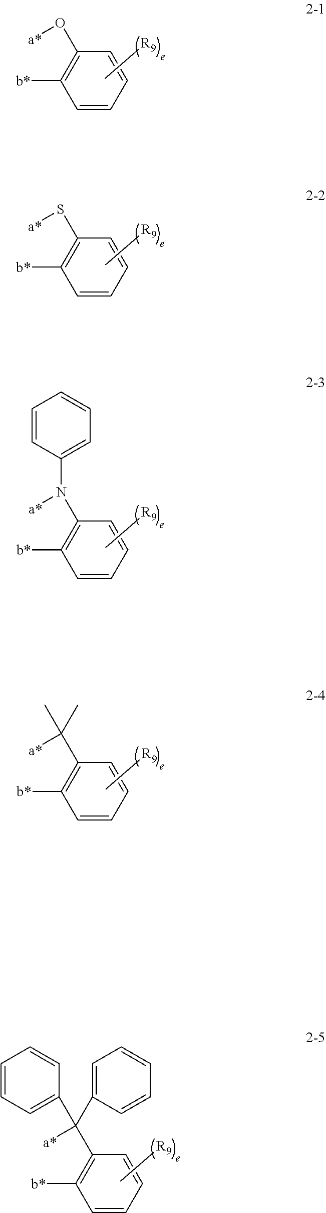

In Formulas 2-1 to 2-5, a* and b* may each indicate a bond to any one of R₁ to R₄ of Formula 1. In Formulas 2-1 to 2-5, R₉, e, a*, and b* are the same as defined in Formula 2.

The amine compound according to an embodiment, represented by Formula 1, may be selected from Compound Group 1. The hole transport region HTR of the light-emitting element ED according to an embodiment may include at least one of the amine compounds in Compound Group 1. In Compound Group 1, D indicates a deuterium atom.

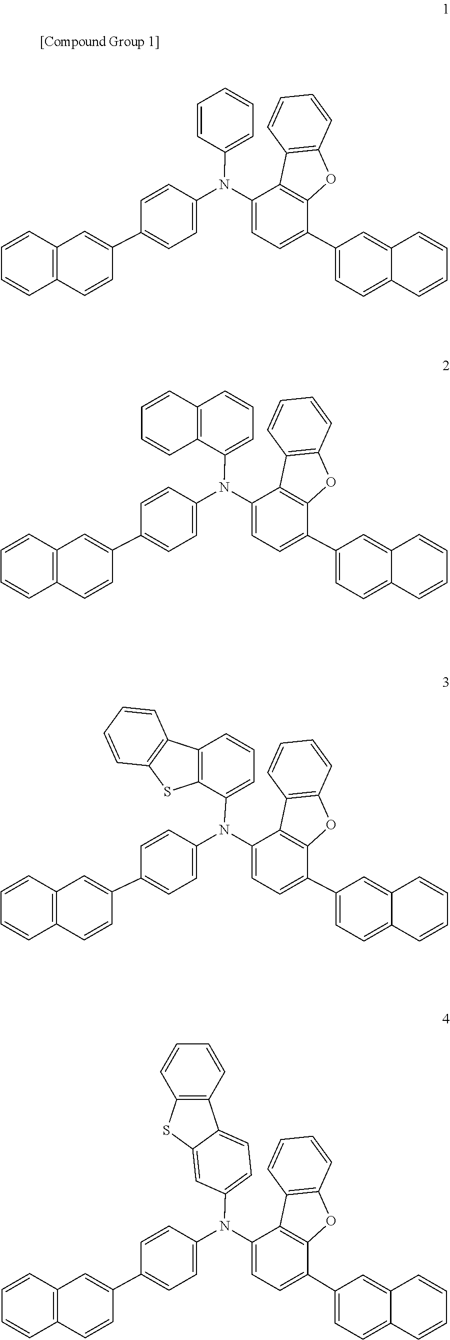 [SMILES: c1ccc(N(c2ccc(-c3ccc4ccccc4c3)cc2)c2ccc(-c3ccc4ccccc4c3)c3oc4ccccc4c23)cc1 ; c1ccc2cc(-c3ccc(N(c4cccc5ccccc45)c4ccc(-c5ccc6ccccc6c5)c5oc6ccccc6c45)cc3)ccc2c1 ; c1ccc2cc(-c3ccc(N(c4cccc5c4sc4ccccc45)c4ccc(-c5ccc6ccccc6c5)c5oc6ccccc6c45)cc3)ccc2c1 ; c1ccc2cc(-c3ccc(N(c4ccc5c(c4)sc4ccccc45)c4ccc(-c5ccc6ccccc6c5)c5oc6ccccc6c45)cc3)ccc2c1]

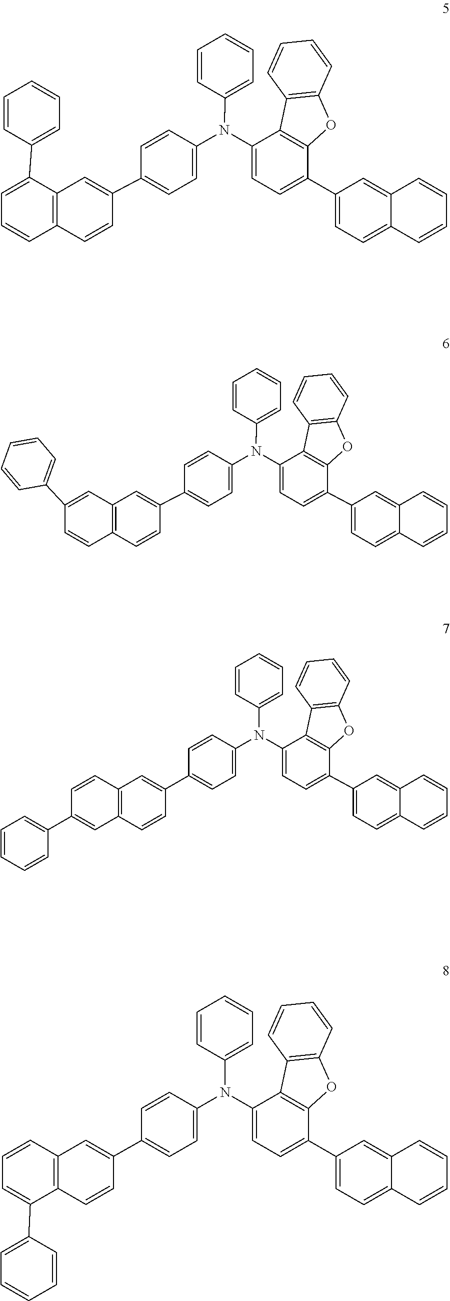 [SMILES: c1ccc(-c2cccc3ccc(-c4ccc(N(c5ccccc5)c5ccc(-c6ccc7ccccc7c6)c6oc7ccccc7c56)cc4)cc23)cc1 ; c1ccc(-c2ccc3ccc(-c4ccc(N(c5ccccc5)c5ccc(-c6ccc7ccccc7c6)c6oc7ccccc7c56)cc4)cc3c2)cc1 ; c1ccc(-c2ccc3cc(-c4ccc(N(c5ccccc5)c5ccc(-c6ccc7ccccc7c6)c6oc7ccccc7c56)cc4)ccc3c2)cc1 ; c1ccc(-c2cccc3cc(-c4ccc(N(c5ccccc5)c5ccc(-c6ccc7ccccc7c6)c6oc7ccccc7c56)cc4)ccc23)cc1]

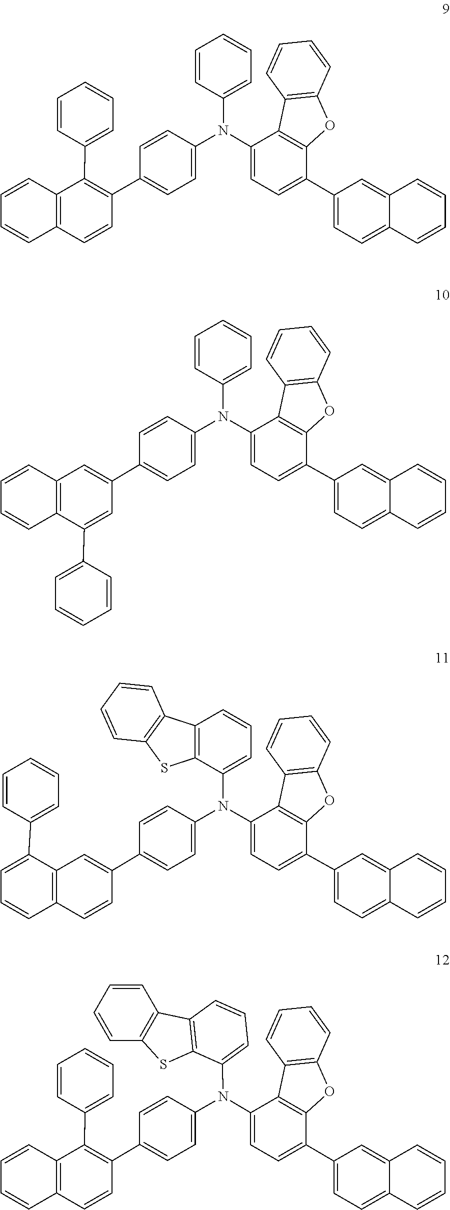 [SMILES: c1ccc(-c2c(-c3ccc(N(c4ccccc4)c4ccc(-c5ccc6ccccc6c5)c5oc6ccccc6c45)cc3)ccc3ccccc23)cc1 ; c1ccc(-c2cc(-c3ccc(N(c4ccccc4)c4ccc(-c5ccc6ccccc6c5)c5oc6ccccc6c45)cc3)cc3ccccc23)cc1 ; c1ccc(-c2cccc3ccc(-c4ccc(N(c5cccc6c5sc5ccccc56)c5ccc(-c6ccc7ccccc7c6)c6oc7ccccc7c56)cc4)cc23)cc1 ; c1ccc(-c2c(-c3ccc(N(c4cccc5c4sc4ccccc45)c4ccc(-c5ccc6ccccc6c5)c5oc6ccccc6c45)cc3)ccc3ccccc23)cc1]

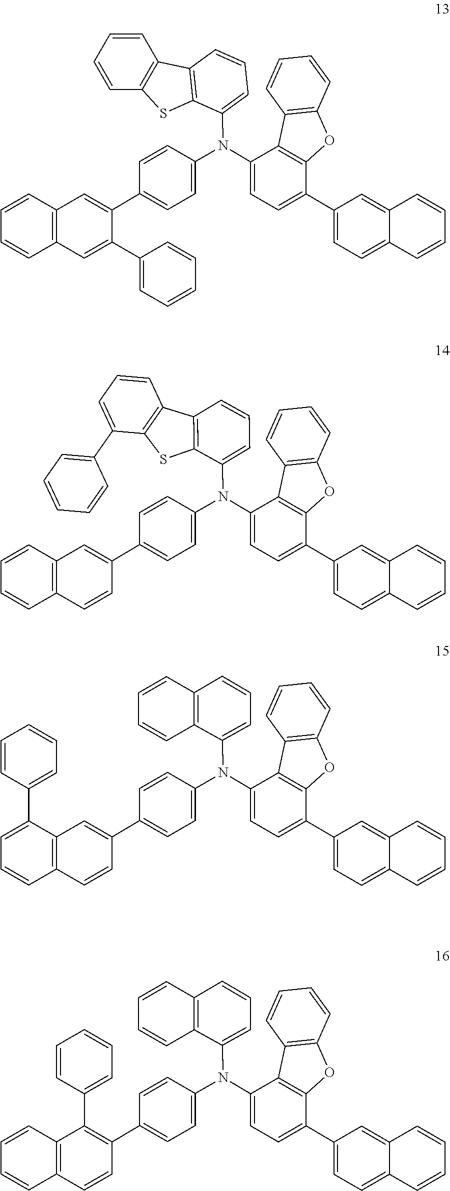 [SMILES: c1ccc(-c2cc3ccccc3cc2-c2ccc(N(c3cccc4c3sc3ccccc34)c3ccc(-c4ccc5ccccc5c4)c4oc5ccccc5c34)cc2)cc1 ; c1ccc(-c2cccc3c2sc2c(N(c4ccc(-c5ccc6ccccc6c5)cc4)c4ccc(-c5ccc6ccccc6c5)c5oc6ccccc6c45)cccc23)cc1 ; c1ccc(-c2cccc3ccc(-c4ccc(N(c5cccc6ccccc56)c5ccc(-c6ccc7ccccc7c6)c6oc7ccccc7c56)cc4)cc23)cc1 ; c1ccc(-c2c(-c3ccc(N(c4cccc5ccccc45)c4ccc(-c5ccc6ccccc6c5)c5oc6ccccc6c45)cc3)ccc3ccccc23)cc1]

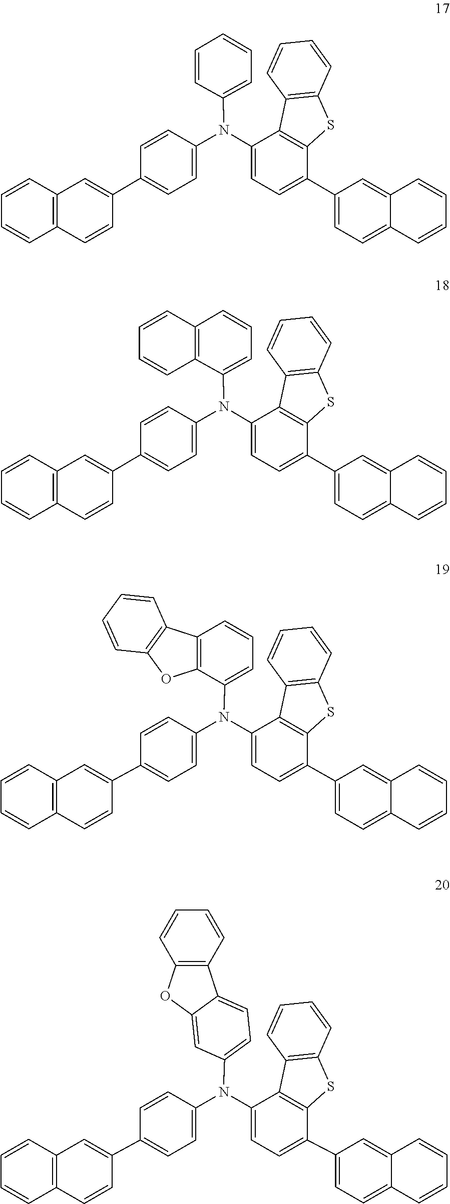 [SMILES: c1ccc(N(c2ccc(-c3ccc4ccccc4c3)cc2)c2ccc(-c3ccc4ccccc4c3)c3sc4ccccc4c23)cc1 ; c1ccc2cc(-c3ccc(N(c4cccc5ccccc45)c4ccc(-c5ccc6ccccc6c5)c5sc6ccccc6c45)cc3)ccc2c1 ; c1ccc2cc(-c3ccc(N(c4cccc5c4oc4ccccc45)c4ccc(-c5ccc6ccccc6c5)c5sc6ccccc6c45)cc3)ccc2c1 ; c1ccc2cc(-c3ccc(N(c4ccc5c(c4)oc4ccccc45)c4ccc(-c5ccc6ccccc6c5)c5sc6ccccc6c45)cc3)ccc2c1]

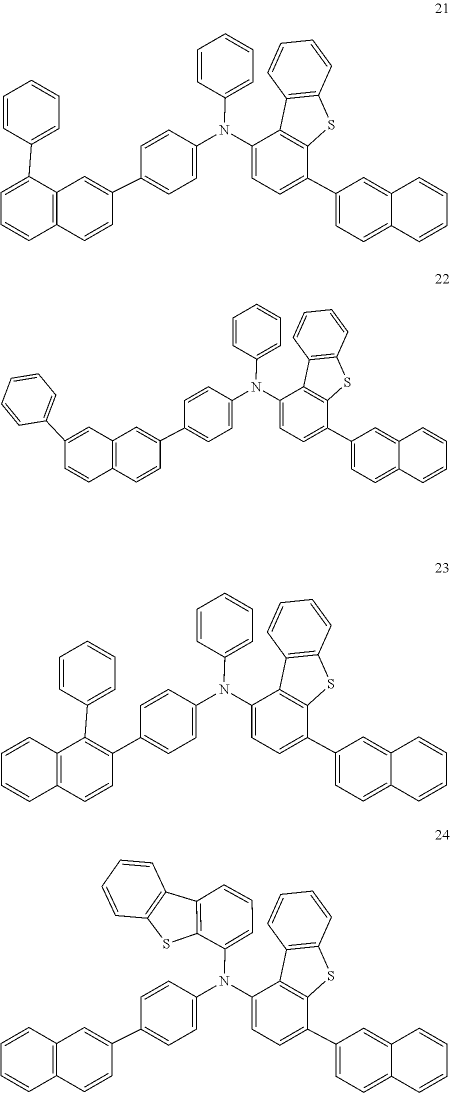 [SMILES: c1ccc(-c2cccc3ccc(-c4ccc(N(c5ccccc5)c5ccc(-c6ccc7ccccc7c6)c6sc7ccccc7c56)cc4)cc23)cc1 ; c1ccc(-c2ccc3ccc(-c4ccc(N(c5ccccc5)c5ccc(-c6ccc7ccccc7c6)c6sc7ccccc7c56)cc4)cc3c2)cc1 ; c1ccc(-c2c(-c3ccc(N(c4ccccc4)c4ccc(-c5ccc6ccccc6c5)c5sc6ccccc6c45)cc3)ccc3ccccc23)cc1 ; c1ccc2cc(-c3ccc(N(c4cccc5c4sc4ccccc45)c4ccc(-c5ccc6ccccc6c5)c5sc6ccccc6c45)cc3)ccc2c1]

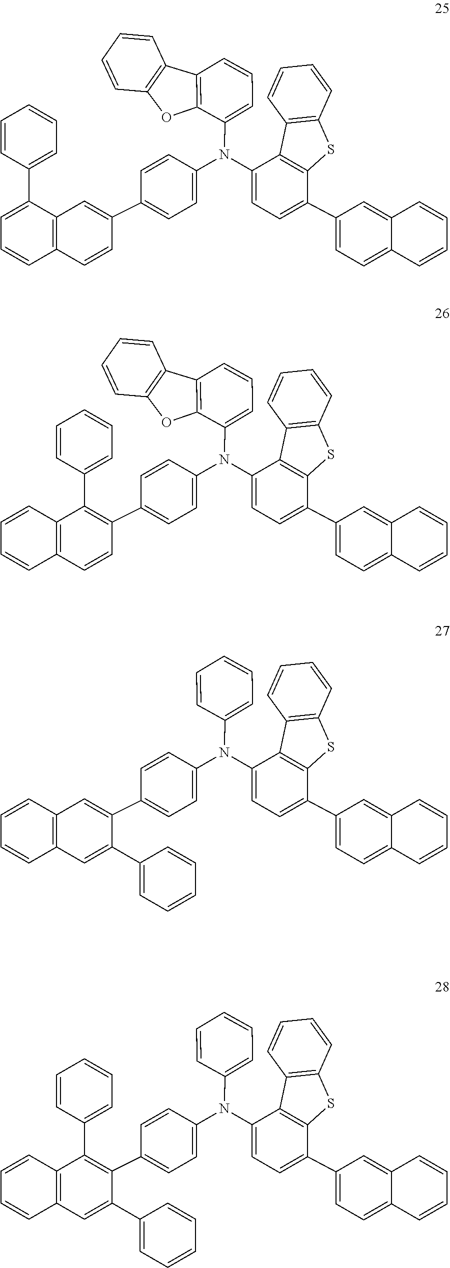 [SMILES: c1ccc(-c2cccc3ccc(-c4ccc(N(c5cccc6c5oc5ccccc56)c5ccc(-c6ccc7ccccc7c6)c6sc7ccccc7c56)cc4)cc23)cc1 ; c1ccc(-c2c(-c3ccc(N(c4cccc5c4oc4ccccc45)c4ccc(-c5ccc6ccccc6c5)c5sc6ccccc6c45)cc3)ccc3ccccc23)cc1 ; c1ccc(-c2cc3ccccc3cc2-c2ccc(N(c3ccccc3)c3ccc(-c4ccc5ccccc5c4)c4sc5ccccc5c34)cc2)cc1 ; c1ccc(-c2cc3ccccc3c(-c3ccccc3)c2-c2ccc(N(c3ccccc3)c3ccc(-c4ccc5ccccc5c4)c4sc5ccccc5c34)cc2)cc1]

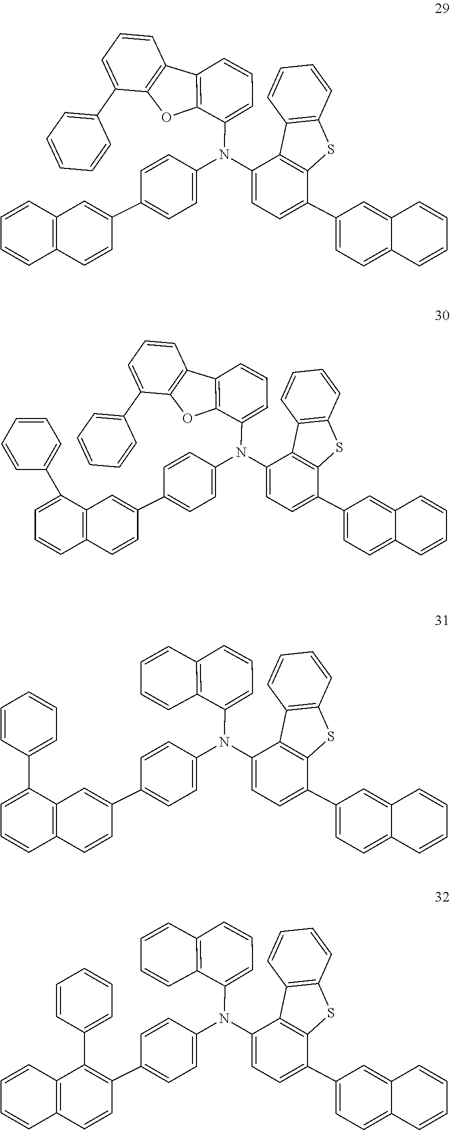 [SMILES: c1ccc(-c2cccc3c2oc2c(N(c4ccc(-c5ccc6ccccc6c5)cc4)c4ccc(-c5ccc6ccccc6c5)c5sc6ccccc6c45)cccc23)cc1 ; c1ccc(-c2cccc3ccc(-c4ccc(N(c5cccc6c5oc5c(-c7ccccc7)cccc56)c5ccc(-c6ccc7ccccc7c6)c6sc7ccccc7c56)cc4)cc23)cc1 ; c1ccc(-c2cccc3ccc(-c4ccc(N(c5cccc6ccccc56)c5ccc(-c6ccc7ccccc7c6)c6sc7ccccc7c56)cc4)cc23)cc1 ; c1ccc(-c2c(-c3ccc(N(c4cccc5ccccc45)c4ccc(-c5ccc6ccccc6c5)c5sc6ccccc6c45)cc3)ccc3ccccc23)cc1]

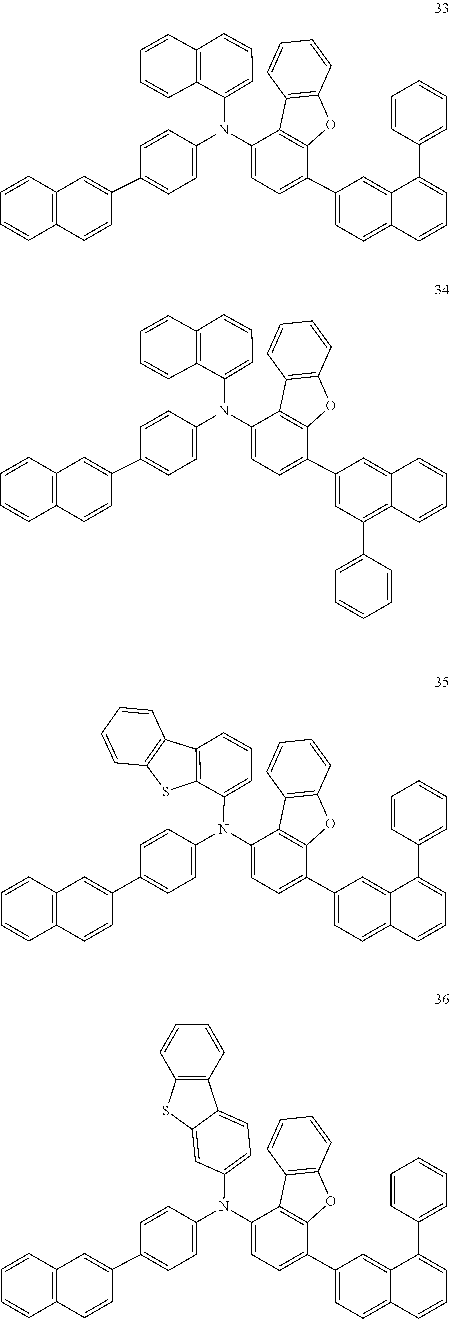 [SMILES: c1ccc(-c2cccc3ccc(-c4ccc(N(c5ccc(-c6ccc7ccccc7c6)cc5)c5cccc6ccccc56)c5c4oc4ccccc45)cc23)cc1 ; c1ccc(-c2cc(-c3ccc(N(c4ccc(-c5ccc6ccccc6c5)cc4)c4cccc5ccccc45)c4c3oc3ccccc34)cc3ccccc23)cc1 ; c1ccc(-c2cccc3ccc(-c4ccc(N(c5ccc(-c6ccc7ccccc7c6)cc5)c5cccc6c5sc5ccccc56)c5c4oc4ccccc45)cc23)cc1 ; c1ccc(-c2cccc3ccc(-c4ccc(N(c5ccc(-c6ccc7ccccc7c6)cc5)c5ccc6c(c5)sc5ccccc56)c5c4oc4ccccc45)cc23)cc1]

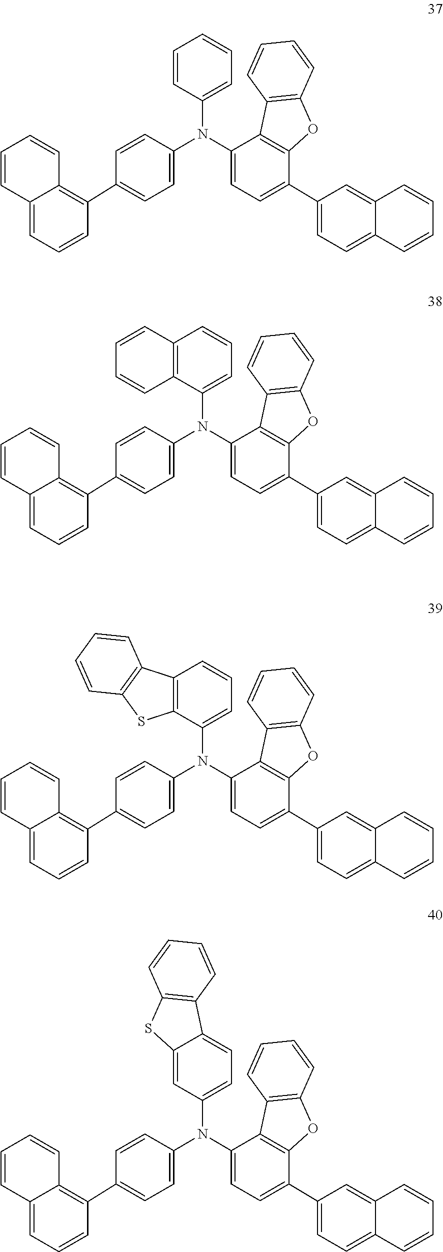 [SMILES: c1ccc(N(c2ccc(-c3cccc4ccccc34)cc2)c2ccc(-c3ccc4ccccc4c3)c3oc4ccccc4c23)cc1 ; c1ccc2cc(-c3ccc(N(c4ccc(-c5cccc6ccccc56)cc4)c4cccc5ccccc45)c4c3oc3ccccc34)ccc2c1 ; c1ccc2cc(-c3ccc(N(c4ccc(-c5cccc6ccccc56)cc4)c4cccc5c4sc4ccccc45)c4c3oc3ccccc34)ccc2c1 ; c1ccc2cc(-c3ccc(N(c4ccc(-c5cccc6ccccc56)cc4)c4ccc5c(c4)sc4ccccc45)c4c3oc3ccccc34)ccc2c1]

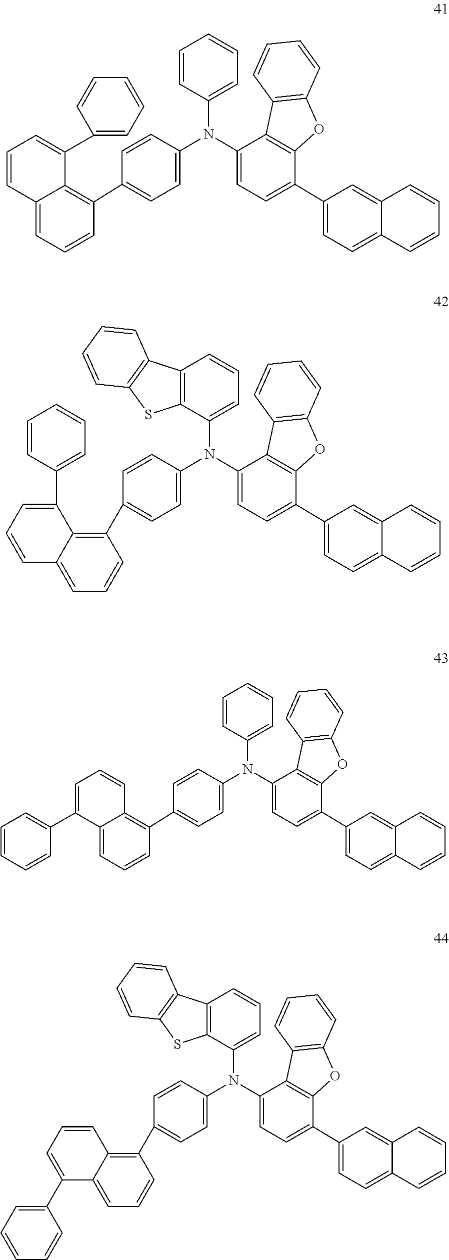 [SMILES: c1ccc(-c2cccc3cccc(-c4ccc(N(c5ccccc5)c5ccc(-c6ccc7ccccc7c6)c6oc7ccccc7c56)cc4)c23)cc1 ; c1ccc(-c2cccc3cccc(-c4ccc(N(c5cccc6c5sc5ccccc56)c5ccc(-c6ccc7ccccc7c6)c6oc7ccccc7c56)cc4)c23)cc1 ; c1ccc(-c2cccc3c(-c4ccc(N(c5ccccc5)c5ccc(-c6ccc7ccccc7c6)c6oc7ccccc7c56)cc4)cccc23)cc1 ; c1ccc(-c2cccc3c(-c4ccc(N(c5cccc6c5sc5ccccc56)c5ccc(-c6ccc7ccccc7c6)c6oc7ccccc7c56)cc4)cccc23)cc1]

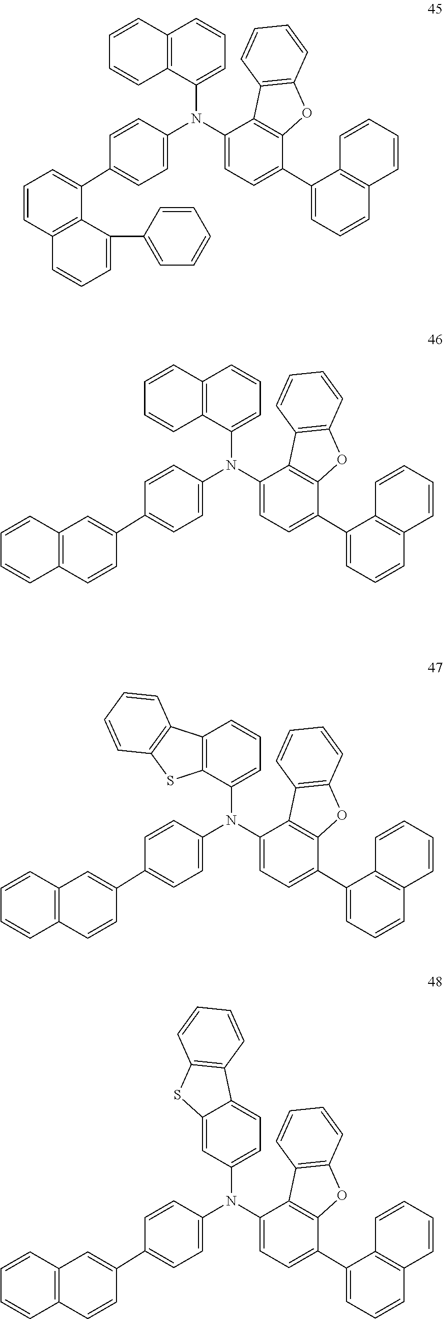 [SMILES: c1ccc(-c2cccc3cccc(-c4ccc(N(c5cccc6ccccc56)c5ccc(-c6cccc7ccccc67)c6oc7ccccc7c56)cc4)c23)cc1 ; c1ccc2cc(-c3ccc(N(c4cccc5ccccc45)c4ccc(-c5cccc6ccccc56)c5oc6ccccc6c45)cc3)ccc2c1 ; c1ccc2cc(-c3ccc(N(c4cccc5c4sc4ccccc45)c4ccc(-c5cccc6ccccc56)c5oc6ccccc6c45)cc3)ccc2c1 ; c1ccc2cc(-c3ccc(N(c4ccc5c(c4)sc4ccccc45)c4ccc(-c5cccc6ccccc56)c5oc6ccccc6c45)cc3)ccc2c1]

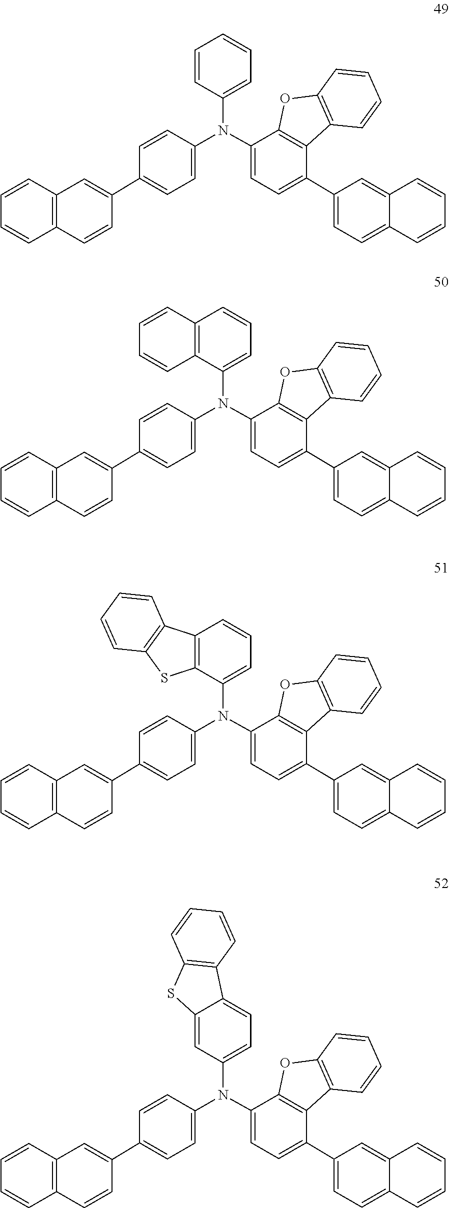 [SMILES: c1ccc(N(c2ccc(-c3ccc4ccccc4c3)cc2)c2ccc(-c3ccc4ccccc4c3)c3c2oc2ccccc23)cc1 ; c1ccc2cc(-c3ccc(N(c4cccc5ccccc45)c4ccc(-c5ccc6ccccc6c5)c5c4oc4ccccc45)cc3)ccc2c1 ; c1ccc2cc(-c3ccc(N(c4ccc(-c5ccc6ccccc6c5)c5c4oc4ccccc45)c4cccc5c4sc4ccccc45)cc3)ccc2c1 ; c1ccc2cc(-c3ccc(N(c4ccc5c(c4)sc4ccccc45)c4ccc(-c5ccc6ccccc6c5)c5c4oc4ccccc45)cc3)ccc2c1]

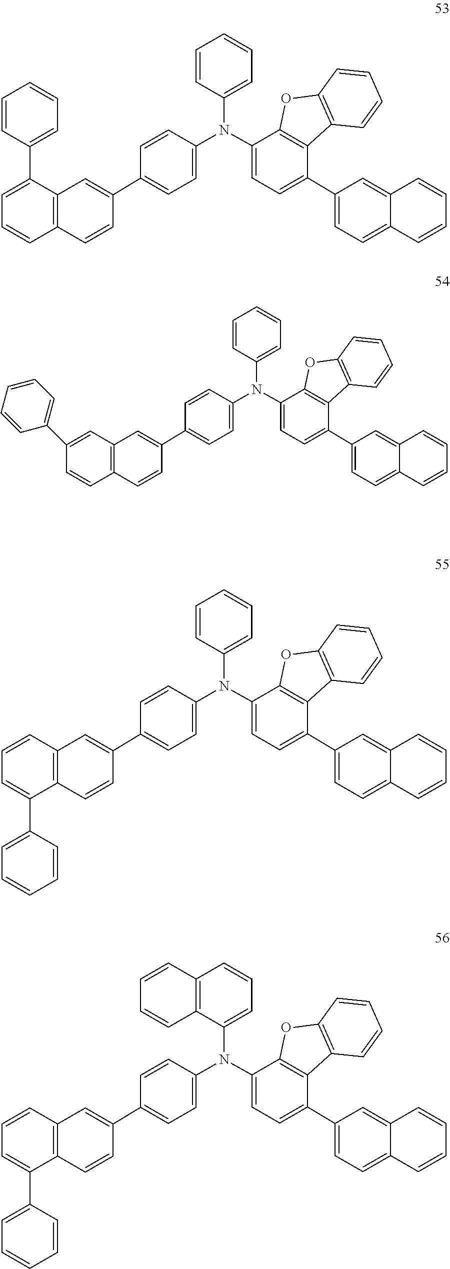 [SMILES: c1ccc(-c2cccc3ccc(-c4ccc(N(c5ccccc5)c5ccc(-c6ccc7ccccc7c6)c6c5oc5ccccc56)cc4)cc23)cc1 ; c1ccc(-c2ccc3ccc(-c4ccc(N(c5ccccc5)c5ccc(-c6ccc7ccccc7c6)c6c5oc5ccccc56)cc4)cc3c2)cc1 ; c1ccc(-c2cccc3cc(-c4ccc(N(c5ccccc5)c5ccc(-c6ccc7ccccc7c6)c6c5oc5ccccc56)cc4)ccc23)cc1 ; c1ccc(-c2cccc3cc(-c4ccc(N(c5cccc6ccccc56)c5ccc(-c6ccc7ccccc7c6)c6c5oc5ccccc56)cc4)ccc23)cc1]

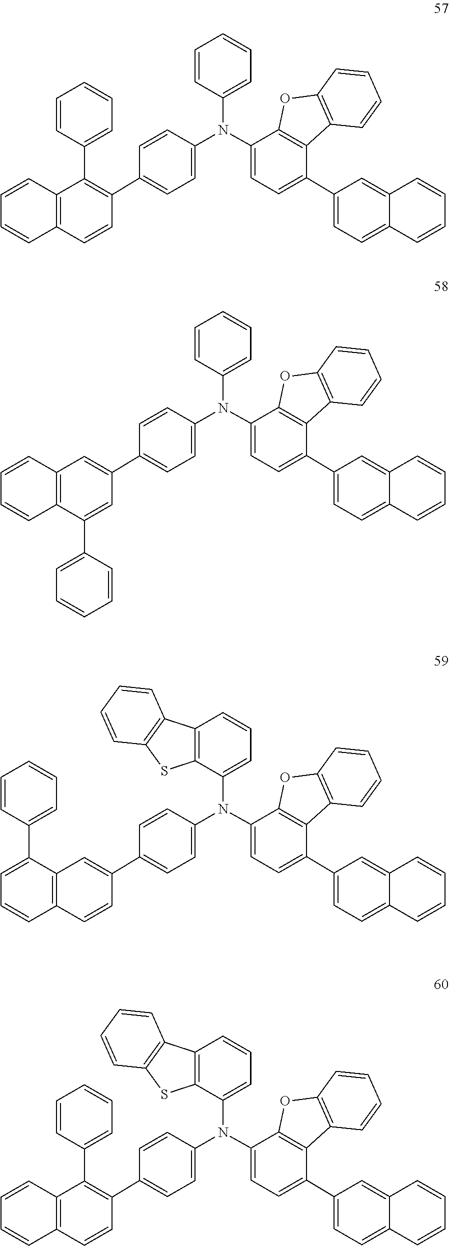 [SMILES: c1ccc(-c2c(-c3ccc(N(c4ccccc4)c4ccc(-c5ccc6ccccc6c5)c5c4oc4ccccc45)cc3)ccc3ccccc23)cc1 ; c1ccc(-c2cc(-c3ccc(N(c4ccccc4)c4ccc(-c5ccc6ccccc6c5)c5c4oc4ccccc45)cc3)cc3ccccc23)cc1 ; c1ccc(-c2cccc3ccc(-c4ccc(N(c5ccc(-c6ccc7ccccc7c6)c6c5oc5ccccc56)c5cccc6c5sc5ccccc56)cc4)cc23)cc1 ; c1ccc(-c2c(-c3ccc(N(c4ccc(-c5ccc6ccccc6c5)c5c4oc4ccccc45)c4cccc5c4sc4ccccc45)cc3)ccc3ccccc23)cc1]

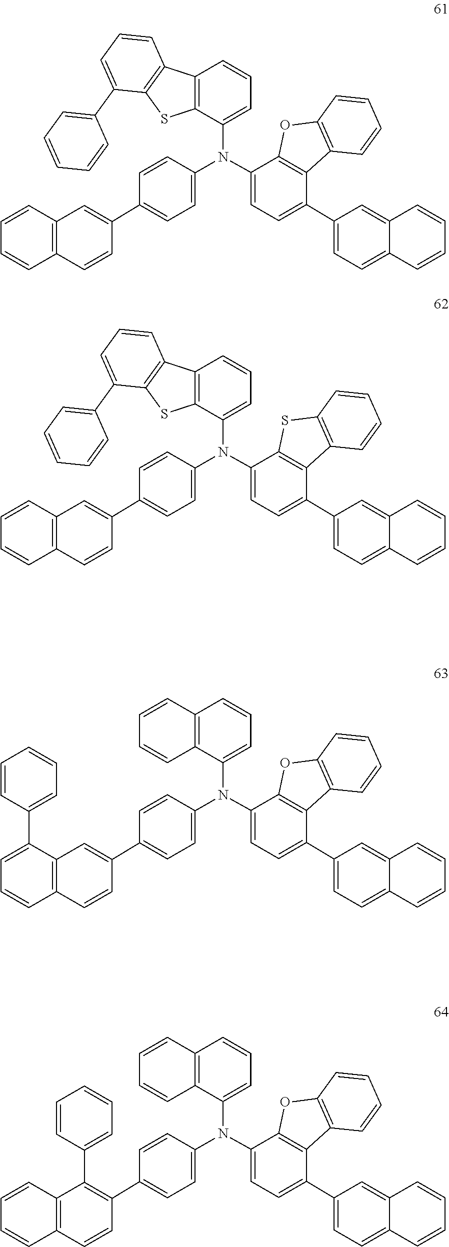 [SMILES: c1ccc(-c2cccc3c2sc2c(N(c4ccc(-c5ccc6ccccc6c5)cc4)c4ccc(-c5ccc6ccccc6c5)c5c4oc4ccccc45)cccc23)cc1 ; c1ccc(-c2cccc3c2sc2c(N(c4ccc(-c5ccc6ccccc6c5)cc4)c4ccc(-c5ccc6ccccc6c5)c5c4sc4ccccc45)cccc23)cc1 ; c1ccc(-c2cccc3ccc(-c4ccc(N(c5cccc6ccccc56)c5ccc(-c6ccc7ccccc7c6)c6c5oc5ccccc56)cc4)cc23)cc1 ; c1ccc(-c2c(-c3ccc(N(c4cccc5ccccc45)c4ccc(-c5ccc6ccccc6c5)c5c4oc4ccccc45)cc3)ccc3ccccc23)cc1]

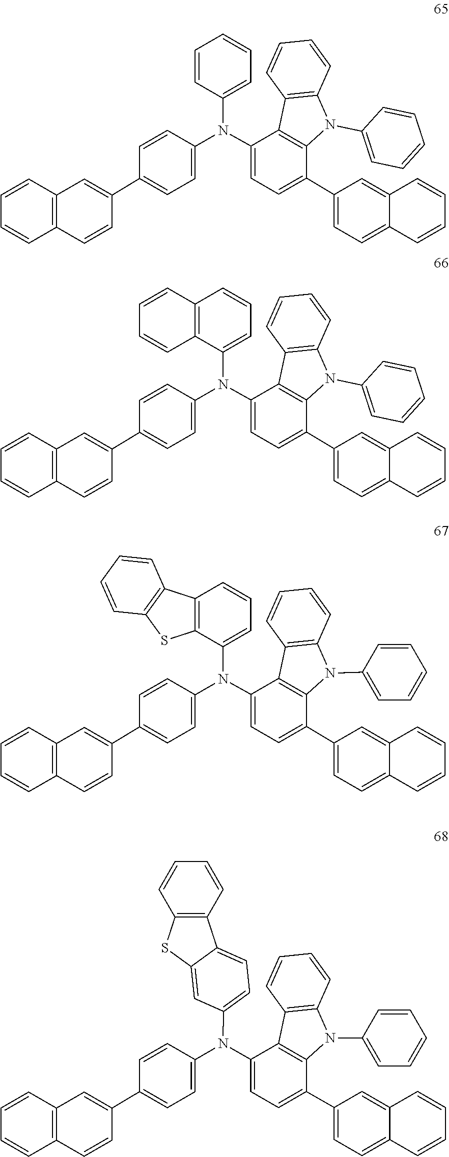 [SMILES: c1ccc(N(c2ccc(-c3ccc4ccccc4c3)cc2)c2ccc(-c3ccc4ccccc4c3)c3c2c2ccccc2n3-c2ccccc2)cc1 ; c1ccc(-n2c3ccccc3c3c(N(c4ccc(-c5ccc6ccccc6c5)cc4)c4cccc5ccccc45)ccc(-c4ccc5ccccc5c4)c32)cc1 ; c1ccc(-n2c3ccccc3c3c(N(c4ccc(-c5ccc6ccccc6c5)cc4)c4cccc5c4sc4ccccc45)ccc(-c4ccc5ccccc5c4)c32)cc1 ; c1ccc(-n2c3ccccc3c3c(N(c4ccc(-c5ccc6ccccc6c5)cc4)c4ccc5c(c4)sc4ccccc45)ccc(-c4ccc5ccccc5c4)c32)cc1]

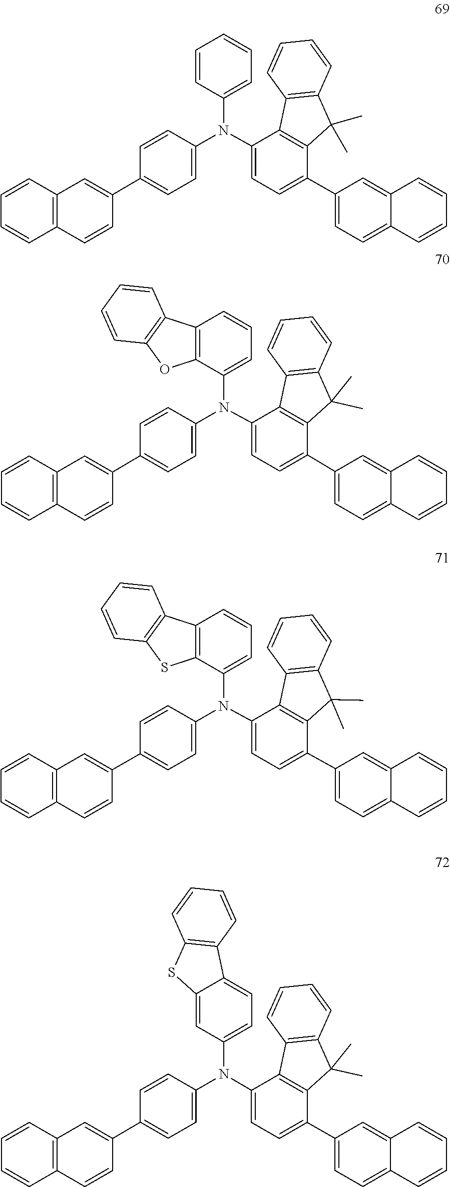 [SMILES: CC1(C)c2ccccc2-c2c(N(c3ccccc3)c3ccc(-c4ccc5ccccc5c4)cc3)ccc(-c3ccc4ccccc4c3)c21 ; CC1(C)c2ccccc2-c2c(N(c3ccc(-c4ccc5ccccc5c4)cc3)c3cccc4c3oc3ccccc34)ccc(-c3ccc4ccccc4c3)c21 ; CC1(C)c2ccccc2-c2c(N(c3ccc(-c4ccc5ccccc5c4)cc3)c3cccc4c3sc3ccccc34)ccc(-c3ccc4ccccc4c3)c21 ; CC1(C)c2ccccc2-c2c(N(c3ccc(-c4ccc5ccccc5c4)cc3)c3ccc4c(c3)sc3ccccc34)ccc(-c3ccc4ccccc4c3)c21]

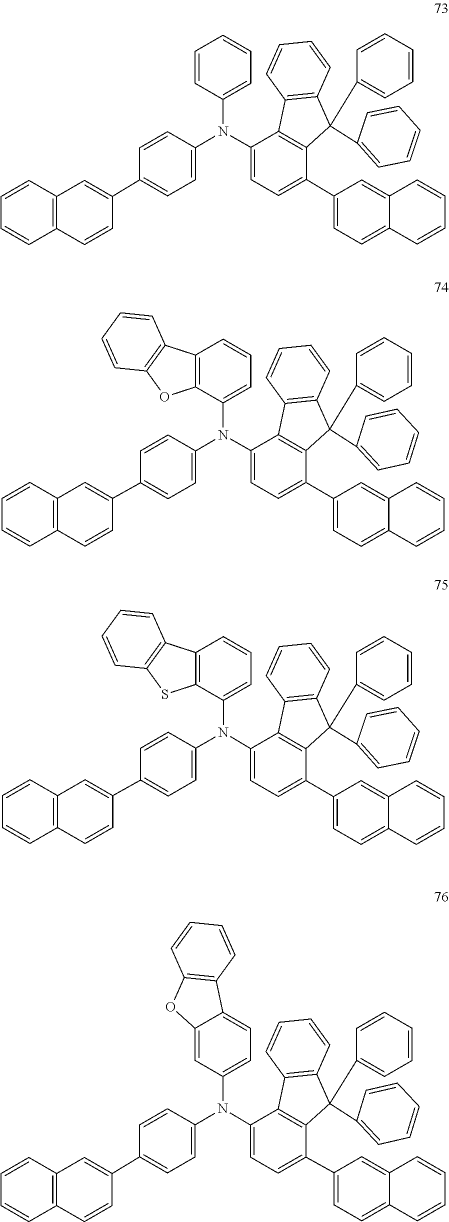 [SMILES: c1ccc(N(c2ccc(-c3ccc4ccccc4c3)cc2)c2ccc(-c3ccc4ccccc4c3)c3c2-c2ccccc2C3(c2ccccc2)c2ccccc2)cc1 ; c1ccc(C2(c3ccccc3)c3ccccc3-c3c(N(c4ccc(-c5ccc6ccccc6c5)cc4)c4cccc5c4oc4ccccc45)ccc(-c4ccc5ccccc5c4)c32)cc1 ; c1ccc(C2(c3ccccc3)c3ccccc3-c3c(N(c4ccc(-c5ccc6ccccc6c5)cc4)c4cccc5c4sc4ccccc45)ccc(-c4ccc5ccccc5c4)c32)cc1 ; c1ccc(C2(c3ccccc3)c3ccccc3-c3c(N(c4ccc(-c5ccc6ccccc6c5)cc4)c4ccc5c(c4)oc4ccccc45)ccc(-c4ccc5ccccc5c4)c32)cc1]

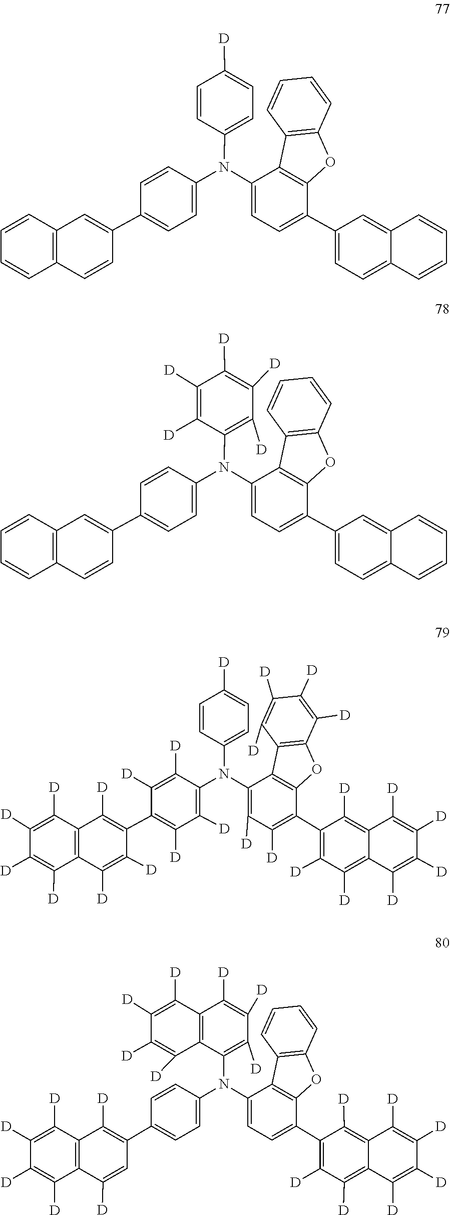

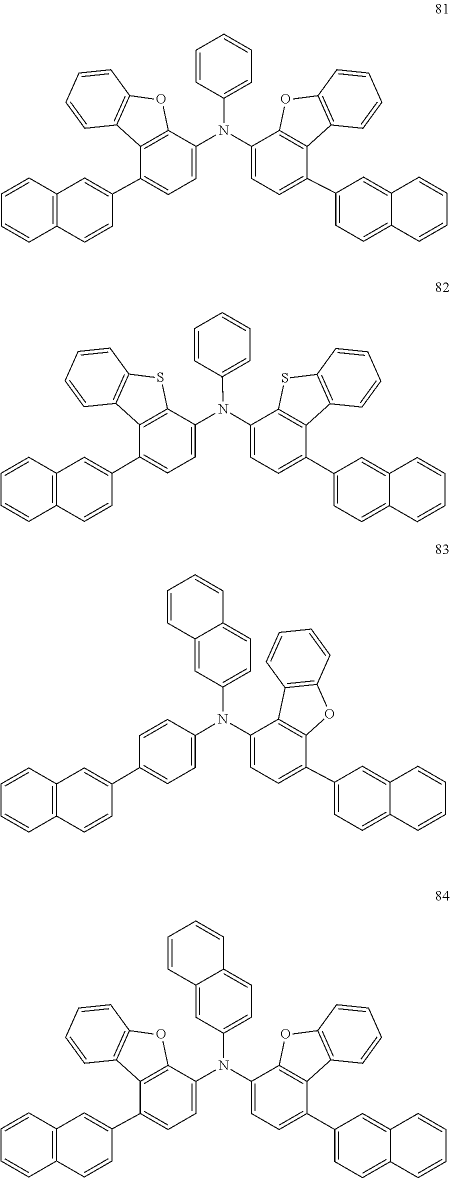 [SMILES: c1ccc(N(c2ccc(-c3ccc4ccccc4c3)c3c2oc2ccccc23)c2ccc(-c3ccc4ccccc4c3)c3c2oc2ccccc23)cc1 ; c1ccc(N(c2ccc(-c3ccc4ccccc4c3)c3c2sc2ccccc23)c2ccc(-c3ccc4ccccc4c3)c3c2sc2ccccc23)cc1 ; c1ccc2cc(-c3ccc(N(c4ccc5ccccc5c4)c4ccc(-c5ccc6ccccc6c5)c5oc6ccccc6c45)cc3)ccc2c1 ; c1ccc2cc(-c3ccc(N(c4ccc5ccccc5c4)c4ccc(-c5ccc6ccccc6c5)c5c4oc4ccccc45)c4oc5ccccc5c34)ccc2c1]

The amine compound according to an embodiment represented by Formula 1 and Formula 2 may include at least one dibenzoheterole moiety substituted with a naphthyl group. In the amine compound according to an embodiment, the dibenzoheterole moiety may be protected by a naphthyl group having excellent heat and charge resistance to prevent deterioration, and thus the stability of the amine compound according to an embodiment may be improved. The lifetime of the light-emitting element according to an embodiment including the amine compound according to an embodiment may also be improved. Since the amine compound according to an embodiment may include at least one dibenzoheterole moiety having a naphthyl group as a substituent, the symmetry of the molecule is lowered and the crystallinity is thus suppressed, thereby improving hole transport properties when used in the light-emitting element. Accordingly, the light-emitting efficiency of the light-emitting element according to an embodiment including the amine compound according to an embodiment may also be improved.

When the light-emitting element ED according to an embodiment includes hole transport layers HTL1 and HTL2, the second hole transport layer HTL2 adjacent to the light-emitting layer EML may include the amine compound of the above-described embodiment. The first hole transport layer HTL1 disposed under the second hole transport layer HTL2 and adjacent to the first electrode EL1 may include an amine derivative compound represented by Formula 3.

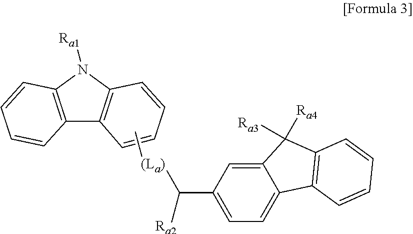

In Formula 3, Lₐ may be a direct linkage, a substituted or unsubstituted arylene group having 6 to 30 ring-forming carbon atoms, or a substituted or unsubstituted heteroarylene group having 2 to 30 ring-forming carbon atoms. In Formula 3, Rₐ₁ to Rₐ₄ may each independently be a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, or may be bonded with an adjacent group to form a ring.

For example, Lₐ may be a direct linkage or a substituted or unsubstituted phenylene group. For example, Rₐ₁ may be a substituted or unsubstituted phenyl group. However, embodiments are not limited thereto.

In the amine derivative compound represented by Formula 3, Rₐ₂ may be an aryl group or a heteroaryl group. For example, Rₐ₂ may be a substituted or unsubstituted phenyl group, a substituted or unsubstituted naphthyl group, a substituted or unsubstituted biphenyl group, a substituted or unsubstituted dibenzofuran group, or a substituted or unsubstituted dibenzothiophene group.

The amine derivative compound represented by Formula 3 may be represented by Compound HT1. In an embodiment, the first hole transport layer HTL1 may include Compound HT1.

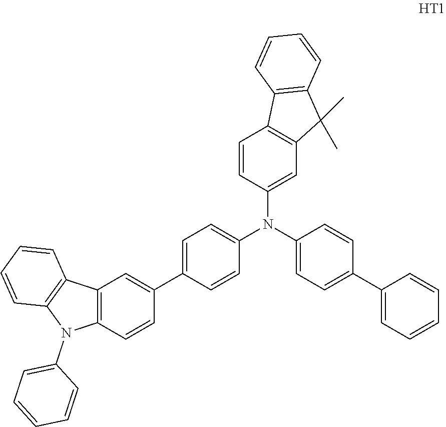 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21]

The light-emitting element ED according to an embodiment may further include, in the hole transport region HTR, a material for the hole transport region in addition to the amine compound according to an embodiment and the amine derivative compound represented by Formula 3.

The hole transport region HTR may include a compound represented by Formula H-1.

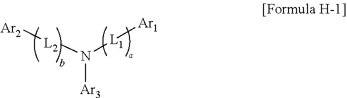

In Formula H-1, L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms. In Formula H-1, a and b may each independently be an integer from 0 to 10. When a or b is 2 or more, multiple L₁ groups and multiple L₂ groups may each independently be a substituted or unsubstituted arylene group having 6 to 30 ring-forming carbon atoms, or a substituted or unsubstituted heteroarylene group having 2 to 30 ring-forming carbon atoms.

In Formula H-1, Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. In Formula H-1, Ar₃ may be a substituted or unsubstituted aryl group having 6 to 30 ring-forming carbon atoms.

In an embodiment, the compound represented by Formula H-1 may be a monoamine compound. In another embodiment, the compound represented by Formula H-1 may be a diamine compound in which at least one of Ar₁ to Ar₃ includes an amine group as a substituent. In still another embodiment, the compound represented by Formula H-1 may be a carbazole-based compound in which at least one of Ar₁ and Ar₂ includes a substituted or unsubstituted carbazole group, or may be a fluorene-based compound in which at least one among Ar₁ and Ar₂ includes a substituted or unsubstituted fluorene group.

The compound represented by Formula H-1 may be any one selected from Compound Group H. However, the compounds listed in Compound Group H are only examples, and the compound represented by Formula H-1 is not limited to Compound Group H.

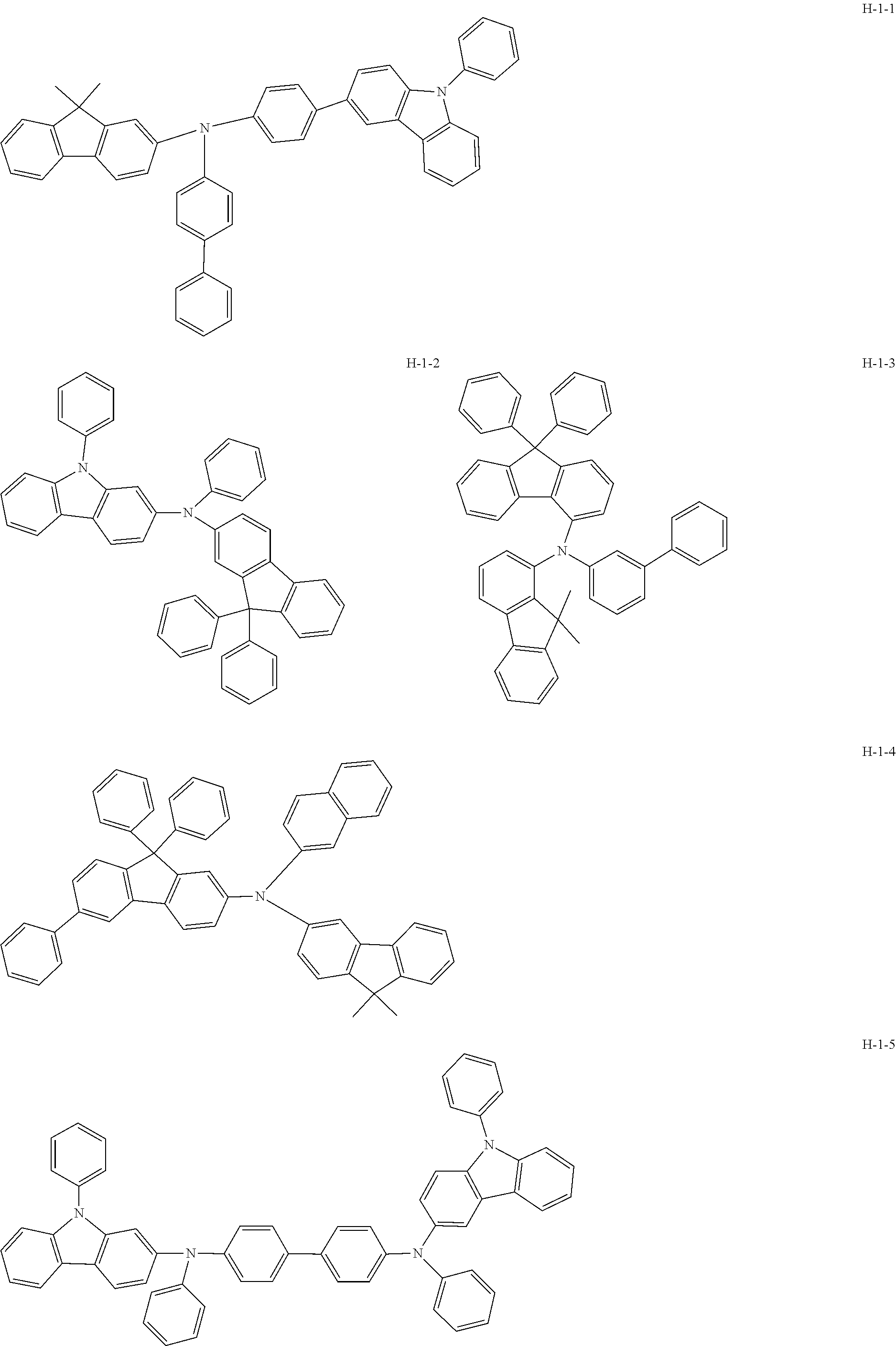 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21 ; CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21 ; c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1]

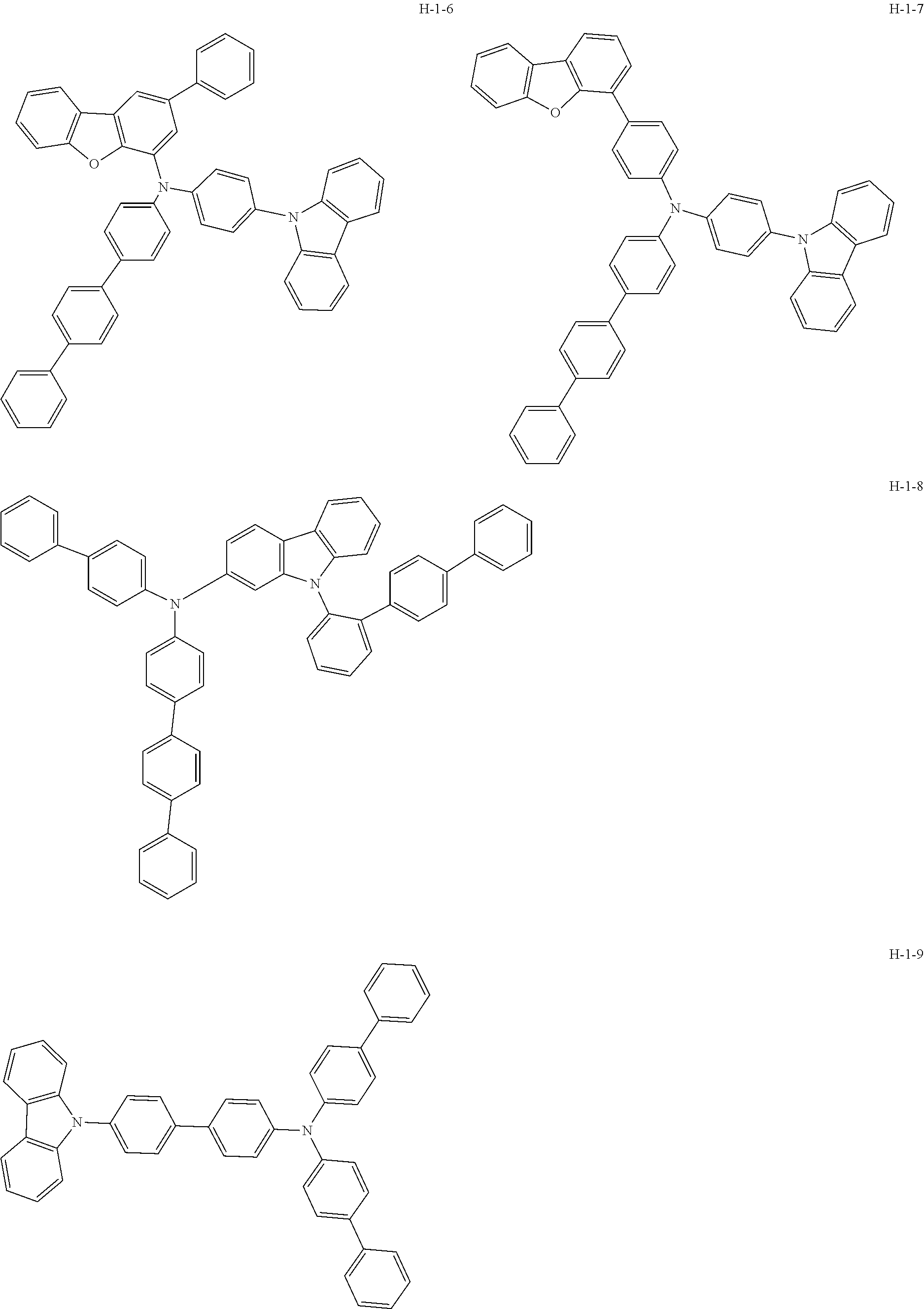 [SMILES: c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1 ; c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

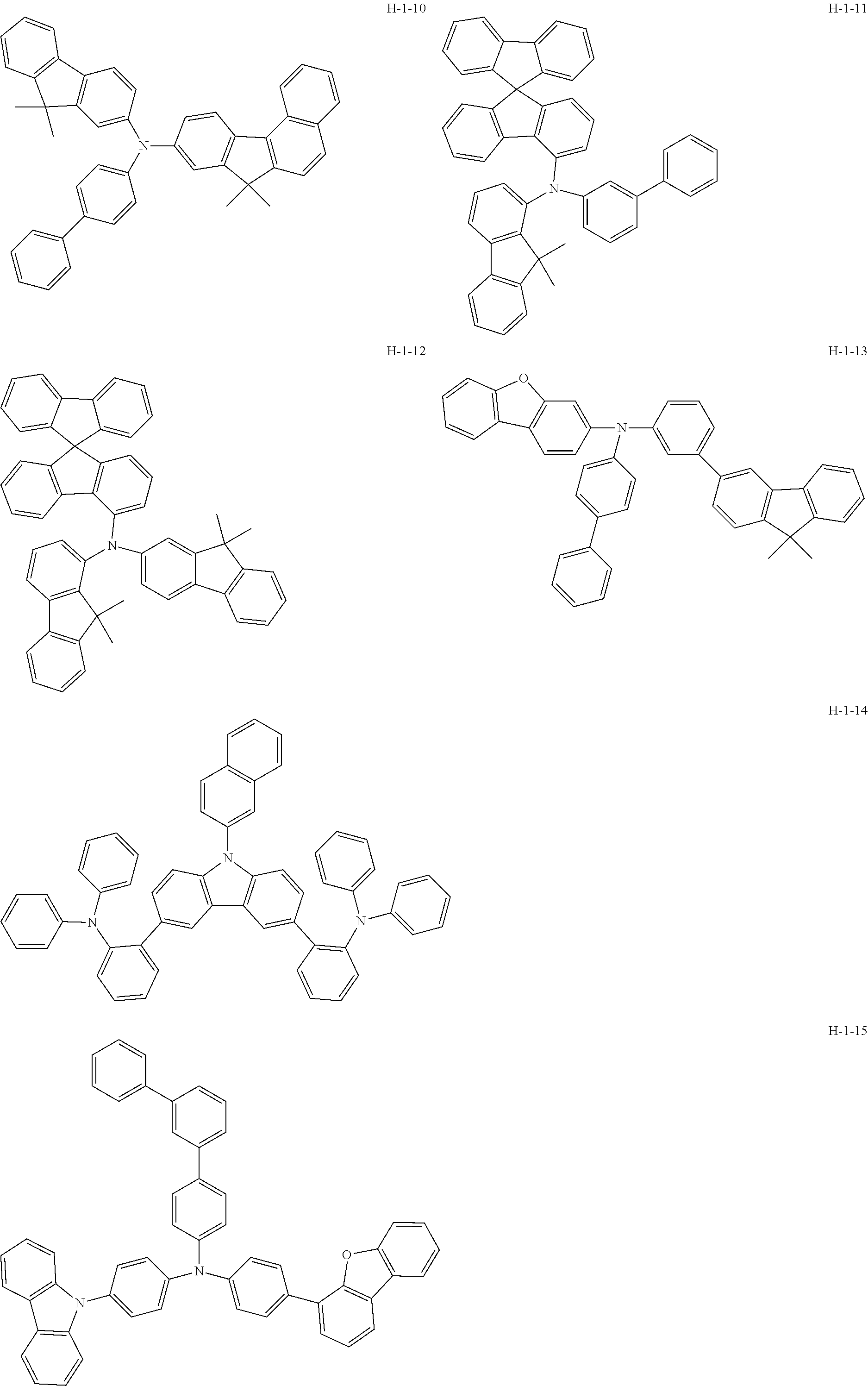

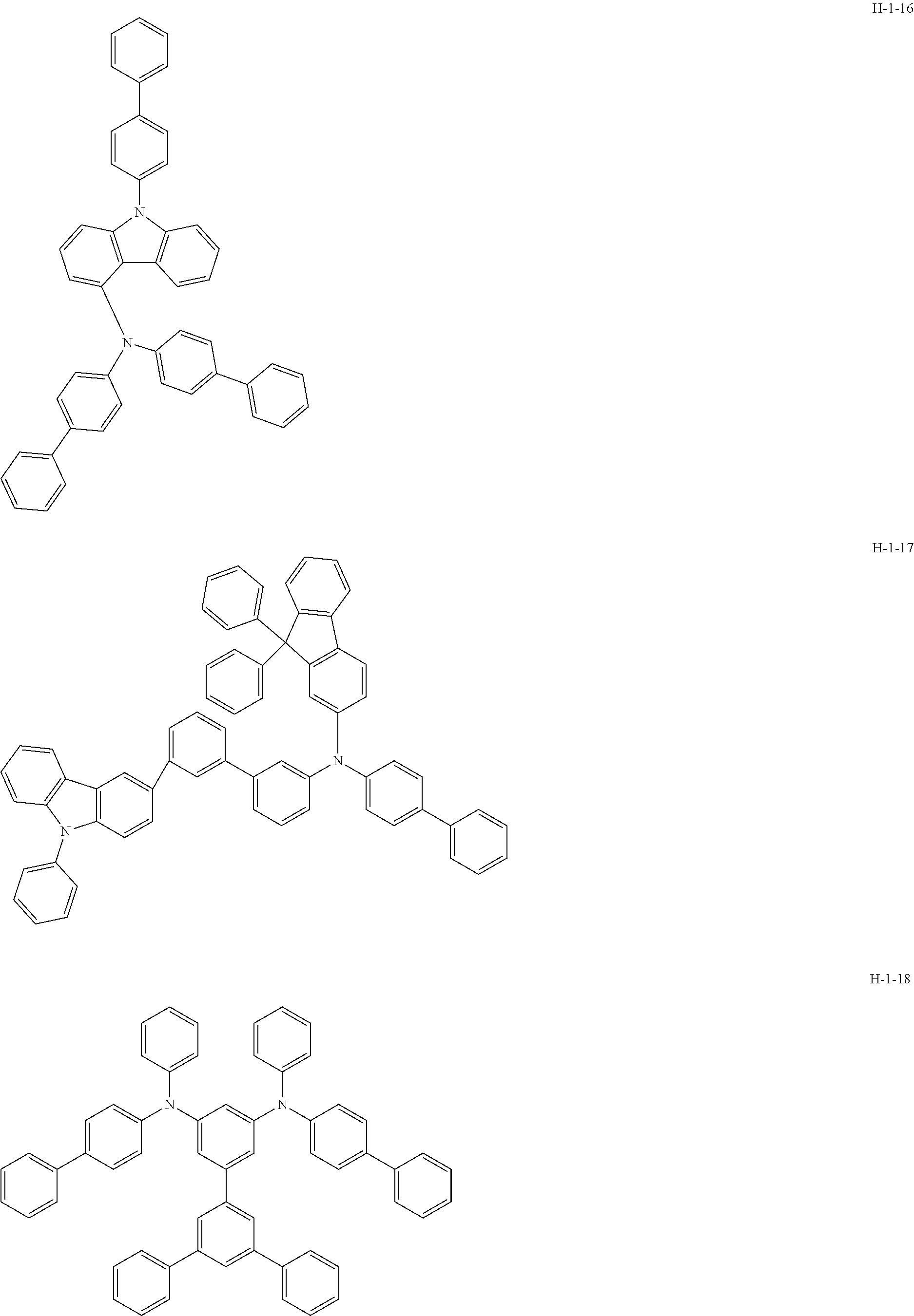 [SMILES: c1ccc(-c2ccc(N(c3ccc(-c4ccccc4)cc3)c3cccc4c3c3ccccc3n4-c3ccc(-c4ccccc4)cc3)cc2)cc1 ; c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1]

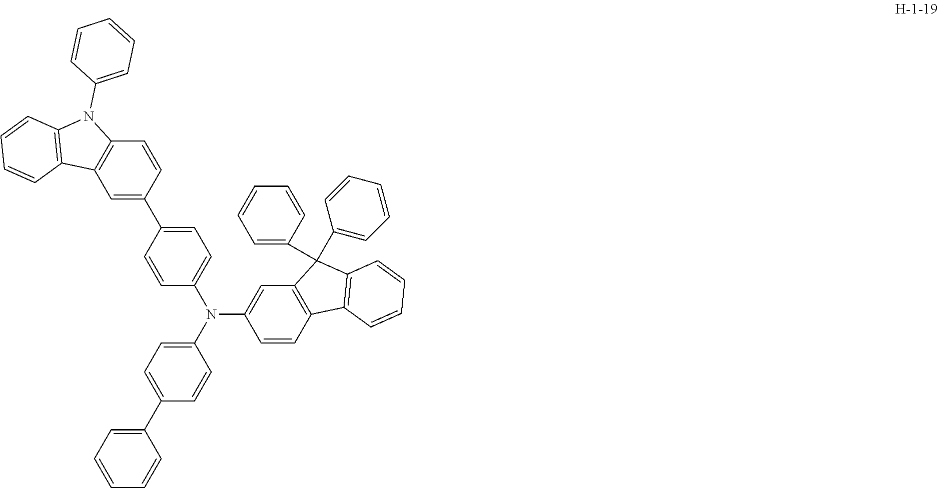 [SMILES: c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

The hole transport region HTR may include a phthalocyanine compound such as copper phthalocyanine, N1,N1′-([1,1′-biphenyl]-4,4′-diyl)bis(N1-phenyl-N4,N4-di-m-tolylbenzene-1,4-diamine (DNTPD), 4,4′,4″-[tris(3-methylphenyl)phenylamino] triphenylamine (m-MTDATA), 4,4′4″-tris(N,N-diphenylamino)triphenylamine (TDATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(naphthalene-1-yl)-N,N′-diphenyl-benzidine (NPB or NPD), triphenylamine-containing polyetherketone (TPAPEK), 4-isopropyl-4′-methyldiphenyliodonium [tetrakis(pentafluorophenyl)borate], dipyrazino[2,3-f:2′,3′-h] quinoxaline-2,3,6,7,10,11-hexacarbonitrile (HATCN), etc.

The hole transport region HTR may include carbazole derivatives such as N-phenyl carbazole and polyvinyl carbazole, fluorene-based derivatives, N,N′-bis(3-methylphenyl)-N,N′-diphenyl-[1,1′-biphenyl]-4,4′-diamine (TPD), triphenylamine derivatives such as 4,4′,4″-tris(N-carbazolyl)triphenylamine (TCTA), N,N′-di(naphthalene-1-yl)-N,N′-diphenyl-benzidine (NPB), 4,4′-cyclohexylidene bis[N,N-bis(4-methylphenyl)benzenamine] (TAPC), 4,4′-bis[N,N′-(3-tolyl)amino]-3,3′-dimethylbiphenyl (HMTPD), 1,3-bis(N-carbazolyl)benzene (mCP), etc.

The hole transport region HTR may include 9-(4-tert-butylphenyl)-3,6-bis(triphenylsilyl)-9H-carbazole (CzSi), 9-phenyl-9H-3,9′-bicarbazole (CCP), or 1,3-bis(1,8-dimethyl-9H-carbazol-9-yl)benzene (mDCP), etc.

The hole transport region HTR may include the above-described compounds of the hole transport region in at least one of a hole injection layer HIL, a hole transport layer HTL, or an electron blocking layer EBL.

The hole transport region HTR may have a thickness in a range of about 100 Å to about 10,000 Å. For example, the hole transport region HTR may have a thickness in a range of about 100 Å to about 5,000 Å. When the hole transport region HTR includes a hole injection layer HIL, the hole injection layer HIL may have a thickness, for example, in a range of about 30 Å to about 1,000 Å. When the hole transport region HTR includes a hole transport layer HTL, the hole transport layer HTL may have a thickness in a range of about 30 Å to about 1,000 Å. When the hole transport region HTR includes an electron blocking layer EBL, the electron blocking layer EBL may have a thickness in a range of about 10 Å to about 1,000 Å. When the thicknesses of the hole transport region HTR, the hole injection layer HIL, the hole transport layer HTL and the electron blocking layer EBL satisfy the above-described ranges, satisfactory hole transport properties may be achieved without a substantial increase in driving voltage.

The hole transport region HTR may further include a charge generating material to increase conductivity, in addition to the above-described materials. The charge generating material may be dispersed uniformly or non-uniformly in the hole transport region HTR. The charge generating material may be, for example, a p-dopant. The p-dopant may include at least one of a metal halide compound, a quinone derivative, a metal oxide, or a cyano group-containing compound, but embodiments are not limited thereto. For example, the p-dopant may include a metal halide compound such as CuI and RbI, a quinone derivative such as tetracyanoquinodimethane (TCNQ) and 2,3,5,6-tetrafluoro-7,7,8,8-tetracyanoquinodimethane (F4-TCNQ), a metal oxide such as tungsten oxide and molybdenum oxide, a cyano group-containing compound such as dipyrazino[2,3-f: 2′,3′-h] quinoxaline-2,3,6,7,10,11-hexacarbonitrile (HATCN) and 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), etc., but embodiments are not limited thereto.

As described above, the hole transport region HTR may further include at least one of a buffer layer (not shown) or an electron blocking layer EBL, in addition to the hole injection layer HIL and the hole transport layer HTL. The buffer layer (not shown) may compensate for a resonance distance according to a wavelength of light emitted from the light-emitting layer EML and may increase light-emitting efficiency. As materials included in the buffer layer (not shown), materials which may be included in the hole transport region HTR may be used. The electron blocking layer EBL may prevent the injection of electrons from the electron transport region ETR to the hole transport region HTR.

The light-emitting layer EML is provided on the hole transport region HTR. The light-emitting layer EML may have a thickness, for example, in a range of about 100 Å to about 1,000 Å. For example, the light-emitting layer EML may have a thickness in a range of about 100 Å to about 300 Å. The light-emitting layer EML may be a layer formed of a single material, a layer formed of different materials, or a structure having multiple layers formed of different materials.

In the light-emitting element ED according to an embodiment, the light-emitting layer EML may emit blue light. The light-emitting element ED according to an embodiment may include above-described amine compound according to an embodiment in the hole transport region HTR to exhibit high efficiency and long lifetime characteristics in the blue light-emitting region. However, embodiments are limited thereto.

In the light-emitting element ED according to an embodiment, the light-emitting layer EML may include an anthracene derivative, a pyrene derivative, a fluoranthene derivative, a chrysene derivative, a dihydrobenzanthracene derivative, or a triphenylene derivative. For example, the light-emitting layer EML may include an anthracene derivative or a pyrene derivative.

In the light-emitting element ED according to an embodiment illustrated in FIGS. 3 to 7, the light-emitting layer EML may include a host and a dopant, and the light-emitting layer EML may include a compound represented by Formula E-1. The compound represented by Formula E-1 may be used as a fluorescence host material.

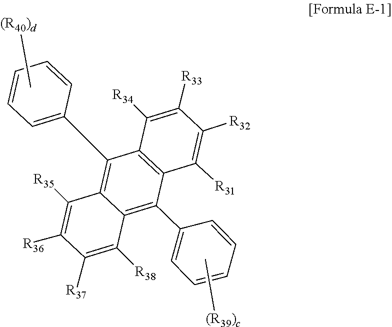

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. For example, R₃₁ to R₄₀ may be bonded to an adjacent group to form a saturated hydrocarbon ring, an unsaturated hydrocarbon ring, a saturated heterocycle, or an unsaturated heterocycle.

In Formula E-1, c and d may each independently be an integer from 0 to 5.

The compound represented by Formula E-1 may be any one selected from Compound E1 to Compound E19:

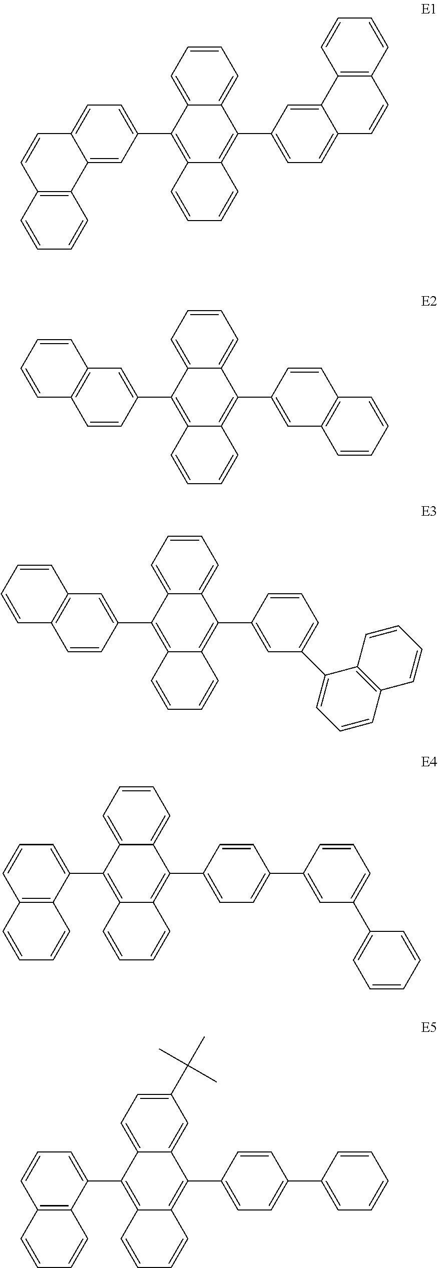

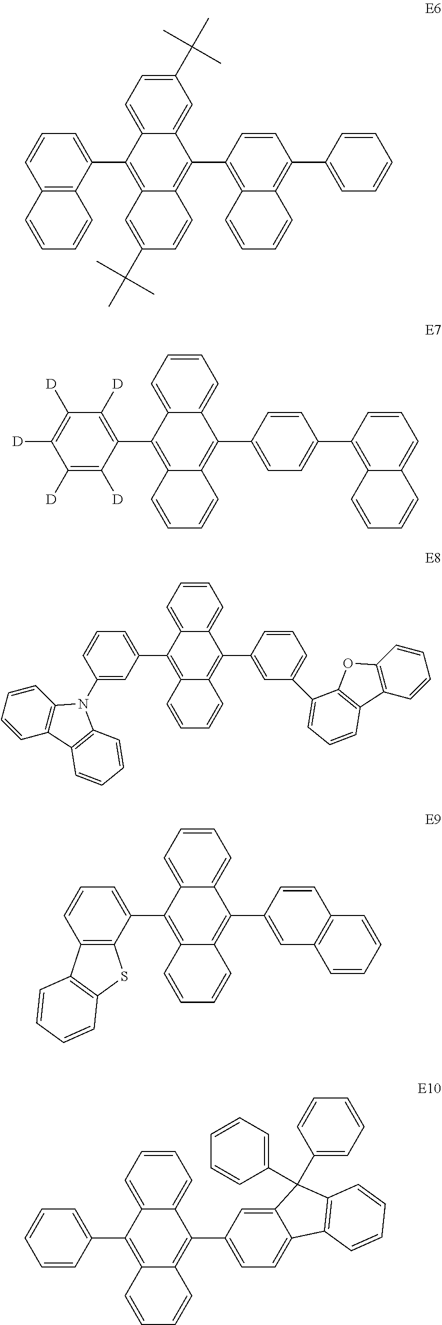

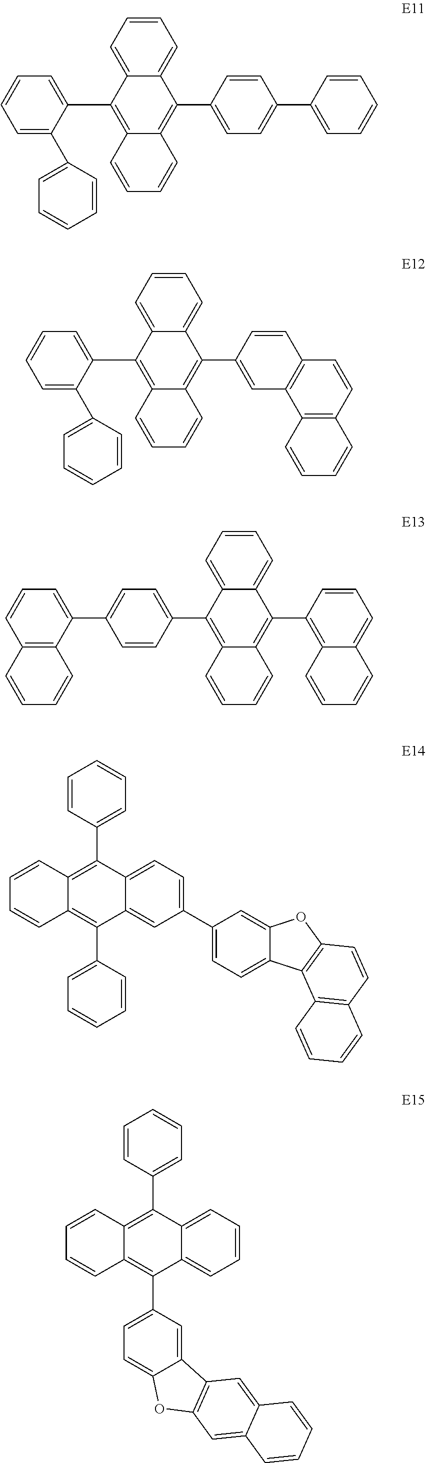

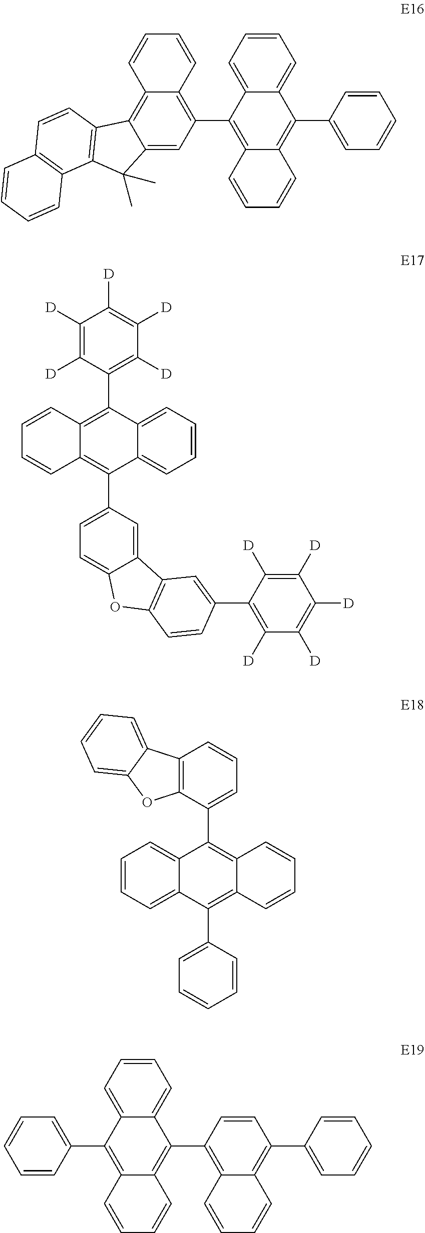

In an embodiment, the light-emitting layer EML may include a compound represented by Formula E-2a or Formula E-2b. The compound represented by Formula E-2a or Formula E-2b may be used as a phosphorescence host material.

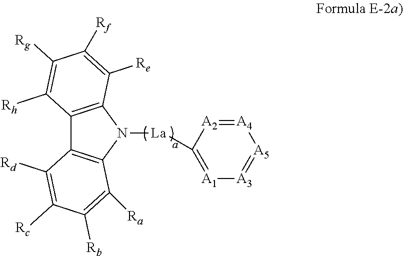

In Formula E-2a, a may be an integer from 0 to 10, and Lₐ may be a direct linkage, a substituted or unsubstituted arylene group having 6 to 30 ring-forming carbon atoms, or a substituted or unsubstituted heteroarylene group having 2 to 30 ring-forming carbon atoms. When a is 2 or more, multiple Lₐ groups may each independently be a substituted or unsubstituted arylene group having 6 to 30 ring-forming carbon atoms, or a substituted or unsubstituted heteroarylene group having 2 to 30 ring-forming carbon atoms.

In Formula E-2a, A₁ to A₅ may each independently be N or C(Rᵢ). In Formula E-2a, Rₐ to Rᵢ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. For example, Rₐ to Rᵢ may be bonded to an adjacent group to form a hydrocarbon ring or a heterocycle including N, O, S, etc., as a ring-forming atom.

In Formula E-2a, two or three of A₁ to A₅ may be N, and the remainder of A₁ to A₅ may be C(Rᵢ).

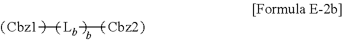

In Formula E-2b, Cbz1 and Cbz2 may each independently be an unsubstituted carbazole group or a carbazole group which is substituted with an aryl group having 6 to 30 ring-forming carbon atoms. In Formula E-2b, L_{b} may be a direct linkage, a substituted or unsubstituted arylene group having 6 to 30 ring-forming carbon atoms, or a substituted or unsubstituted heteroarylene group having 2 to 30 ring-forming carbon atoms. In Formula E-2b, b may be an integer from 0 to 10. When b is 2 or more, multiple L_{b} groups may each independently be a substituted or unsubstituted arylene group having 6 to 30 ring-forming carbon atoms, or a substituted or unsubstituted heteroarylene group having 2 to 30 ring-forming carbon atoms.

The compound represented by Formula E-2a or Formula E-2b may be any one selected from Compound Group E-2. However, the compounds listed in Compound Group E-2 are only examples, and the compound represented by Formula E-2a or Formula E-2b is not limited to Compound Group E-2.

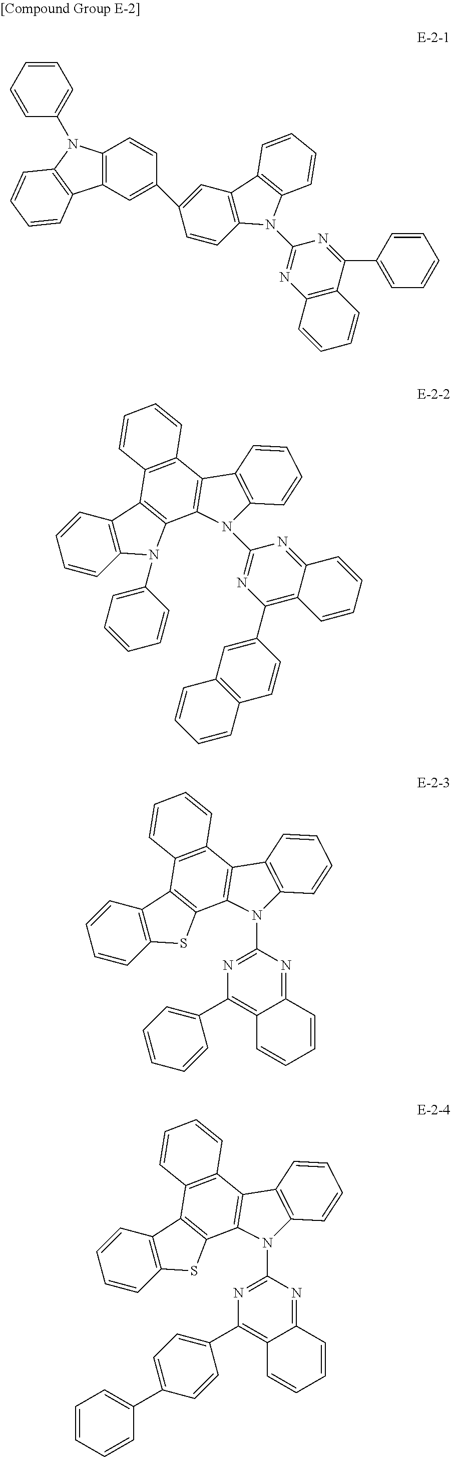 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-n2c3ccccc3c3c4ccccc4c4c5ccccc5n(-c5nc(-c6ccc7ccccc7c6)c6ccccc6n5)c4c32)cc1 ; c1ccc(-c2nc(-n3c4ccccc4c4c5ccccc5c5c6ccccc6sc5c43)nc3ccccc23)cc1 ; c1ccc(-c2ccc(-c3nc(-n4c5ccccc5c5c6ccccc6c6c7ccccc7sc6c54)nc4ccccc34)cc2)cc1]

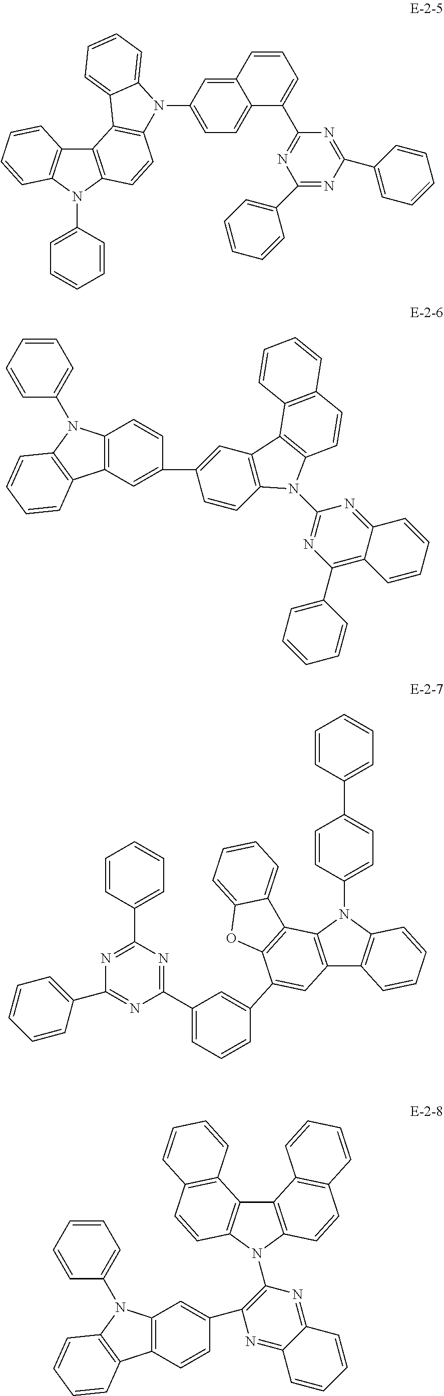 [SMILES: c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1]

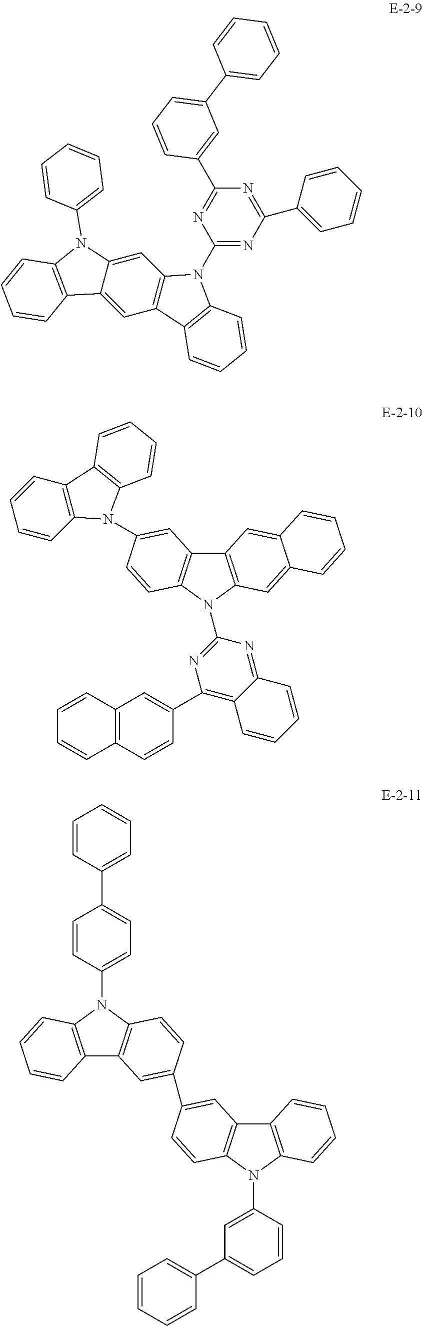 [SMILES: c1ccc(-c2cccc(-c3nc(-c4ccccc4)nc(-n4c5ccccc5c5cc6c7ccccc7n(-c7ccccc7)c6cc54)n3)c2)cc1 ; c1ccc2cc(-c3nc(-n4c5ccc(-n6c7ccccc7c7ccccc76)cc5c5cc6ccccc6cc54)nc4ccccc34)ccc2c1 ; c1ccc(-c2ccc(-n3c4ccccc4c4cc(-c5ccc6c(c5)c5ccccc5n6-c5cccc(-c6ccccc6)c5)ccc43)cc2)cc1]

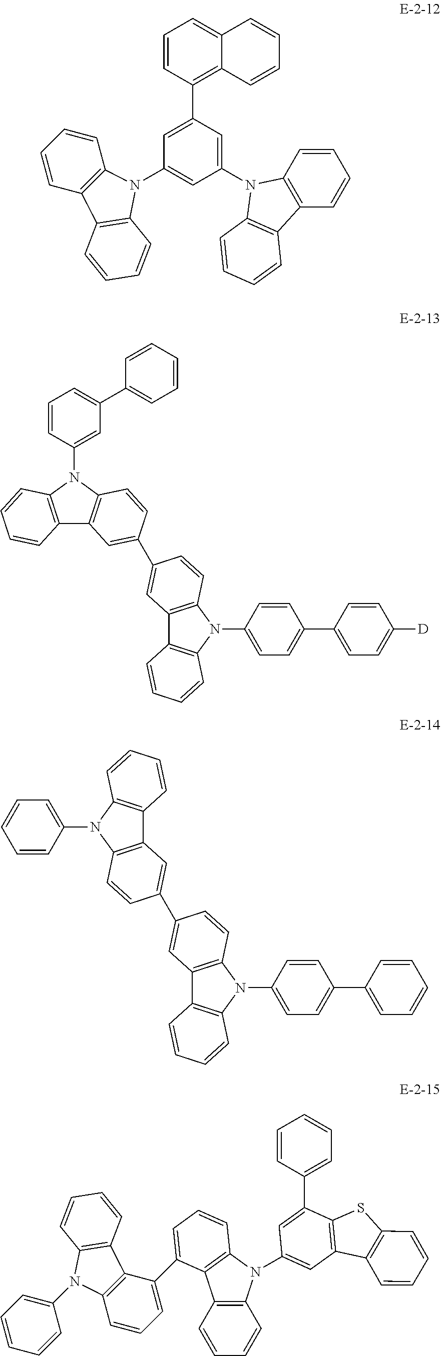 [SMILES: c1ccc2c(-c3cc(-n4c5ccccc5c5ccccc54)cc(-n4c5ccccc5c5ccccc54)c3)cccc2c1 ; [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

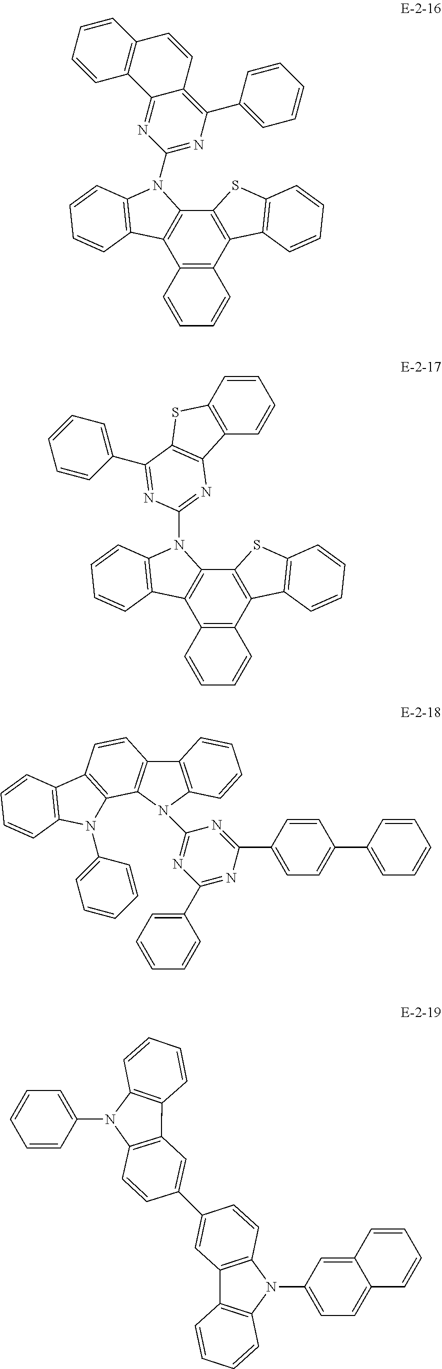 [SMILES: c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1 ; c1ccc(-c2ccc(-c3nc(-c4ccccc4)nc(-n4c5ccccc5c5ccc6c7ccccc7n(-c7ccccc7)c6c54)n3)cc2)cc1 ; c1ccc(-n2c3ccccc3c3cc(-c4ccc5c(c4)c4ccccc4n5-c4ccc5ccccc5c4)ccc32)cc1]

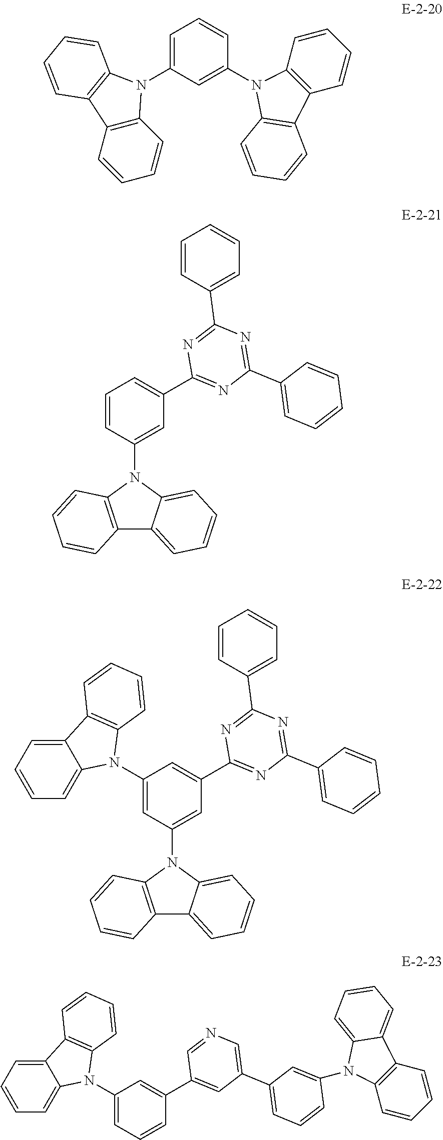 [SMILES: c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1]

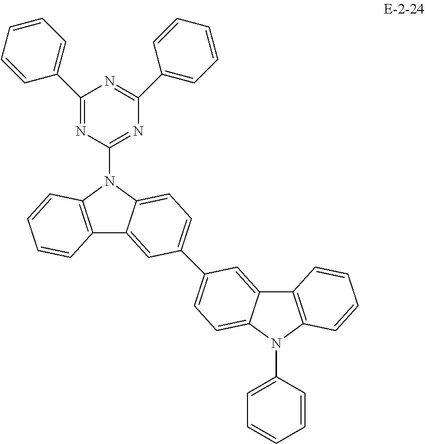 [SMILES: c1ccc(-c2nc(-c3ccccc3)nc(-n3c4ccccc4c4cc(-c5ccc6c(c5)c5ccccc5n6-c5ccccc5)ccc43)n2)cc1]

The light-emitting layer EML may further include a material of the related art as a host material. For example, the light-emitting layer EML may include as a host material, at least one of, bis (4-(9H-carbazol-9-yl) phenyl) diphenylsilane (BCPDS), (4-(1-(4-(diphenylamino) phenyl) cyclohexyl) phenyl) diphenyl-phosphine oxide (POPCPA), bis[2-(diphenylphosphino)phenyl] ether oxide (DPEPO), 4,4′-bis(N-carbazolyl)-1,1′-biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA) or 1,3,5-tris(1-phenyl-1H-benzo[d]imidazole-2-yl)benzene (TPBi). However, embodiments are not limited thereto. For example, tris(8-hydroxyquinolino)aluminum (Alq₃), 9,10-di(naphthalene-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenyl silyl)benzene (UGH2), hexaphenylcyclotrisiloxane (DPSiO₃), octaphenylcyclotetra siloxane (DPSiO₄), etc., may be used as the host material.

The light-emitting layer EML may include a compound represented by Formula M-a or Formula M-b. The compound represented by Formula M-a or Formula M-b may be used as a phosphorescence dopant material. In an embodiment, a compound represented by Formula M-a or Formula M-b may be used as an auxiliary dopant material.

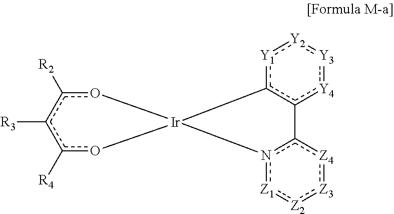

In Formula M-a, Y₁ to Y₄ and Z₁ to Z₄ may each independently be C(R₁) or N, and R₁ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. In Formula M-a, m may be 0 or 1, and n may be 2 or 3. In Formula M-a, when m is 0, n may be 3, and when m is 1, n may be 2.

The compound represented by Formula M-a may be used as a phosphorescence dopant material.

The compound represented by Formula M-a may be any one selected from Compounds M-a1 to M-a25. However, Compounds M-a1 to M-a25 are only examples, and the compound represented by Formula M-a is not limited to Compounds M-a1 to M-a25.

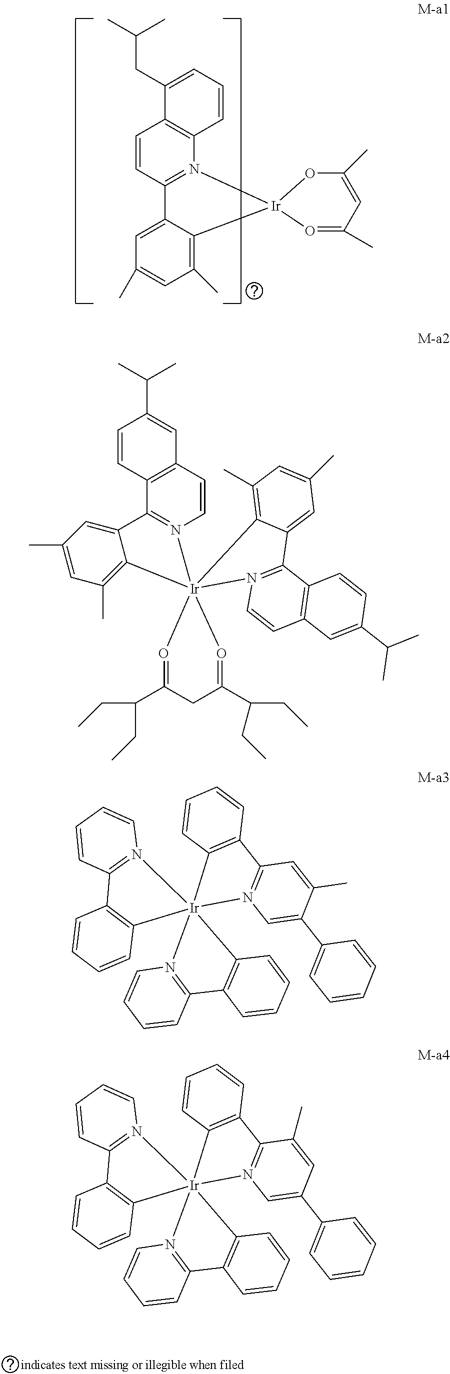

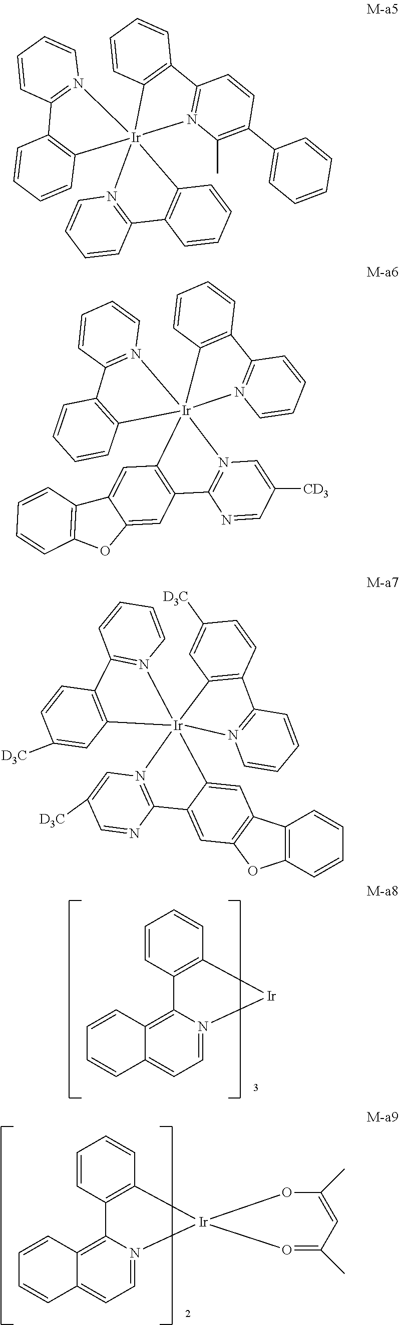

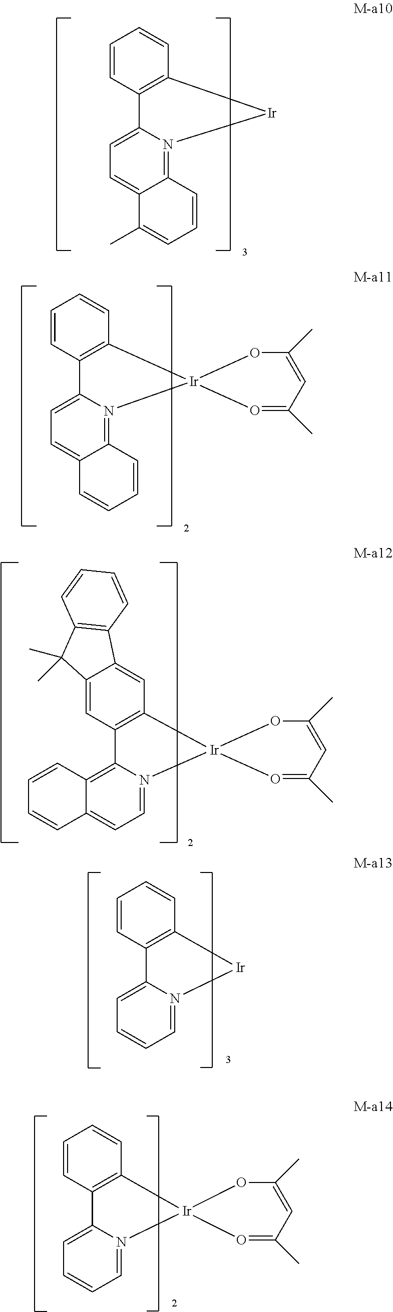

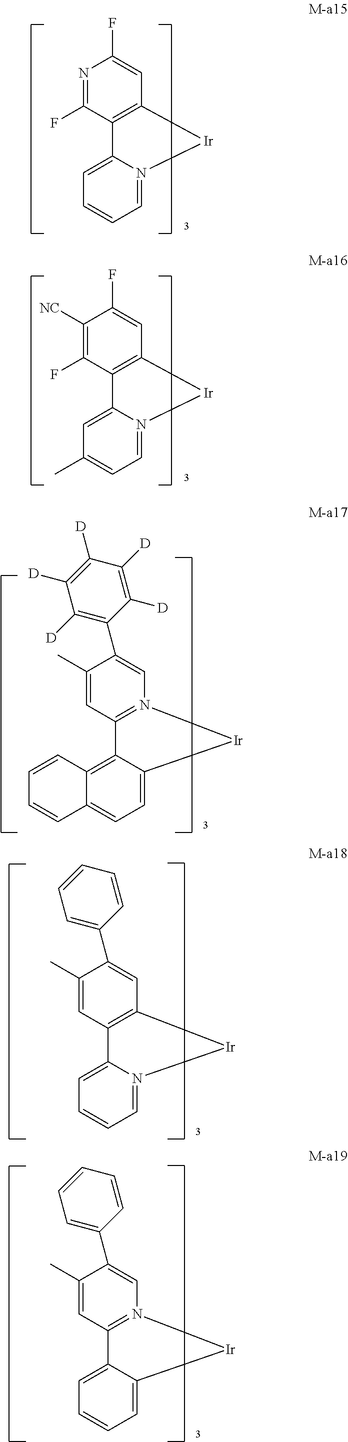

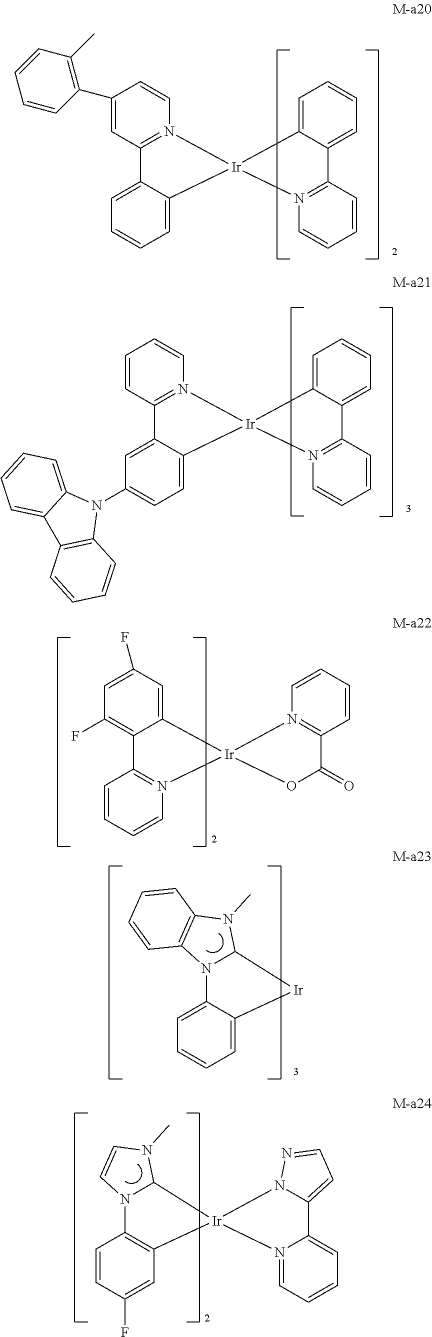

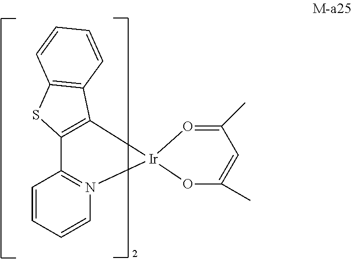

Compound M-a1 and Compound M-a2 may be used as red dopant materials, and Compound M-a3 to Compound M-a7 may be used as green dopant materials.

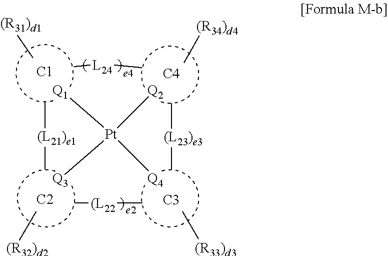

In Formula M-b, Q₁ to Q₄ may each independently be C or N, and C1 to C4 may each independently be a substituted or unsubstituted hydrocarbon ring having 5 to 30 ring-forming carbon atoms, or a substituted or unsubstituted heterocycle having 2 to 30 ring-forming carbon atoms. In Formula M-b, L₂₁ to L₂₄ may each independently be a direct linkage,

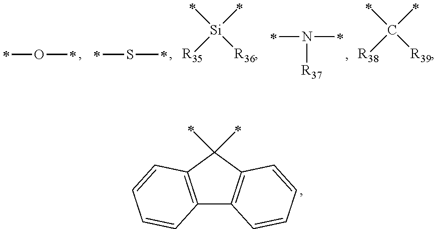

a substituted or unsubstituted divalent alkyl group having 1 to 20 carbon atoms, a substituted or unsubstituted arylene group having 6 to 30 ring-forming carbon atoms, or a substituted or unsubstituted heteroarylene group having 2 to 30 ring-forming carbon atoms, and e1 to e4 may each independently be 0 or 1. In Formula M-b, R₃₁ to R₃₉ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring, and d1 to d4 may each independently be an integer from 0 to 4.

The compound represented by Formula M-b may be used as a blue phosphorescence dopant or a green phosphorescence dopant. In an embodiment, the compound represented by Formula M-b may be further included in the light-emitting layer EML as an auxiliary dopant.

The compound represented by Formula M-b may be any one selected from Compounds M-b-1 to M-b-11. However, Compounds M-b-1 to M-b-11 are only examples, and the compound represented by Formula M-b is not limited to Compounds M-b-1 to M-b-11.

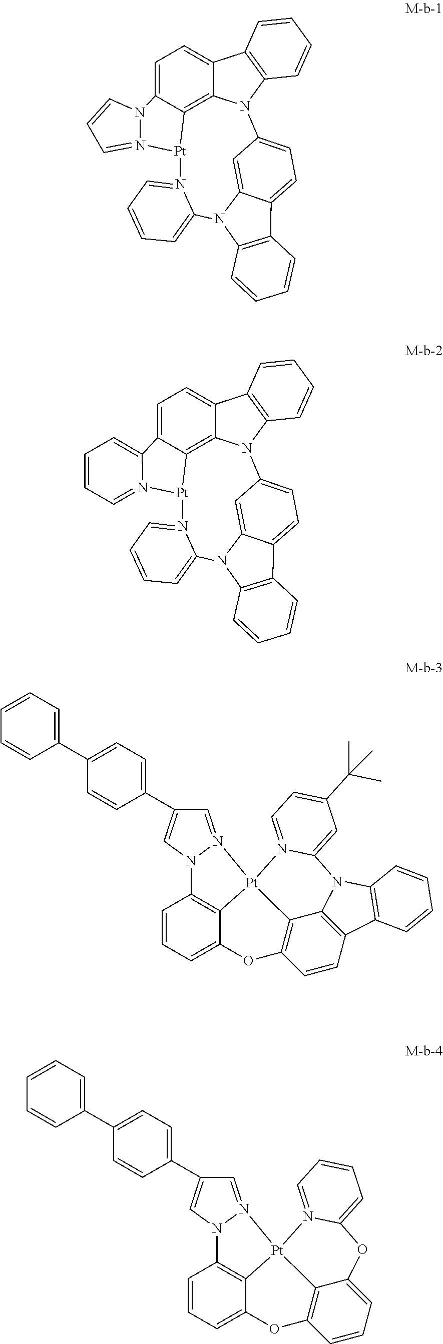

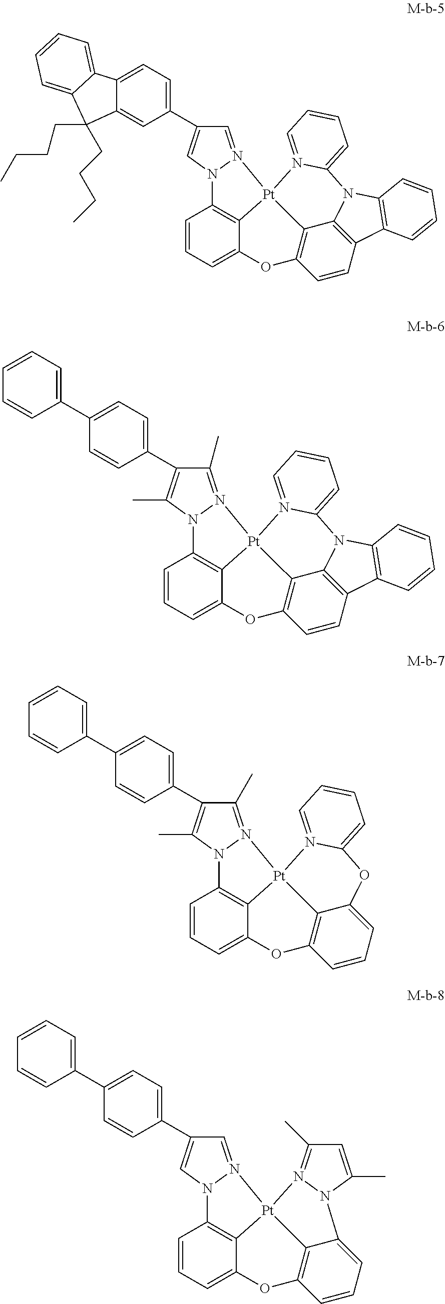

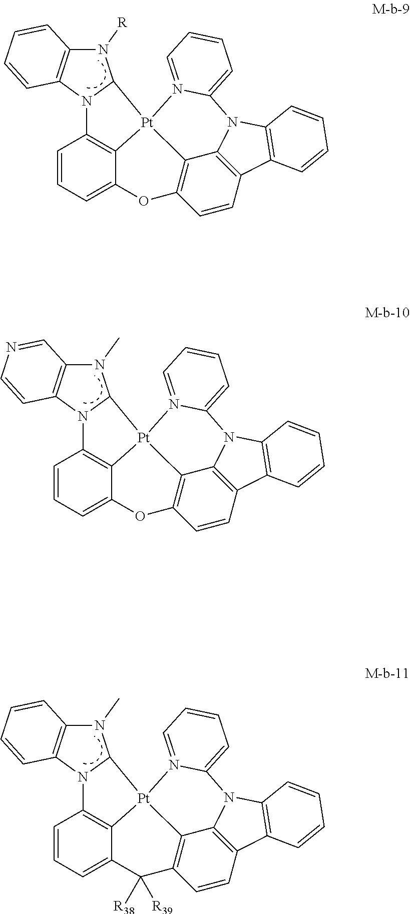

In Compounds M-b-1 to M-b-11, R, R₃₈, and R₃₉ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

The light-emitting layer EML may include a compound represented by any one of Formulas F-a to F-c. The compounds represented by Formula F-a to Formula F-c may be used as fluorescence dopant materials.

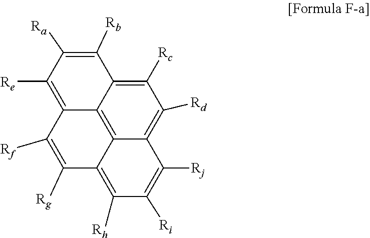

In Formula F-a, two of Rₐ to Rⱼ may each independently be substituted with a group represented by *—NAr₁Ar₂. The remainder of Rₐ to Rⱼ which are not substituted with the group represented by *—NAr₁Ar₂ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In the group represented by *—NAr₁Ar₂, Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. For example, at least one of Ar₁ and Ar₂ may be a heteroaryl group including O or S as a ring-forming atom.

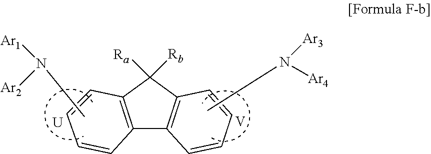

In Formula F-b, Rₐ and R_{b} may each independently be a hydrogen atom, a deuterium atom, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. In Formula F-b, Ar₁ to Ar₄ may each independently be a substituted or unsubstituted aryl group having 6 to 30 ring-forming carbon atoms, or a substituted or unsubstituted heteroaryl group having 2 to 30 ring-forming carbon atoms.

In Formula F-b, U and V may each independently be a substituted or unsubstituted hydrocarbon ring of 5 to 30 ring-forming carbon atoms, or a substituted or unsubstituted heterocycle having 2 to 30 ring-forming carbon atoms.

In Formula F-b, the number of rings represented by U and V may each independently be 0 or 1. For example, in Formula F-b, when the number of U or V is 1, a fused ring may be present at a part designated as U or V, and when the number of U or V is 0, a fused ring may be present at the part designated as U or V. When the number of U is 0 and the number of V is 1, or when the number of U is 1 and the number of V is 0, a fused ring having a fluorene core of Formula F-b may be a cyclic compound with four rings. When both the number of U and the number of V are 0, the fused ring having a fluorene core of Formula F-b may be a cyclic compound with three rings. When both the number of U and the number of V are 1, a fused ring having a fluorene core of Formula F-b may be a cyclic compound with five rings.

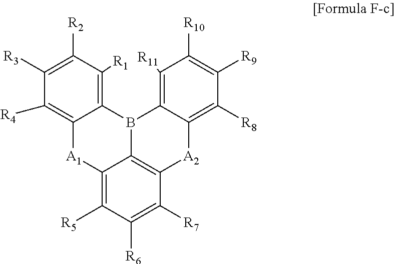

In Formula F-c, A₁ and A₂ may each independently be O, S, Se, or N(Rₘ), and Rₘ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In Formula F-c, R₁ to R₁₁ may each independently be a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring.

In Formula F-c, A₁ and A₂ may each independently be bonded to substituents of an adjacent ring to form a fused ring. For example, when A₁ and A₂ are each independently N(Rₘ), A₁ may be bonded to R₄ or R₅ to form a ring. For example, A₂ may be bonded to R₇ or R₈ to form a ring.

In an embodiment, the light-emitting layer EML may include, a dopant material of the related art, such as styryl derivatives (for example, 1,4-bis[2-(3-N-ethylcarbazoryl)vinyl]benzene (BCzVB), 4-(di-p-tolylamino)-4′-[(di-p-tolylamino)styryl] stilbene (DPAVB), N-(4-((E)-2-(6-((E)-4-(diphenylamino)styryl)naphthalen-2-yl)vinyl)phenyl)-N-phenylbenzenamine(N-BDAVBi)), 4,4′-bis[2-(4-(N,N-diphenylamino)phenyl)vinyl]biphenyl (DPAVBi), perylene and the derivatives thereof (for example, 2, 5, 8, 11-Tetra-t-butylperylene(TBP)), pyrene and the derivatives thereof (for example, 1, 1-dipyrene, 1, 4-dipyrenylbenzene, 1, 4-Bis(N, N-Diphenylamino)pyrene), etc.

In an embodiment, when multiple light-emitting layers EML are included, at least one light-emitting layer EML may include a phosphorescent dopant material of the related art. For example, the phosphorescence dopant material may include a metal complex including iridium (Ir), platinum (Pt), osmium (Os), gold (Au), titanium (Ti), zirconium (Zr), hafnium (Hf), europium (Eu), terbium (Tb) or thulium (Tm). For example, iridium(III) bis(4,6-difluorophenylpyridinato-N, C2′) (FIrpic), bis(2,4-difluorophenylpyridinato)-tetrakis(1-pyrazolyl)borate iridium(III) (Fir6), or platinum octaethyl porphyrin (PtOEP) may be used as the phosphorescence dopant material. However, embodiments are not limited thereto.

In an embodiment, the light-emitting layer EML may include a hole-transporting host and an electron-transporting host. The light-emitting layer EML may include an auxiliary dopant and a light-emitting dopant. The auxiliary dopant may include a phosphorescent dopant material or a thermally activated delayed fluorescence dopant material. For example, in an embodiment, the light-emitting layer EML may include a hole transport host, an electron transport host, an auxiliary dopant, and a light-emitting dopant.

In the light-emitting layer EML, an exciplex may be formed by a hole-transporting host and an electron-transporting host. A triplet energy of the exciplex formed by a hole-transporting host and an electron-transporting host may correspond to an interval T1 between a lowest unoccupied molecular orbital (LUMO) energy level of the electron-transporting host and a highest occupied molecular orbital (HOMO) energy level of the hole-transporting host.

In an embodiment, the triplet energy (T1) of the exciplex formed by the hole-transporting host and the electron-transporting host may be in a range of about 2.4 eV to about 3.0 eV. The triplet energy level of the exciplex may be a value smaller than an energy gap of each host material. For example, the exciplex may have a triplet energy level equal to or less than about 3.0 eV, which may be an energy gap between the hole-transporting host and the electron-transporting host.

At least one light-emitting layer EML may include a quantum dot material. The quantum dot may be selected from a Group II-VI compound, a Group III-VI compound, a Group compound, a Group III-V compound, a Group III-II-V compound, a Group IV-VI compound, a Group IV element, a Group IV compound, and combinations thereof.

The Group II-VI compound may be selected from: a binary compound selected from the group consisting of CdSe, CdTe, CdS, ZnS, ZnSe, ZnTe, ZnO, HgS, HgSe, HgTe, MgSe, MgS, and mixtures thereof; a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and mixtures thereof; a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, HgZnSTe, and mixtures thereof; or any combination thereof.

The Group III-VI compound may be selected from: a binary compound such as In₂S₃, and In₂Se₃; a ternary compound such as InGaS₃, and InGaSe₃; or any combination thereof.

The Group compound may be selected from: a ternary compound selected from the group consisting of AgInS, AgInS₂, CuInS, CuInS₂, AgGaS₂, CuGaS₂ CuGaO₂, AgGaO₂, AgAlO₂ and mixtures thereof; a quaternary compound such as AgInGaS₂, and CuInGaS₂, and the like; or any combination thereof.

The Group III-V compound may be selected from: a binary compound selected from the group consisting of GaN, GaP, GaAs, GaSb, AlN, AlP, AlAs, AlSb, InN, InP, InAs, InSb, and mixtures thereof; a ternary compound selected from the group consisting of GaNP, GaNAs, GaNSb, GaPAs, GaPSb, AlNP, AINAs, AlNSb, AlPAs, AlPSb, InGaP, InAlP, InNP, InNAs, InNSb, InPAs, InPSb, and mixtures thereof; a quaternary compound selected from the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and mixtures thereof; or any combination thereof. The Group III-V compound may further include a Group II metal. For example, InZnP, etc., may be selected as a Group III-II-V compound.

The Group IV-VI compound may be selected from: a binary compound selected from the group consisting of SnS, SnSe, SnTe, PbS, PbSe, PbTe, and mixtures thereof; a ternary compound selected from the group consisting of SnSeS, SnSeTe, SnSTe, PbSeS, PbSeTe, PbSTe, SnPbS, SnPbSe, SnPbTe, and mixtures thereof; a quaternary compound selected from the group consisting of SnPbSSe, SnPbSeTe, SnPbSTe, and mixtures thereof; or any combination thereof. The Group IV element may be selected from the group consisting of Si, Ge, and a mixture thereof. The Group IV compound may be a binary compound selected from the group consisting of SiC, SiGe, and a mixture thereof.

A binary compound, a ternary compound, or a quaternary compound may be present in a particle at a uniform concentration, or may be present in a particle at partially different concentrations. In an embodiment, a quantum dot may have a core/shell structure in which a quantum dot surrounds another quantum dot. A quantum dot having a core/shell structure may have a concentration gradient in which the concentration of an element that is present in the shell decreases toward the center thereof.

In embodiments, the quantum dot may have a core-shell structure including a core having the above-described nanocrystals and a shell surrounding the core. The shell of the quantum dot may serve as a protective layer for maintaining semiconductor characteristics by preventing chemical modification of the core and/or may serve as a charging layer for imparting electrophoretic properties to the quantum dot. The shell may be a single layer or a multilayer. Examples of the shell of the quantum dot may include a metal oxide, a non-metal oxide, a semiconductor compound, or combinations thereof.

For example, examples of the metal oxide or the non-metal oxide may include a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄ and NiO, or a ternary compound such as MgAl₂O₄, CoFe₂O₄, NiFe₂O₄ and CoMn₂O₄, but embodiments are not limited thereto.

Examples of the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but embodiments are not limited thereto.

The quantum dot may have a full width of half maximum (FWHM) of an emission wavelength spectrum equal to or less than about 45 nm. For example, the quantum dot may have a FWHM of an emission wavelength spectrum equal to or less than about 40 nm. For example, the quantum dot may have a FWHM of an emission wavelength spectrum equal to or less than about 30 nm. Within these ranges, color purity or color reproducibility may be improved. Light emitted through a quantum dot may be emitted in all directions, so that wide viewing angle characteristics may be improved.

The shape of the quantum dot may be any form that is used in the related art, without limitation. For example, the quantum dot may have a spherical shape, a pyramidal shape, a multi-arm shape, or a cubic shape, or the quantum dot may be in the form of a nanoparticle, a nanotube, a nanowire, a nanofiber, a nanoplatelet, etc.

The quantum dot may control the color of emitted light according to a particle size thereof, and accordingly, the quantum dot may have various emission colors such as blue, red, or green.

In the light-emitting element ED according to an embodiment, as illustrated in FIGS. 3 to 7, the electron transport region ETR is provided on the light-emitting layer EML. The electron transport region ETR may include at least one of a hole blocking layer HBL, an electron transport layer ETL, or an electron injection layer EIL. However, embodiments are not limited thereto.

The electron transport region ETR may be a layer formed of a single material, a layer formed of different materials, or a structure having multiple layers formed of different materials.

For example, the electron transport region ETR may have a structure of single layer of an electron injection layer EIL or an electron transport layer ETL, or may have a single layer structure formed of an electron injection material and an electron transport material. In other embodiments, the electron transport region ETR may have a structure of single layer formed of different materials, or may have a structure in which an electron transport layer ETL/electron injection layer EIL, a hole blocking layer HBL/electron transport layer ETL/electron injection layer EIL, or an electron transport layer ETL/buffer layer (not shown)/electron injection layer EIL are stacked in its respective stated order from the light-emitting layer EML, but embodiments are not limited thereto. The electron transport region ETR may have a thickness, for example, in a range of about 1,000 Å to about 1,500 Å.

The electron transport region ETR may be formed of various methods such as a vacuum deposition method, a spin coating method, a cast method, a Langmuir-Blodgett (LB) method, an inkjet printing method, a laser printing method, and a laser induced thermal imaging (LITI) method.

The electron transport region ETR may include a compound represented by Formula ET-1.

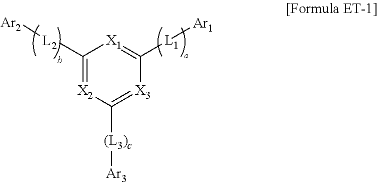

In Formula ET-1, at least one of X₁ to X₃ may be N, and the remainder of X₁ to X₃ may be C(Rₐ); and Rₐ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In Formula ET-1, Ar₁ to Ar₃ may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In Formula ET-1, a to c may each independently be an integer from 0 to 10. In Formula ET-1, L₁ to L₃ may each independently be a direct linkage, a substituted or unsubstituted arylene group having 6 to 30 ring-forming carbon atoms, or a substituted or unsubstituted heteroarylene group having 2 to 30 ring-forming carbon atoms. When a to c are 2 or more, multiple groups of L₁ to L₃ may each independently be a substituted or unsubstituted arylene group having 6 to 30 ring-forming carbon atoms, or a substituted or unsubstituted heteroarylene group having 2 to 30 ring-forming carbon atoms.

The electron transport region ETR may include an anthracene-based compound. However, embodiments are not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazol-1-yl)phenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-Biphenylyl)-5-(4-tert-butylphenyl)-1,3,4-oxadiazole (tBu-PBD), bis(2-methyl-8-quinolinolato-N1,O8)-(1,1′-Biphenyl-4-olato)aluminum (BAlq), berylliumbis(benzoquinolin-10-olate) (Bebq₂), 9,10-di(naphthalene-2-yl)anthracene (ADN), 1,3-bis[3,5-di(pyridin-3-yl)phenyl]benzene (BmPyPhB), diphenyl(4-(triphenylsilyl)phenyl)phosphine oxide (TSPO1), and mixtures thereof.

The electron transport region ETR may include at least one of Compounds ET1 to ET36.

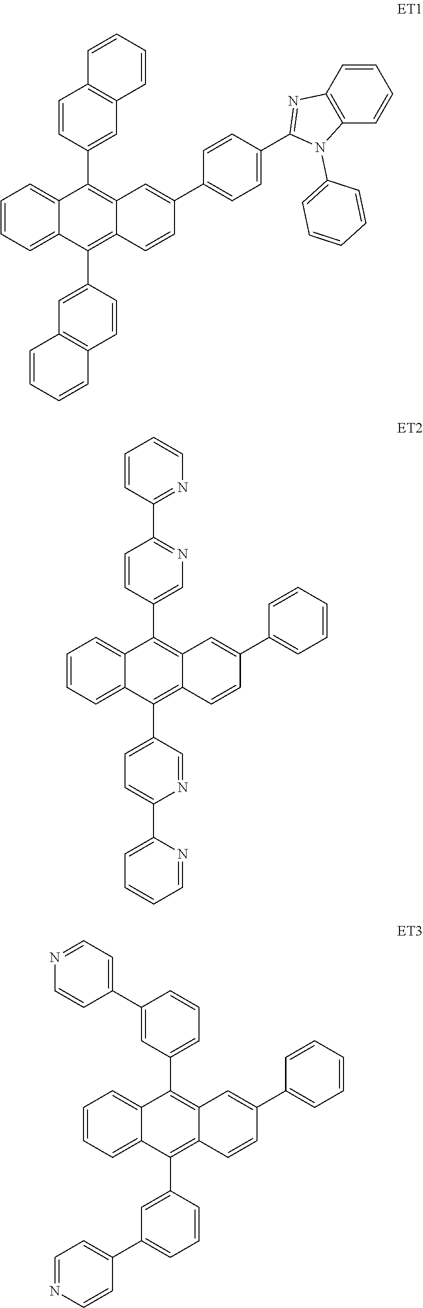 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

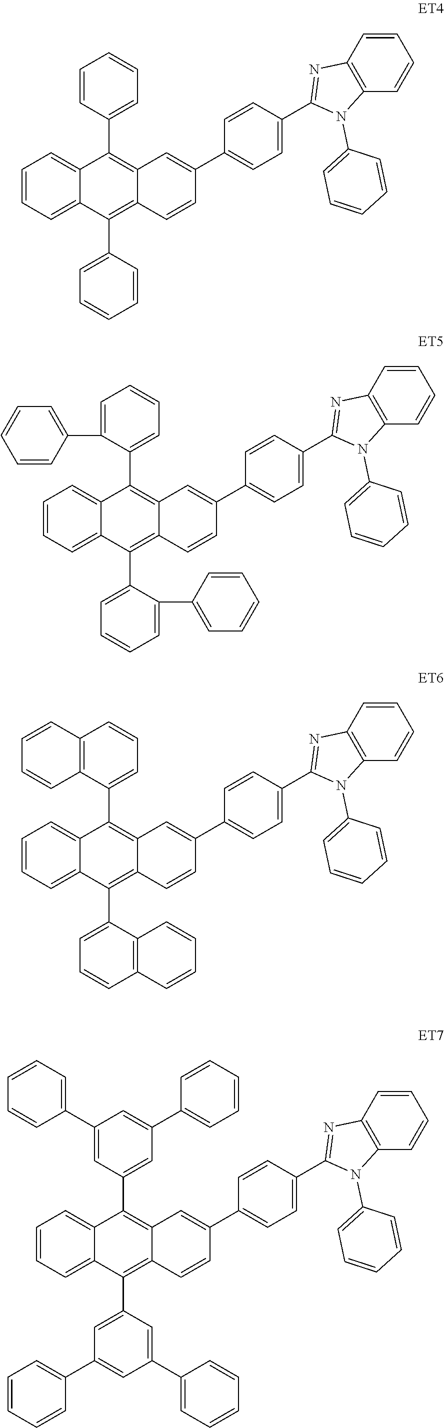 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

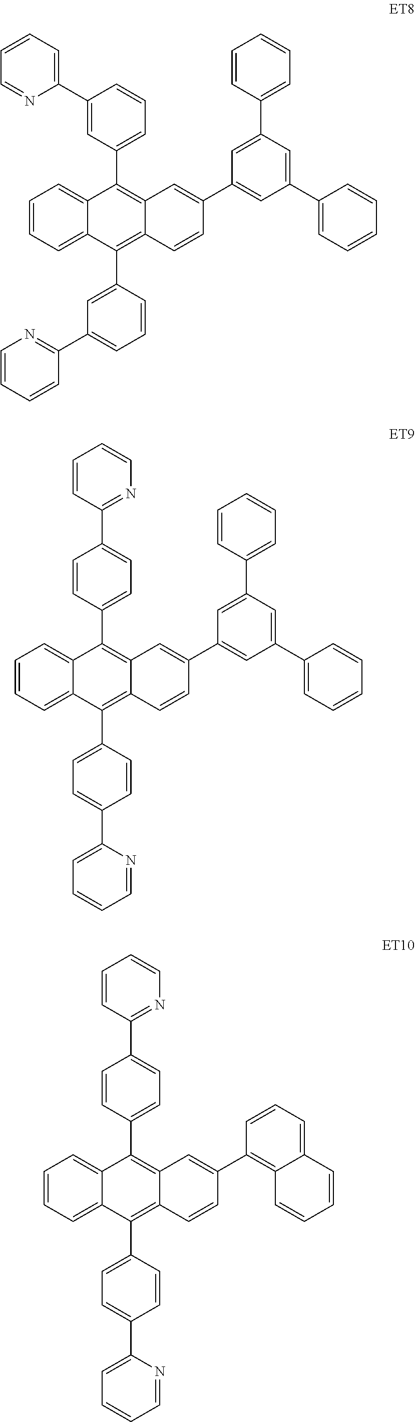 [SMILES: c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1 ; c1ccc(-c2ccc(-c3c4ccccc4c(-c4ccc(-c5ccccn5)cc4)c4cc(-c5cccc6ccccc56)ccc34)cc2)nc1]

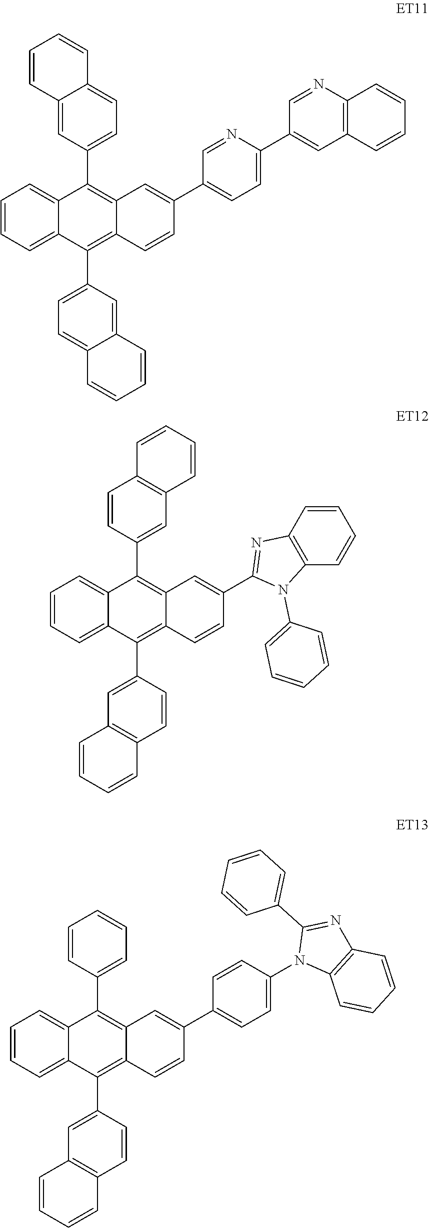

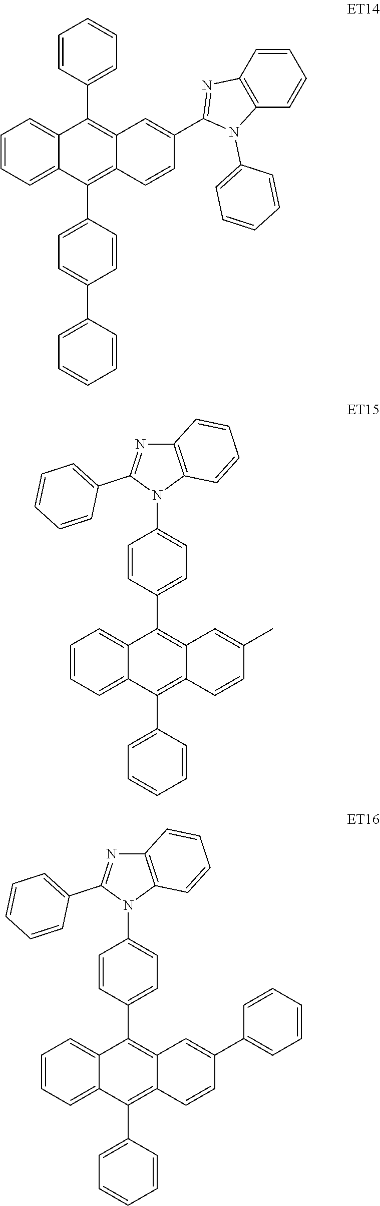

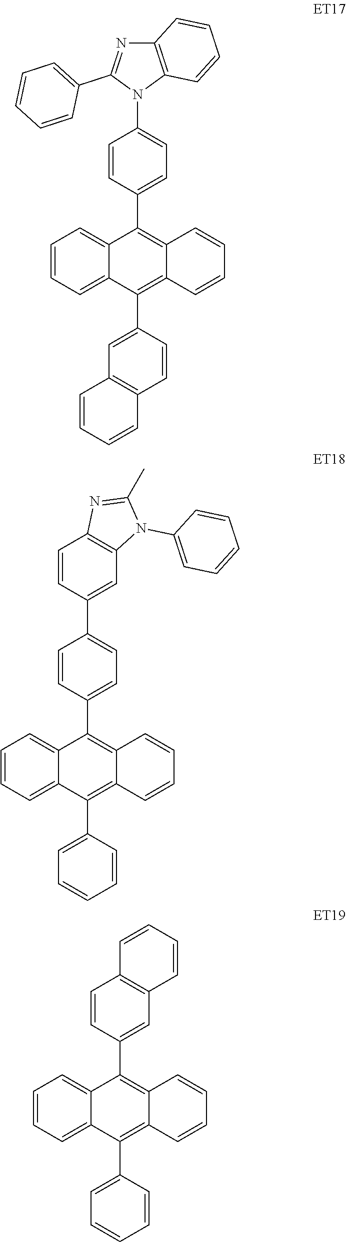

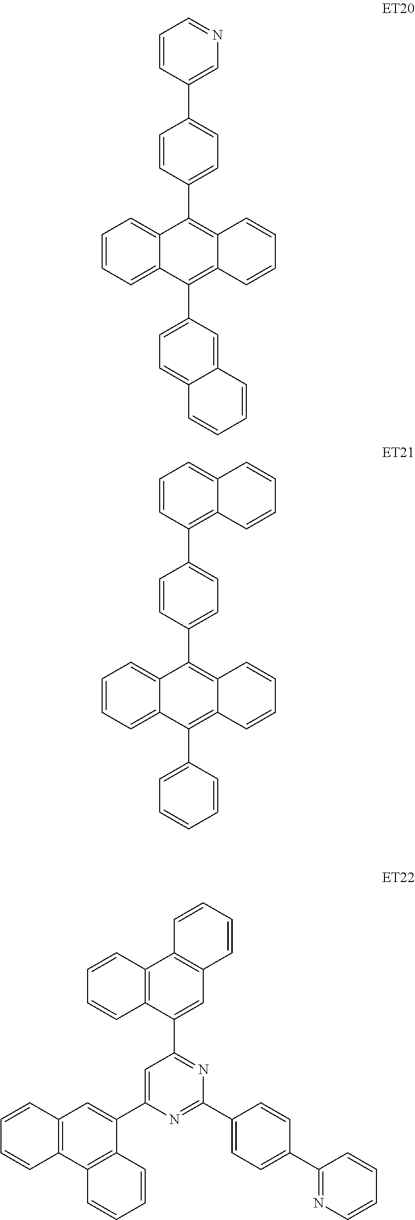

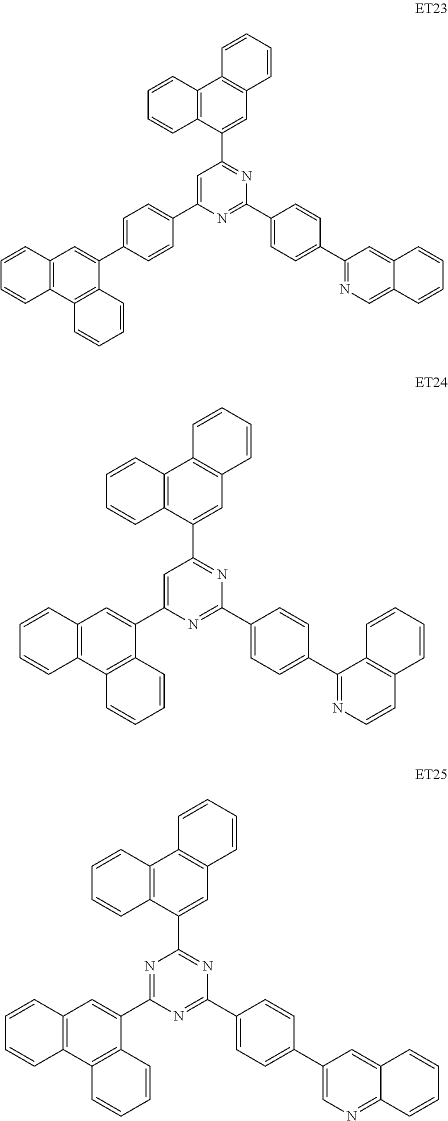 [SMILES: c1ccc2cc(-c3ccc(-c4nc(-c5ccc(-c6cc7ccccc7c7ccccc67)cc5)cc(-c5cc6ccccc6c6ccccc56)n4)cc3)ncc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1]

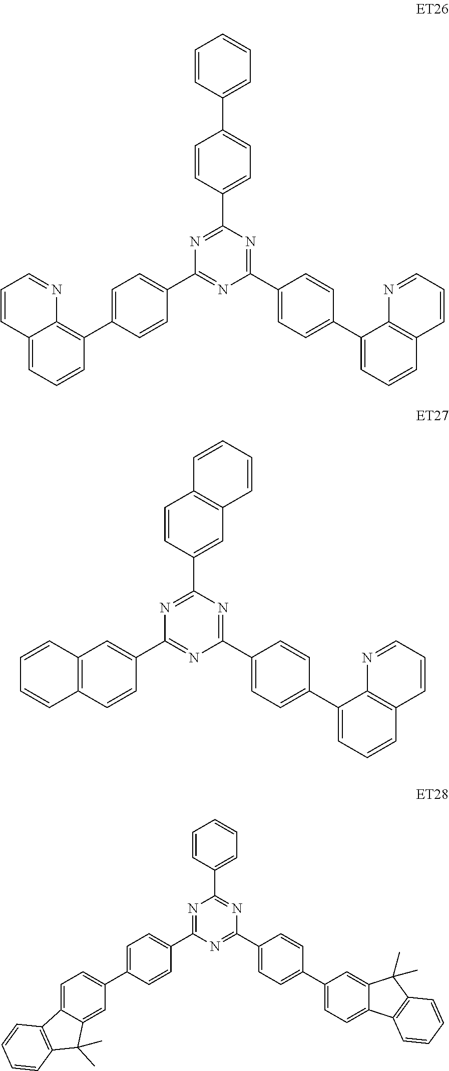 [SMILES: c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21]

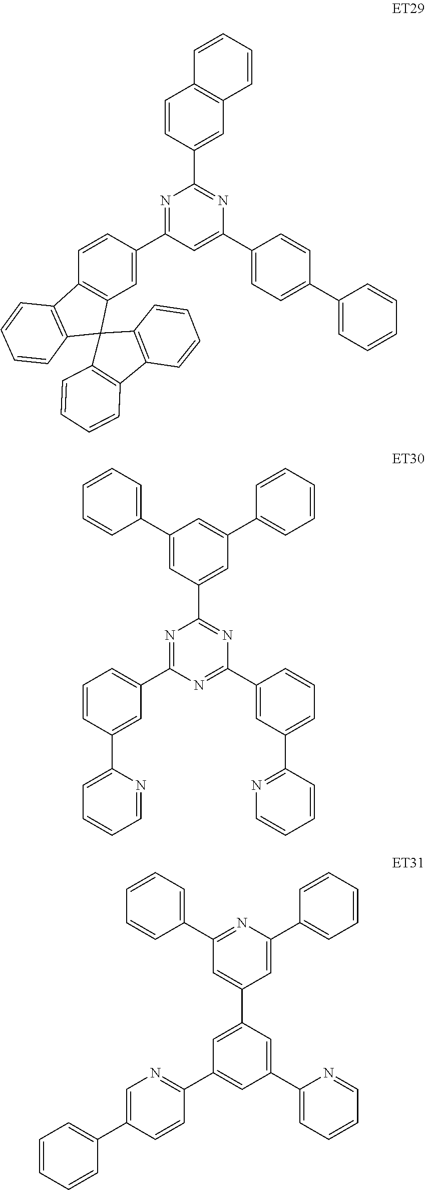

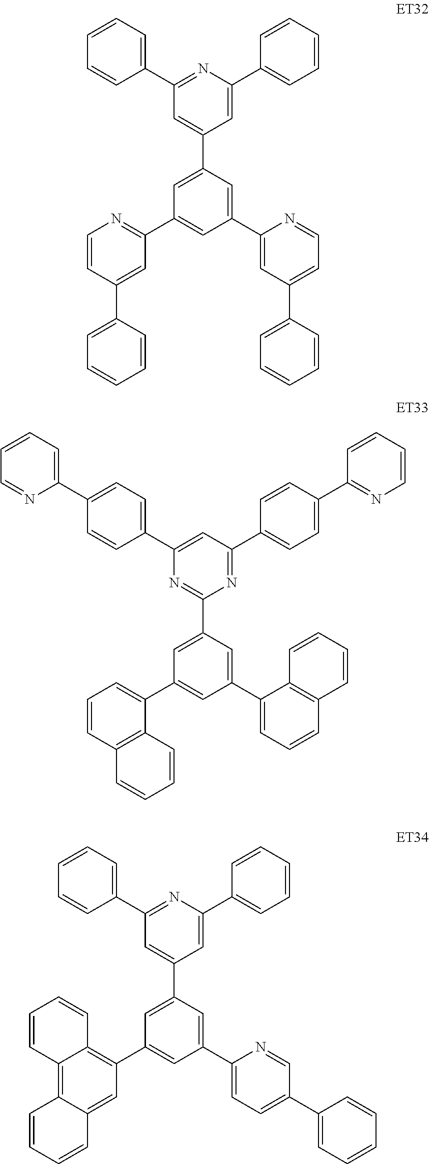 [SMILES: c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1]

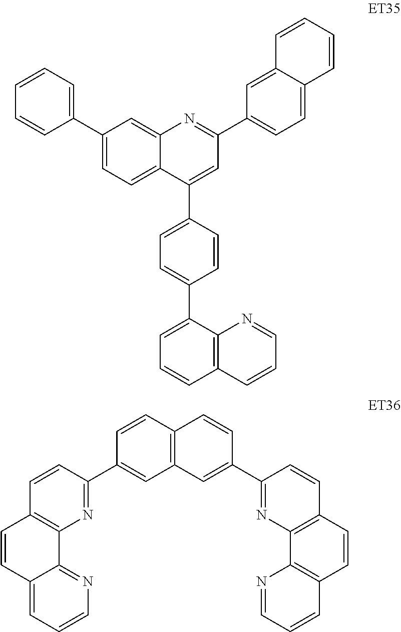

The electron transport region ETR may include a metal halide such as LiF, NaCl, CsF, RbCl, RbI, CuI, or KI, a lanthanide metal such as Yb, or a co-deposition material of a metal halide and a lanthanide metal. For example, the electron transport region ETR may include KI:Yb, RbI:Yb, and the like as the co-deposition material. The electron transport region ETR may include a metal oxide such as Li₂O and BaO, or 8-hydroxy-lithium quinolate (Liq), but embodiments are not limited thereto. The electron transport region ETR may also be formed of a mixture material of an electron transport material and an insulating organometallic salt. The organometallic salt may be a material having an energy band gap equal to or greater than about 4 eV. For example, the organometallic salt may include metal acetates, metal benzoates, metal acetoacetates, metal acetylacetonates, or metal stearates.

The electron transport region ETR may further include at least one of 2,9-dimethyl-4,7-diphenyl-1,10-phenanthroline (BCP), diphenyl(4-(triphenylsilyl)phenyl)phosphine oxide (TSPO1) or 4,7-diphenyl-1,10-phenanthroline (Bphen) in addition to the above-described materials. However, embodiments are not limited thereto.

The electron transport region ETR may include the above-described compounds of the electron transport region ETR in at least one of an electron injection layer EIL, an electron transport layer ETL, or a hole blocking layer HBL.

When the electron transport region ETR includes an electron transport layer ETL, the electron transport layer ETL may have a thickness in a range of about 100 Å to about 1,000 Å. For example, the electron transport layer ETL may have a thickness in a range of about 150 Å to about 500 Å. When the thickness of the electron transport layer ETL satisfies the above-described range, satisfactory electron transport properties may be obtained without a substantial increase in driving voltage. When the electron transport region ETR includes an electron injection layer EIL, the electron injection layer EIL may have a thickness in a range of about 1 Å to about 100 Å. For example, the electron injection layer EIL may have a thickness in a range of about 3 Å to about 90 Å. When the thickness of the electron injection layer EIL satisfies the above-described range, satisfactory electron injection properties may be obtained without a substantial increase in driving voltage.

The second electrode EL2 is provided on the electron transport region ETR. The second electrode EL2 may be a common electrode. The second electrode EL2 may be a cathode or an anode, but embodiments are not limited thereto. For example, when the first electrode EL1 is an anode, the second cathode EL2 may be a cathode; and when the first electrode EL1 is a cathode, the second electrode EL2 may be an anode. The second electrode may include at least one of Ag, Mg, Cu, Al, Pt, Pd, Au, Ni, Nd, Ir, Cr, Li, Ca, LiF, Mo, Ti, W, In, Sn, Zn, an oxide thereof, a compound thereof, or a mixture thereof.

The second electrode EL2 may be a transmissive electrode, a transflective electrode, or a reflective electrode. When the second electrode EL2 is a transmissive electrode, the second electrode EL2 may include a transparent metal oxide, for example, indium tin oxide (ITO), indium zinc oxide (IZO), zinc oxide (ZnO), indium tin zinc oxide (ITZO), etc.

When the second electrode EL2 is a transflective electrode or a reflective electrode, the second electrode EL2 may include Ag, Mg, Cu, Al, Pt, Pd, Au, Ni, Nd, Ir, Cr, Li, Ca, LiF/Ca (stacked structure of LiF and Ca), LiF/Al (stacked structure of LiF and Al), Mo, Ti, Yb, W, a compound thereof, or a mixture thereof (for example, AgMg, AgYb, or MgAg). In another embodiment, the second electrode EL2 may have a multilayered structure including a reflective film or a transflective film formed of the above-described materials and a transmissive conductive film formed of indium tin oxide (ITO), indium zinc oxide (IZO), zinc oxide (ZnO), indium tin zinc oxide (ITZO), etc. For example, the second electrode EL2 may include the above-described metal materials, combinations of two or more metal materials selected from the above-described metal materials, or oxides of the above-described metal materials.

Although not shown in the drawings, the second electrode EL2 may be electrically connected to an auxiliary electrode. When the second electrode EL2 is electrically connected to the auxiliary electrode, the resistance of the second electrode EL2 may be reduced.

In an embodiment, the light-emitting element ED may further include a capping layer CPL disposed on the second electrode EL2. The capping layer CPL may be a multilayer or a single layer.

In an embodiment, the capping layer CPL may include an organic layer or an inorganic layer. For example, when the capping layer CPL includes an inorganic material, the inorganic material may include an alkali metal compound such as LiF, an alkaline earth metal compound such as MgF₂, and SiON, SiNₓ, SiOy, etc.

For example, when the capping layer CPL includes an organic material, the organic material may include α-NPD, NPB, TPD, m-MTDATA, Alq₃, CuPc, N4,N4,N4′,N4′-tetra (biphenyl-4-yl) biphenyl-4,4′-diamine (TPD15), 4,4′,4″-tris (carbazol sol-9-yl) triphenylamine (TCTA), etc., or may include an epoxy resin, or acrylate such as methacrylate. However, embodiments are not limited thereto, and a capping layer CPL may include at least one of Compounds P1 to P5:

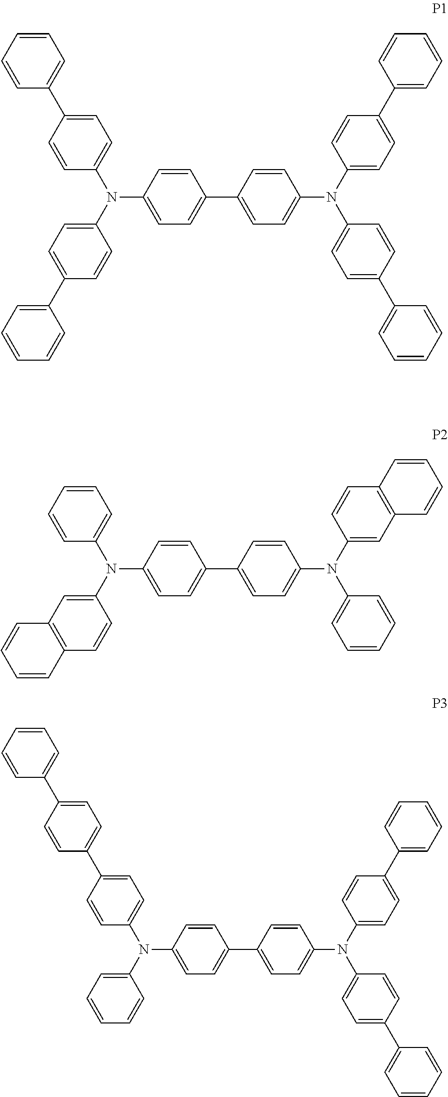 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

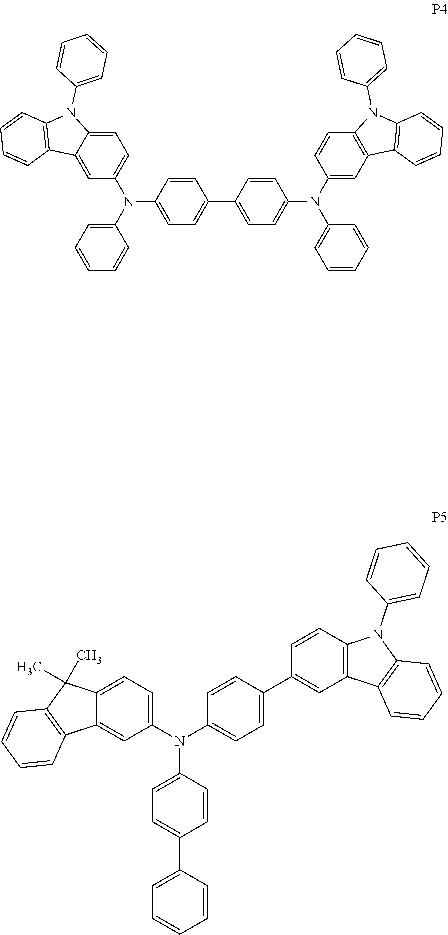 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2cc(N(c3ccc(-c4ccccc4)cc3)c3ccc(-c4ccc5c(c4)c4ccccc4n5-c4ccccc4)cc3)ccc21]

A refractive index of the capping layer CPL may be equal to or greater than about 1.6. For example, the refractive index of the capping layer CPL may be equal to or greater than about 1.6 with respect to light in a wavelength range of about 550 nm to about 660 nm.

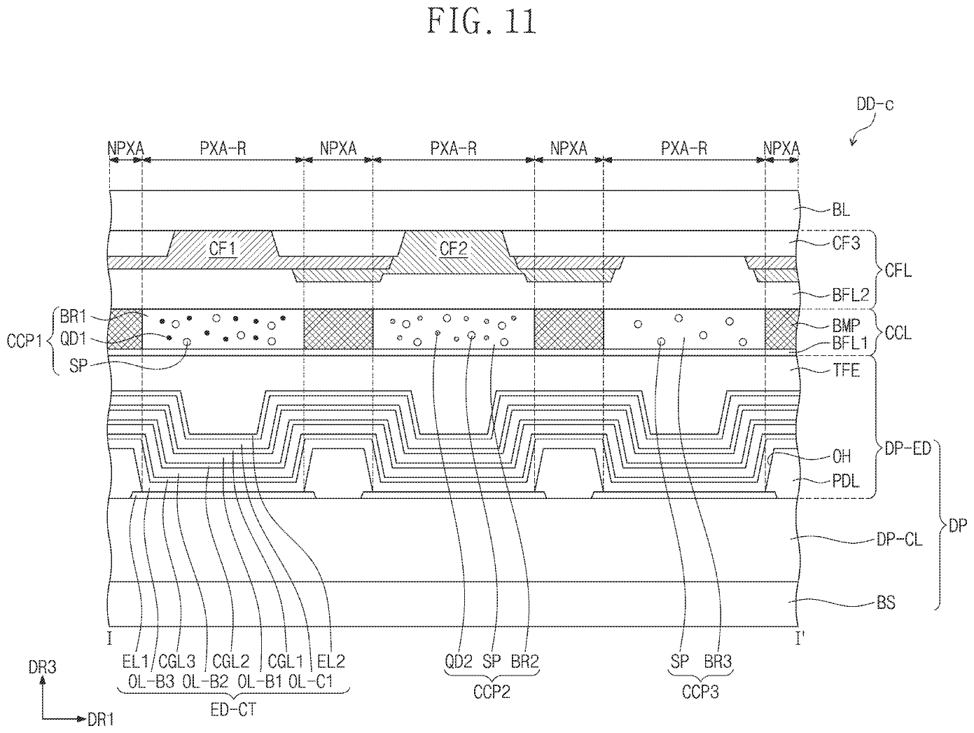

FIGS. 8 to 11 are each a schematic cross-sectional view of a display device according to embodiments. In the description of the display devices according to embodiments with reference to FIGS. 8 to 11, the features that have been described with reference to FIGS. 1 to 7 will not be described again, and the description will be focused on the differing features.

Referring to FIG. 8, the display device DD-a according to an embodiment may include a display panel DP including a display element layer DP-ED, a light control layer CCL disposed on the display panel DP, and a color filter layer CFL.

In an embodiment illustrated in FIG. 8, the display panel DP may include a base layer BS, a circuit layer DP-CL provided on the base layer BS, and a display element layer DP-ED, and the display element layer DP-ED may include a light-emitting element ED.

The light-emitting element ED may include a first electrode EL1, a hole transport region HTR disposed on the first electrode EL1, a light-emitting layer EML disposed on the hole transport region HTR, an electron transport region ETR disposed on the light-emitting layer EML, and a second electrode EL2 disposed on the electron transport region ETR. A structure of the light-emitting element ED illustrated in FIG. 8 may be the same as a structure of a light-emitting element according to one of FIGS. 3 to 7.

The hole transport region HTR of the light-emitting element ED included in the display device DD-a according to an embodiment may include the above-described amine compound according to an embodiment.

Referring to FIG. 8, the light-emitting layers EML may be disposed in opening parts OH defined in a pixel defining layer PDL. For example, the light-emitting layers EML, which are separated by the pixel defining layer PDL and correspondingly provided to the light-emitting regions PXA-R, PXA-G and PXA-B, respectively, may emit light in a same wavelength range. In the display device DD-a according to an embodiment, the light-emitting layer EML may emit blue light. Although not shown in the drawings, in an embodiment, the light-emitting layer EML may be provided as a common layer for all light-emitting regions PXA-R, PXA-G, and PXA-B.

The light control layer CCL may be disposed on the display panel DP. The light control layer CCL may include a light converter. The light converter may be a quantum dot, a phosphor, or the like. The light converter may convert the wavelength of a provided light and may emit the resulting light. For example, the light control layer CCL may be a layer including a quantum dot or a layer including a phosphor.

The light control layer CCL may include light control parts CCP1, CCP2, and CCP3. The light control parts CCP1, CCP2, and CCP3 may be separated from each other.

Referring to FIG. 8, partition patterns BMP may be disposed between the separated light control parts CCP1, CCP2, and CCP3, but embodiments are not limited thereto. FIG. 8 illustrates that the partition patterns BMP do not overlap the light control parts CCP1, CCP2, and CCP3, but the edges of the light control parts CCP1, CCP2, and CCP3 may overlap at least a portion of the partition patterns BMP.

The light control layer CCL may include a first light control part CCP1 including a first quantum dot QD1 which converts first color light provided from the light-emitting element ED into second color light, a second light control part CCP2 including a second quantum dot QD2 which converts first color light into third color light, and a third light control part CCP3 transmitting the first color light.

In an embodiment, the first light control part CCP1 may provide red light which is the second color light, and the second light control part CCP2 may provide green light which is the third color light. The third light control part CCP3 may transmit blue light which is the first color light provided from the light-emitting element ED. For example, the first quantum dot QD1 may be a red quantum dot, and the second quantum dot QD2 may be a green quantum dot. The same description of quantum dots as provided above may be applied to the quantum dots QD1 and QD2.

The light control layer CCL may further include a scatterer SP. The first light control part CCP1 may include the first quantum dot QD1 and the scatterer SP, the second light control part CCP2 may include the second quantum dot QD2 and the scatterer SP, and the third light control part CCP3 may not include a quantum dot but may include the scatterer SP.

The scatterer SP may be an inorganic particle. For example, the scatterer SP may include at least one of TiO₂, ZnO, Al₂O₃, SiO₂, or hollow silica. The scatterer SP may include at least one of TiO₂, ZnO, Al₂O₃, SiO₂, or hollow silica, or the scatterer SP may be a mixture of two or more materials selected from TiO₂, ZnO, Al₂O₃, SiO₂, and hollow silica.

The first light control part CCP1, the second light control part CCP2, and the third light control part CCP3 may respectively include base resins BR1, BR2, and BR3 for dispersing the quantum dots QD1 and QD2 and the scatterer SP. In an embodiment, the first light control part CCP1 may include the first quantum dot QD1 and the scatterer SP dispersed in the first base resin BR1, the second light control part CCP2 may include the second quantum dot QD2 and the scatterer SP dispersed in the second base resin BR2, and the third light control part CCP3 may include the scatterer particle SP dispersed in the third base resin BR3. The base resins BR1, BR2, and BR3 may each be a medium in which the quantum dots QD1 and QD2 and the scatterer SP are dispersed, and may be composed of various resin compositions which may be generally referred to as a binder. For example, the base resins BR1, BR2, and BR3 may be acrylic resins, urethane-based resins, silicone-based resins, or epoxy-based resins, etc. The base resins BR1, BR2, and BR3 may be transparent resins. In an embodiment, the first base resin BR1, the second base resin BR2, and the third base resin BR3 may be the same as or different from each other.

The light control layer CCL may include a barrier layer BFL1. The barrier layer BFL1 may block the infiltration of moisture and/or oxygen (hereinafter, referred to as “moisture/oxygen”). The barrier layer BFL1 may be disposed on the light control parts CCP1, CCP2, and CCP3 to block the exposure of the light control parts CCP1, CCP2 and CCP3 to moisture/oxygen. A barrier layer BFL1 may cover the light control parts CCP1, CCP2, and CCP3. A barrier layer BFL2 may be provided between the light control parts CCP1, CCP2, and CCP3 and a color filter layer CFL.

The barrier layers BFL1 and BFL2 may include at least one inorganic layer. For example, the barrier layers BFL1 and BFL2 may be formed by including an inorganic material. For example, the barrier layers BFL1 and BFL2 may each independently include silicon nitride, aluminum nitride, zirconium nitride, titanium nitride, hafnium nitride, tantalum nitride, silicon oxide, aluminum oxide, titanium oxide, tin oxide, cerium oxide, silicon oxynitride, or a metal thin film with sufficient light transmittance, etc. The barrier layers BFL1 and BFL2 may further include an organic film. The barrier layers BFL1 and BFL2 may be formed of a single layer or of multiple layers.

In the display device DD-a according to an embodiment, the color filter layer CFL may be disposed on the light control layer CCL. In an embodiment, the color filter layer CFL may be disposed directly on the light control layer CCL. For example, the barrier layer BFL2 may be omitted.

The color filter layer CFL may include filters CF1, CF2, and CF3. The color filter layer CFL may include a first filter CF1 transmitting second color light, a second filter CF2 transmitting third color light, and a third filter CF3 transmitting first color light. For example, the first filter CF1 may be a red filter, the second filter CF2 may be a green filter, and the third filter CF3 may be a blue filter. Each of the filters CF1, CF2, and CF3 may include a polymer photosensitive resin and a pigment or dye. The first filter CF1 may include a red pigment or dye, the second filter CF2 may include a green pigment or dye, and the third filter CF3 may include a blue pigment or dye. However, embodiments are not limited thereto, and the third filter CF3 may not include a pigment or dye. The third filter CF3 may include a polymer photosensitive resin and may not include a pigment or dye. The third filter CF3 may be transparent. The third filter CF3 may be formed of a transparent photosensitive resin.

In an embodiment, the first filter CF1 and the second filter CF2 may each be a yellow filter. The first filter CF1 and the second filter CF2 may not be separated from each other and may be provided as a single filter. The first to third filters CF1, CF2, and CF3 may be respectively disposed corresponding to each of a red light-emitting region PXA-R, a green light-emitting region PXA-G, and a blue light-emitting region PXA-B.

Although not shown in the drawings, the color filter layer CFL may include a light blocking part (not shown). The color filter layer CFL may include a light blocking part (not shown) disposed to overlap the boundaries between the adjacent filters CF1, CF2, and CF3. The light blocking part (not shown) may be a black matrix. The light blocking part (not shown) may include an organic light blocking material or an inorganic light blocking material which includes a black pigment or a black dye. The light blocking part (not shown) may demarcate adjacent filters CF1, CF2, and CF3. In an embodiment, the light blocking part (not shown) may be formed of a blue filter.

A base substrate BL may be disposed on the color filter layer CFL. The base substrate BL may provide a base surface on which the color filter layer CFL and the light control layer CCL are disposed. The base substrate BL may be a glass substrate, a metal substrate, a plastic substrate, and the like. However, embodiments are not limited thereto, and the base substrate BL may include an inorganic layer, an organic layer, or a composite material layer. Although not shown in the drawings, in an embodiment, the base substrate BL may be omitted.

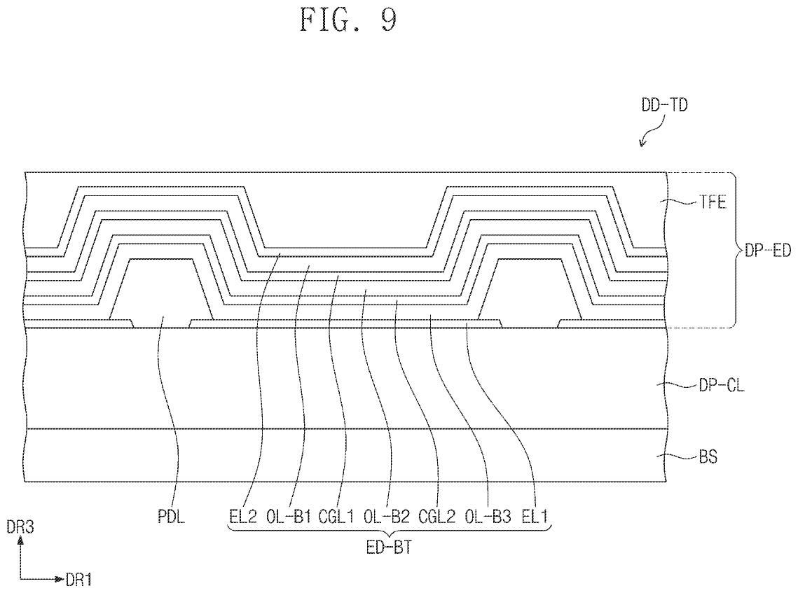

FIG. 9 is a schematic cross-sectional view illustrating a portion of the display device according to an embodiment. FIG. 9 illustrates a schematic cross-sectional view of a portion corresponding to the display panel DP in FIG. 8.

In a display device DD-TD according to an embodiment, a light-emitting element ED-BT may include light-emitting structures OL-B1, OL-B2, and OL-B3. The light-emitting element ED-BT may include first electrode EL1 and second electrode EL2 which face each other, and the light-emitting structures OL-B1, OL-B2, and OL-B3 stacked in a thickness direction and provided between the first electrode EL1 and the second electrode EL2. Each of the light-emitting structures OL-B1, OL-B2, and OL-B3 may include a light-emitting layer EML (FIG. 8), and a hole transport region HTR and an electron transport region ETR disposed with the light-emitting layer EML (FIG. 8) therebetween.

For example, the light-emitting element ED-BT included in the display device DD-TD according to an embodiment may be a light-emitting element having a tandem structure including multiple light-emitting layers.

In an embodiment illustrated in FIG. 9, light emitted from each of the light-emitting structures OL-B1, OL-B2, and OL-B3 may be all blue light. However, embodiments are not limited thereto, and the wavelength ranges of light emitted from the light-emitting structures OL-B1, OL-B2, and OL-B3 may be different from each other. For example, the light-emitting element ED-BT including the light-emitting structures OL-B1, OL-B2, and OL-B3 which emit light in different wavelength ranges may emit white light.

Charge generation layers CGL1 and CGL2 may be disposed between neighboring light-emitting structures OL-B1, OL-B2, and OL-B3. The charge generation layers CGL1 and CGL2 may each independently include a p-type charge generation layer and/or an n-type charge generation layer.

At least one of the light-emitting structures OL-B1, OL-B2, or OL-B3 included in the display device DD-TD according to an embodiment may include the above-described amine compound according to an embodiment.

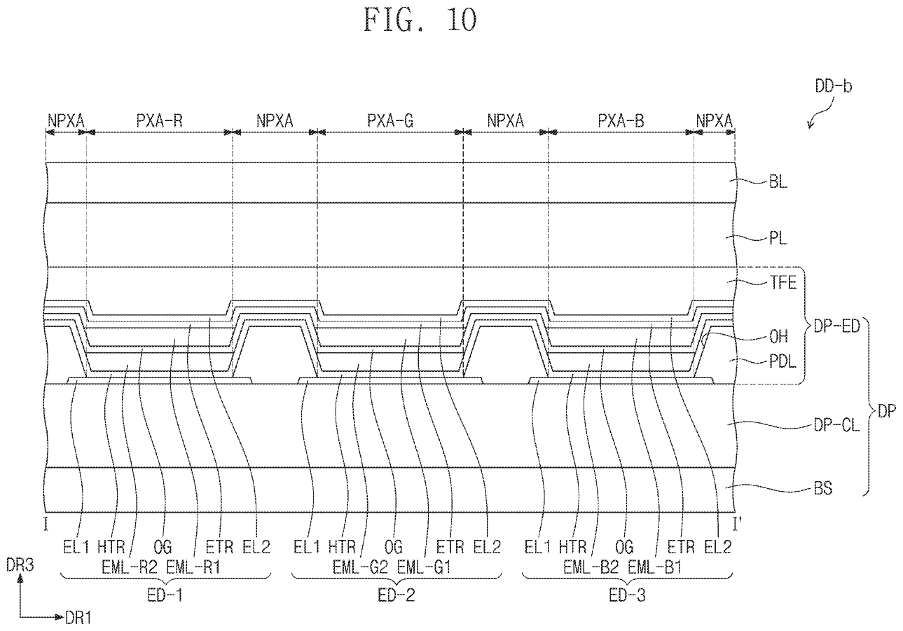

Referring to FIG. 10, a display device DD-b according to an embodiment may include light-emitting elements ED-1, ED-2, and ED-3 which each include two light-emitting layers that are stacked. The display device according to an embodiment illustrated in FIG. 10 may differ from the display device DD according to an embodiment illustrated in FIG. 2 at least in that the light-emitting elements ED-1, ED-2, and ED-3 in the display device in FIG. 10 each include two light-emitting layers which are stacked in a thickness direction. In each of the first to third light-emitting elements ED-1, ED-2, and ED-3, the two light-emitting layers may emit light of a same wavelength range.

The first light-emitting element ED-1 may include a first red light-emitting layer EML-R1 and a second red light-emitting layer EML-R2. The second light-emitting element ED-2 may include a first green light-emitting layer EML-G1 and a second green light-emitting layer EML-G2. The third light-emitting element ED-3 may include a first blue light-emitting layer EML-B1 and a second blue light-emitting layer EML-B2. A light-emitting auxiliary part OG may be disposed between the first red light-emitting layer EML-R1 and the second red light-emitting layer EML-R2, between the first green light-emitting layer EML-G1 and the second green light-emitting layer EML-G2, and between the first blue light-emitting layer EML-B1 and the second blue light-emitting layer EML-B2.

The light-emitting auxiliary part OG may be a single layer or multiple layers. The light-emitting auxiliary part OG may include a charge generation layer. For example, the light-emitting auxiliary part OG may include an electron transport region, a charge generation layer, and a hole transport region which are sequentially stacked. The light-emitting auxiliary part OG may be provided as a common layer for all of the first to third light-emitting elements ED-1, ED-2, and ED-3. However, embodiments are not limited thereto, and the light-emitting auxiliary part OG may be provided by being patterned in the openings OH defined in the pixel defining layer PDL.

The first red light-emitting layer EML-R1, the first green light-emitting layer EML-G1, and the first blue light-emitting layer EML-B1 may each be disposed between the electron transport region ETR and the light-emitting auxiliary part OG. The second red light-emitting layer EML-R2, the second green light-emitting layer EML-G2, and the second blue light-emitting layer EML-B2 may each be disposed between the light-emitting auxiliary part OG and the hole transport region HTR.

For example, the first light-emitting element ED-1 may include the first electrode EL1, the hole transport region HTR, the second red light-emitting layer EML-R2, the light-emitting auxiliary part OG, the first red light-emitting layer EML-R1, the electron transport region ETR, and the second electrode EL2, which are stacked in that order. The second light-emitting element ED-2 may include the first electrode EL1, the hole transport region HTR, the second green light-emitting layer EML-G2, the light-emitting auxiliary part OG, the first green light-emitting layer EML-G1, the electron transport region ETR, and the second electrode EL2, which are stacked in that order. The third light-emitting element ED-3 may include the first electrode EL1, the hole transport region HTR, the second blue light-emitting layer EML-B2, the light-emitting auxiliary part OG, the first blue light-emitting layer EML-B1, the electron transport region ETR, and the second electrode EL2, which are stacked in that order.

An optical auxiliary layer PL may be disposed on the display element layer DP-ED. The optical auxiliary layer PL may include a polarization layer. The optical auxiliary layer PL may be disposed on the display panel DP and may control light reflected at the display panel DP from an external light. Although not shown in the drawings, in an embodiment, the optical auxiliary layer PL may be omitted from the display device DD-b.

In contrast to FIGS. 9 and 10, FIG. 11 illustrates a display device DD-c that is different at least in that it includes four light-emitting structures OL-B1, OL-B2, OL-B3, and OL-C1. A light-emitting element ED-CT may include a first electrode EL1 and a second electrode EL2 which face each other, and the first to fourth light-emitting structures OL-B1, OL-B2, OL-B3, and OL-C1 which are stacked in a thickness direction and provided between the first electrode EL1 and the second electrode EL2. Charge generation layers CGL1, CGL2, and CGL3 may be disposed between the first to fourth light-emitting structures OL-B1, OL-B2, OL-B3, and OL-C1. Among the four light-emitting structures, the first to third light-emitting structures OL-B1, OL-B2, and OL-B3 may each emit blue light, and the fourth light-emitting structure OL-C1 may emit green light. However, embodiments are not limited thereto, and the first to fourth light-emitting structures OL-B1, OL-B2, OL-B3, and OL-C1 may emit light in wavelength ranges that are different from each other.

The charge generation layers CGL1, CGL2, and CGL3 which are disposed between the neighboring light-emitting structures OL-B1, OL-B2, OL-B3, and OL-C1 may each independently include a p-type charge generation layer and/or an n-type charge generation layer.

At least one of the light-emitting structures OL-B1, OL-B2, OL-B3, and OL-C1 included in the display device DD-c according to the embodiment may include the above-described amine compound according to an embodiment.

The light-emitting element ED according to an embodiment may include the above-described amine compound according to an embodiment in at least one functional layer disposed between the first electrode EL1 and the second electrode EL2, thereby exhibiting improved luminous efficiency and lifetime characteristics. The light-emitting element ED according to an embodiment may include the above-described amine compound according to an embodiment in at least one among the hole transport region HTR, the light-emitting layer EML, and the electron transport region ETR, or may include the described amine compound in the capping layer CPL.

For example, the amine compound according to an embodiment may be included in the hole transport region HTR of the light-emitting element ED according to an embodiment, and the light-emitting element according to an embodiment may exhibit excellent luminous efficiency and long lifetime characteristics.

The above-described amine compound according to an embodiment may exhibit improved lifetime characteristics by containing a dibenzoheterole moiety protected by a naphthyl group having excellent heat and charge resistance. In an embodiment, as the bonding position of the dibenzoheterole group and the naphthyl group is specified, the symmetry of the amine compound molecule is reduced, so that the crystallinity of the amine compound molecule is suppressed, and thus the hole transportability of the amine compound according to an embodiment may be improved. Accordingly, the efficiency of the light-emitting element including the amine compound according to an embodiment may be improved.

Hereinafter, an amine compound according to an embodiment and a light-emitting element according to an embodiment will be described in detail with reference to the Examples and the Comparative Examples. The Examples illustrated below are only provided as illustrations to assist in understanding the disclosure, and the scope thereof is not limited thereto.

### EXAMPLES

1. Synthesis of Amine Compound

A synthesis method for an amine compound according to embodiments will be explained by describing synthesis methods for Compounds 3, 5, 13, 19, 45, and 52. The synthesis method for an amine compound described below is only an example, and a synthesis method for a compound according to embodiments is not limited to the Examples.

(1) Synthesis of Compound 3

Aromatic Compound 3 according to Example may be synthesized, for example, by the process of Reaction Formulas below.

<Synthesis of Intermediate A>

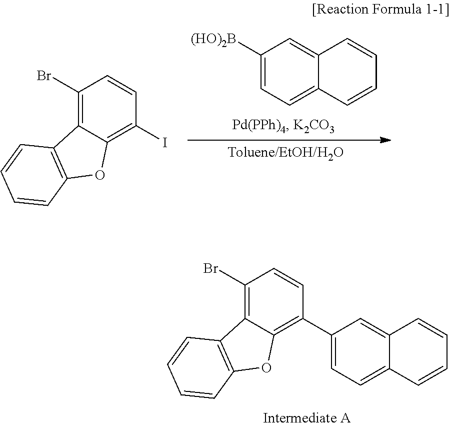 [SMILES: Brc1ccc(I)c2oc3ccccc3c12 ; OB(O)c1ccc2ccccc2c1 ; Brc1ccc(-c2ccc3ccccc3c2)c2oc3ccccc3c12]

1-bromo-4-iodobenzofuran (25 g, 67 mmol), 2-naphthalene boronic acid (312.6 g, 73.7 mmol), tetrakis(triphenylphosphine)palladium(0) (4.6 g, 4 mmol), and K₂CO₃ (28 g, 201 mmol) were added to a mixed solution of 268 mL of toluene, 134 mL of ethanol, and 67 mL of water, and after degassing, the resultant mixture was heated at 85° C. in an argon atmosphere for 2 hours. After cooling, the reaction solution was filtered through Florisil and concentrated, and the obtained residue was purified by column chromatography. After purification, Intermediate A (20 g, 53 mmol) was obtained.

<Synthesis of Intermediate B>

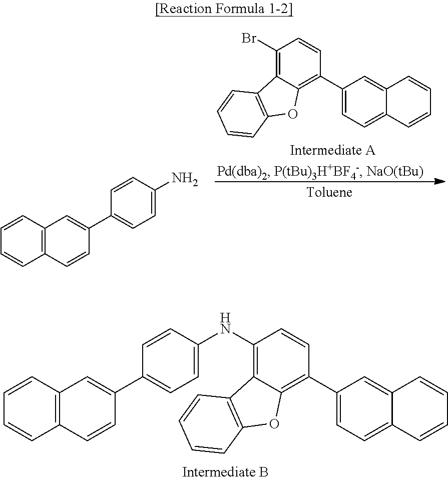 [SMILES: Brc1ccc(-c2ccc3ccccc3c2)c2oc3ccccc3c12 ; Nc1ccc(-c2ccc3ccccc3c2)cc1 ; c1ccc2cc(-c3ccc(Nc4ccc(-c5ccc6ccccc6c5)c5oc6ccccc6c45)cc3)ccc2c1]

After 4-(naphthalene-2-yl)aniline (8 g, 41 mmol), Intermediate A (16.7 g, 45 mmol), Pd(dba)₂ (1.17 g, 2.03 mmol), P(tBu)₃H⁺BF₄⁻ (2.36 g, 8.14 mmol), and NaO(tBu) (3.91 g, 40.68 mmol) were degassed, 407 mL of toluene was added dropwise to the reaction solution in an argon atmosphere and heated at 80° C. for 2 hours. After cooling, the reaction solution was filtered through Florisil and concentrated, and the obtained residue was purified by column chromatography to obtain Intermediate B (16.7 g, 33 mmol).

<Synthesis of Compound 3>

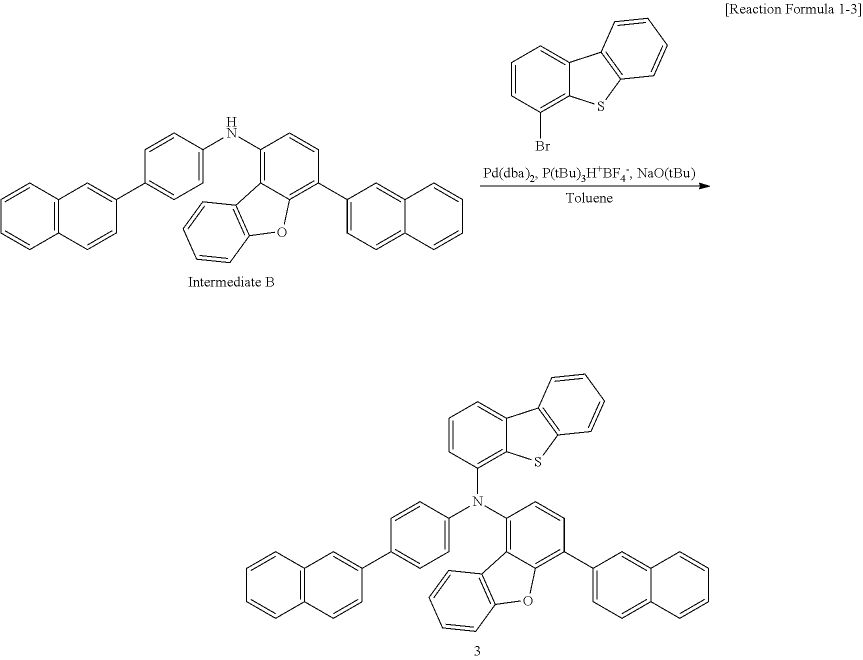 [SMILES: c1ccc2cc(-c3ccc(Nc4ccc(-c5ccc6ccccc6c5)c5oc6ccccc6c45)cc3)ccc2c1 ; Brc1cccc2c1sc1ccccc12 ; c1ccc2cc(-c3ccc(N(c4cccc5c4sc4ccccc45)c4ccc(-c5ccc6ccccc6c5)c5oc6ccccc6c45)cc3)ccc2c1]

After 4-bromodibenzothiophene (7.9 g, 30 mmol), Intermediate B (14 g, 27 mmol), Pd(dba)₂ (0.78 g, 1.36 mmol), P(tBu)₃H⁺BF₄⁻ (1.74 g, 6.0 mmol), and NaO(tBu) (5.22 g, 54.34 mmol) were degassed, 272 mL of toluene was added dropwise to the reaction solution in an argon atmosphere and heated at 80° C. for 3 hours. After cooling, the reaction solution was filtered through Florisil and concentrated, and the obtained residue was purified by column chromatography to obtain Compound 3 (13.8 g, 19.8 mmol).

The FAB-MS measurement showed a molecular ion peak (m/z=693.2), and it was thus confirmed that the obtained compound was Compound 3.

(2) Synthesis of Compound 5

Amine Compound 5 according to Example may be synthesized, for example, by the process of Reaction Formulas below.

<Synthesis of Intermediate C>

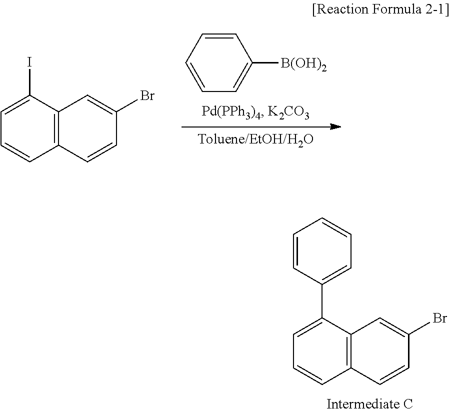 [SMILES: Brc1ccc2cccc(I)c2c1 ; OB(O)c1ccccc1 ; Brc1ccc2cccc(-c3ccccc3)c2c1]

2-bromo-8-iodonaphthalene (25 g, 75 mmol), phenyl boronic acid (10.1 g, 82.6 mmol), tetrakis(triphenylphosphine)palladium(0) (5.2 g, 4.5 mmol), and K₂CO₃ (31 g, 225 mmol) were added to a mixed solution of 300 mL of toluene, 150 mL of ethanol, and water, and after degassing, the resultant mixture was heated at 85° C. in an argon atmosphere for 2 hours. After cooling, the reaction solution was filtered through Florisil and concentrated, and the obtained residue was purified by column chromatography to obtain Intermediate C (17.4 g, 62 mmol).

<Synthesis of Intermediate D>

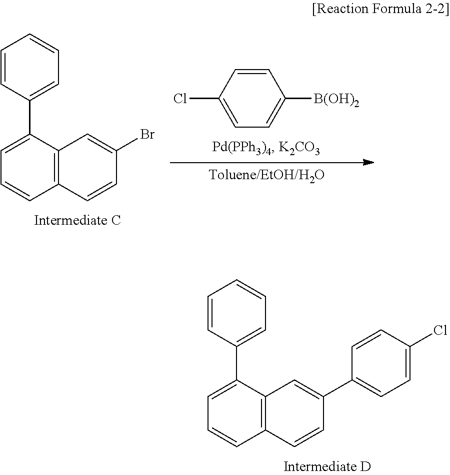 [SMILES: Brc1ccc2cccc(-c3ccccc3)c2c1 ; OB(O)c1ccc(Cl)cc1 ; Clc1ccc(-c2ccc3cccc(-c4ccccc4)c3c2)cc1]

Intermediate C (17 g, 60 mmol), 4-chlorophenyl boronic acid (10.3 g, 66 mmol), tetrakis(triphenylphosphine)palladium(0) (4.2 g, 3.6 mmol), and K₂CO₃ (24.9 g, 180 mmol) were added to a mixed solution of 240 mL of toluene, 120 mL of ethanol, and 60 mL of water, and after degassing, the resultant mixture was heated at 85° C. in an argon atmosphere for 2 hours. After cooling, the reaction solution was filtered through Florisil and concentrated, and the obtained residue was purified by column chromatography to obtain Intermediate D (14.2 g, 45 mmol).

<Synthesis of Intermediate E>

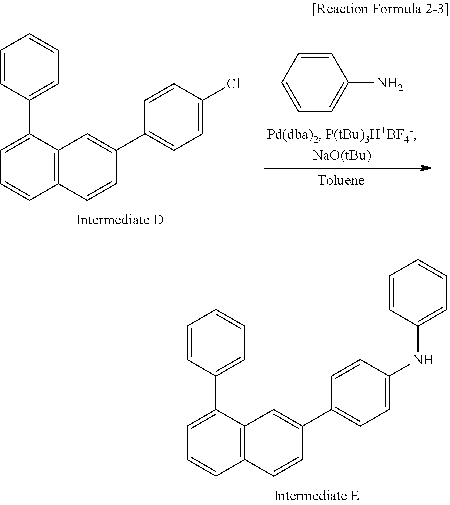 [SMILES: Clc1ccc(-c2ccc3cccc(-c4ccccc4)c3c2)cc1 ; Nc1ccccc1 ; c1ccc(Nc2ccc(-c3ccc4cccc(-c5ccccc5)c4c3)cc2)cc1]

After Intermediate D (10 g, 31.8 mmol), aniline (3.3 g, 34.9 mmol), Pd(dba)₂ (0.91 g, 1.59 mmol), P(tBu)₃H⁺BF₄⁻ (1.84 g, 6.35 mmol), and NaO(tBu) (3.05 g, 31.77 mmol) were degassed, 272 mL of toluene was added dropwise to the reaction solution in an argon atmosphere and heated at 80° C. for 3 hours. After cooling, the reaction solution was filtered through Florisil and concentrated, and the obtained residue was purified by column chromatography to obtain Intermediate E (8.37 g, 22.6 mmol).

<Synthesis of Compound 5>

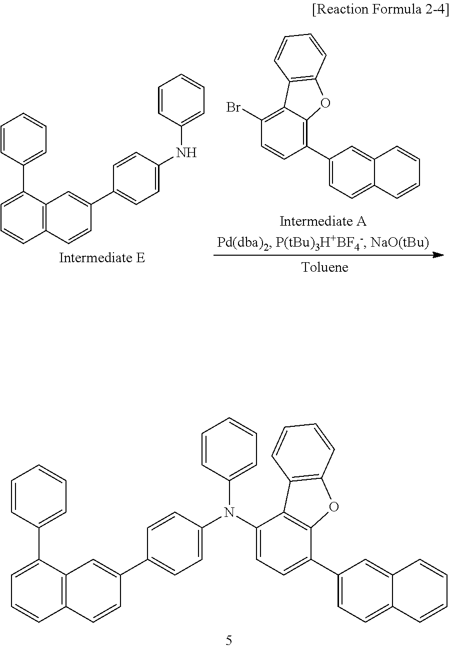

After Intermediate E (5 g, 13.5 mmol), Intermediate A (5.53 g, 14.8 mmol), Pd(dba)₂ (0.39 g, 0.67 mmol), P(tBu)₃H⁺BF₄⁻ (0.78 g, 2.69 mmol), and NaO(tBu) (1.29 g, 13.47 mmol) were degassed, 134 mL of toluene was added dropwise to the reaction solution in an argon atmosphere and heated at 80° C. for 3 hours. After cooling, the reaction solution was filtered through Florisil and concentrated, and the obtained residue was purified by column chromatography to obtain Compound 5 (5.01 g, 7.54 mmol, yield 56%). The FAB-MS measurement showed a molecular ion peak (m/z=663.3), and it was thus confirmed that the obtained compound was Compound 5.

(3) Synthesis of Compound 14

Compound 14 was synthesized in the same manner as in the synthesis method for Compound 1 except that 4-bromo-6-phenyl-dibenzothiophene was used instead of 4-bromodibenzothiophene in the synthesis example of Compound 1. Compound 14 was obtained at a yield of 32%. The FAB-MS measurement showed a molecular ion peak (m/z=769.2), and it was thus confirmed that the obtained compound was Compound 14.

(4) Synthesis of Compound 19

Compound 19 was synthesized in the same manner as in the synthesis method for Compound 1 except that 1-bromo-4-iodobenzothiophene instead of 1-bromo-4-iodobenzofuran and 4-bromodibenofuran instead of 4-bromodibenzothiophene were used in the synthesis example of Compound 1. Compound 19 was obtained at a yield of 48%. The FAB-MS measurement showed a molecular ion peak (m/z=693.3), and it was thus confirmed that the obtained compound was Compound 19.

(5) Synthesis of Compound 45

Compound 45 was synthesized in the same manner as in the synthesis method for Compound 2 except that 1-bromo-8-iodonaphthalene instead of 2-bromo-8-iodonaphthalene and 1-naphthyl amine instead of aniline were used in the synthesis example of Compound 2. Compound 45 was obtained at a yield of 51%. The FAB-MS measurement showed a molecular ion peak (m/z=713.3), and it was thus confirmed that the obtained compound was Compound 45.

(6) Synthesis of Compound 52

Compound 52 was synthesized in the same manner as in the synthesis method for Compound 1 except that 4-bromo-1-iodobenzothiophene instead of 1-bromo-4-iodobenzofuran and 3-bromodibenofuran instead of 4-bromodibenzothiophene were used in the synthesis example of Compound 1. Compound 52 was obtained at a yield of 41%. The FAB-MS measurement showed a molecular ion peak (m/z=693.2), and it was thus confirmed that the obtained compound was Compound 52.

(7) Synthesis of Compound 67

Compound 67 was synthesized in the same manner as in the synthesis method for Compound 1 except that 1-bromo-4-iodo-9-phenyl-carbazole was used instead of 1-bromo iodobenzofuran in the synthesis example of Compound 1. Compound 67 was obtained at a yield of 18%. The FAB-MS measurement showed a molecular ion peak (m/z=768.3), and it was thus confirmed that the obtained compound was Compound 67.

(8) Synthesis of Compound 81

Amine Compound 81 according to Example may be synthesized, for example, by the process of Reaction Formulas below.

<Synthesis of Intermediate F>

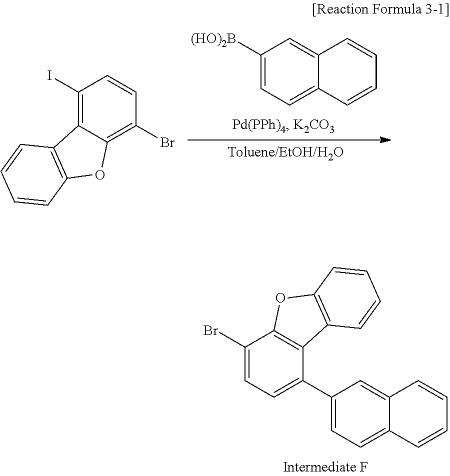 [SMILES: Brc1ccc(I)c2c1oc1ccccc12 ; OB(O)c1ccc2ccccc2c1 ; Brc1ccc(-c2ccc3ccccc3c2)c2c1oc1ccccc12]

4-bromo-1-iodobenzofuran (25 g, 67 mmol), 2-naphthalene boronic acid (312.6 g, 73.7 mmol), tetrakis(triphenylphosphine)palladium(0) (4.6 g, 4 mmol), and K₂CO₃ (28 g, 201 mmol) were added to a mixed solution of 268 mL of toluene, 134 mL of ethanol, and 67 mL of water, and after degassing, the resultant mixture was heated at 85° C. in an argon atmosphere for 2 hours. After cooling, the reaction solution was filtered through Florisil and concentrated, and the obtained residue was purified by column chromatography. After purification, Intermediate F (17.5 g, 47 mmol) was obtained.

<Synthesis of Compound 81>

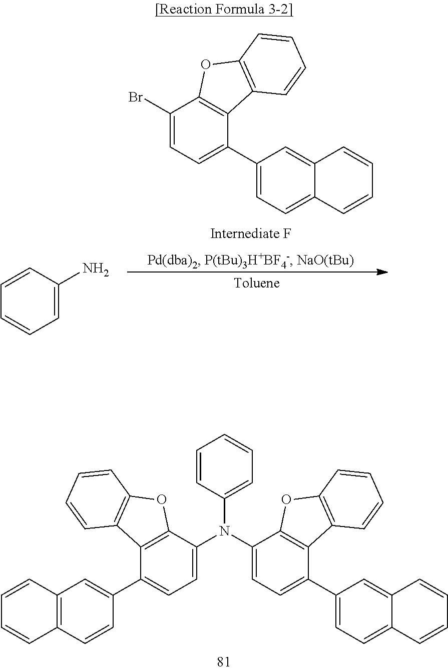 [SMILES: Brc1ccc(-c2ccc3ccccc3c2)c2c1oc1ccccc12 ; Nc1ccccc1 ; c1ccc(N(c2ccc(-c3ccc4ccccc4c3)c3c2oc2ccccc23)c2ccc(-c3ccc4ccccc4c3)c3c2oc2ccccc23)cc1]

After aniline(4 g, 43 mmol), Intermediate F(35.3 g, 94.6 mmol), Pd(dba)₂ (1.24 g, 2.15 mmol), P(tBu)₃H⁺BF₄⁻ (2.5 g, 8.6 mmol), and NaO(tBu) (4.13 g, 43.0 mmol) were degassed, 430 mL of toluene was added dropwise to the reaction solution in an argon atmosphere and heated at 80° C. for 2 hours. After cooling, the reaction solution was filtered through Florisil and concentrated, and the obtained residue was purified by column chromatography to obtain Compound 81 (14.5 g, 35.2 mmol). The FAB-MS measurement showed a molecular ion peak (m/z=677.2), and it was thus confirmed that the obtained compound was Compound 81.

2. Manufacture and Evaluation of Light-Emitting Element

Evaluation of light-emitting elements including compounds of Examples and Comparative Examples in a hole transport layer was conducted as described below. A method of manufacturing a light-emitting element for element evaluation is described below.

(1) Manufacture of Light-Emitting Element

An ITO-patterned glass substrate having a thickness of 1,500 Å was subjected to ultrasonic cleaning by using isopropyl alcohol for about five minutes and using pure water for about five minutes. After ultrasonic cleaning, the glass substrate was irradiated with UV rays for 30 minutes and subjected to ozone treatment. Thereafter, Compound HT1 and the HIL compound were deposited to a thickness of 100 Å at a weight ratio of 98:2 to form a hole injection layer.

Compound HT1 was deposited to a thickness of 1,200 Å to form a first hole transport layer. In Examples 1 to 8 and Comparative Examples 1 to 5, an Example Compound or a Comparative Example Compound was deposited to a thickness of 100 Å to form a second hole transport layer.

Thereafter, the host (E2) and the dopant (BD) were co-deposited at a weight ratio of 98:2 to form a light-emitting layer having a thickness of 300 Å. ET1 at a thickness of 100 Å and ET2 at a thickness of 200 Å were sequentially deposited to form an electron transport layer, and LiF was deposited to a thickness of 10 Å to form an electron injection layer.

A second electrode was formed to a thickness of 1,200 Å by using Ag and Mg at a weight ratio of 10:90.

In Examples, the hole injection layer, the hole transport layer, the light-emitting layer, the electron transport layer, the electron injection layer, and the second electrode were formed using a vacuum deposition device.

Example Compounds and Comparative Example Compounds used for manufacturing the light-emitting element are as follows.

### Example Compound

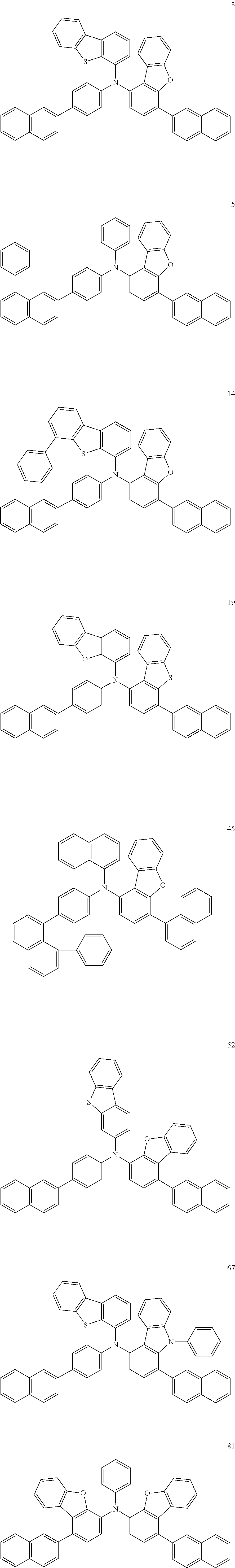

### Comparative Example Compound

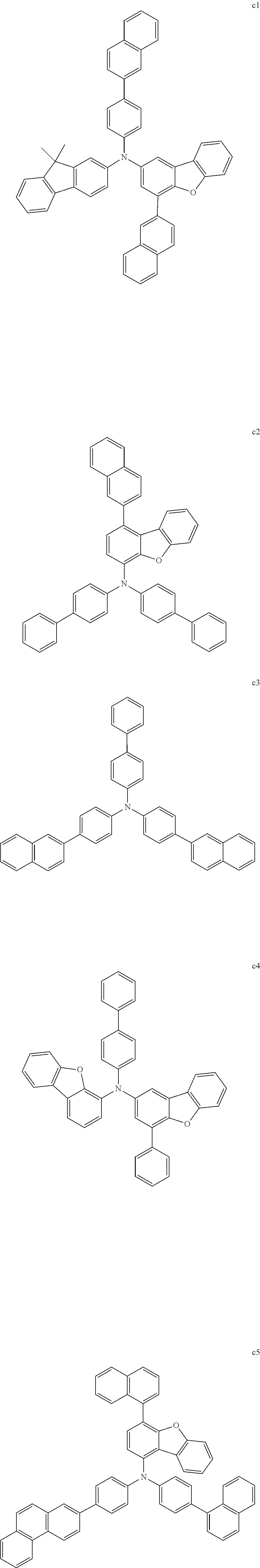 [SMILES: CC1(C)c2ccccc2-c2ccc(N(c3ccc(-c4ccc5ccccc5c4)cc3)c3cc(-c4ccc5ccccc5c4)c4oc5ccccc5c4c3)cc21 ; c1ccc(-c2ccc(N(c3ccc(-c4ccccc4)cc3)c3ccc(-c4ccc5ccccc5c4)c4c3oc3ccccc34)cc2)cc1 ; c1ccc(-c2ccc(N(c3ccc(-c4ccc5ccccc5c4)cc3)c3ccc(-c4ccc5ccccc5c4)cc3)cc2)cc1 ; c1ccc(-c2ccc(N(c3cc(-c4ccccc4)c4oc5ccccc5c4c3)c3cccc4c3oc3ccccc34)cc2)cc1 ; c1ccc2c(-c3ccc(N(c4ccc(-c5ccc6c(ccc7ccccc76)c5)cc4)c4ccc(-c5cccc6ccccc56)c5oc6ccccc6c45)cc3)cccc2c1]

Compounds of each functional layer are as follows:

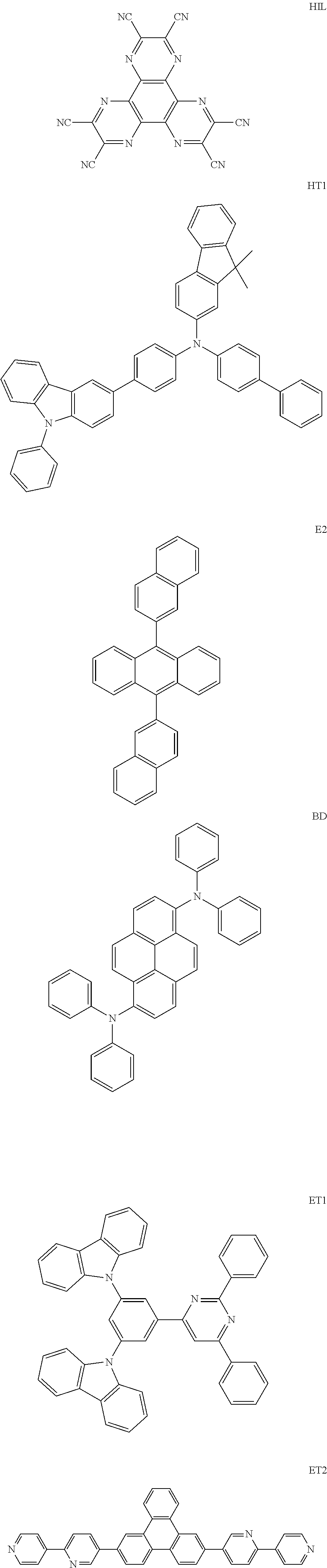

(2) Evaluation of Light-Emitting Element

Table 1 shows the evaluation results of the light-emitting elements according to Examples 1 to 8, and Comparative Examples 1 to 5. Table 1 show the luminous efficiency and half-life of the manufactured light-emitting elements through comparison between Examples and Comparative Examples. In the characteristic evaluation results for Examples and Comparative Examples shown in Table 1, luminous efficiency represents an efficiency value at a current density of 25 mA/cm², and lifetime represents a luminance half-life at 10 mA/cm².

In Table 1, the luminous efficiency and half-life characteristics are shown as relative values obtained when the luminous efficiency and lifetime of Comparative Example 1 are each 100%.

TABLE 1

| Manufacture | | | |
|---|---|---|---|
| Example of | Material for Second | Luminous | |
| Element | Hole Transport Layer | Efficiency | Lifetime |
| Example 1 | Compound 3 | 108% | 115% |
| Example 2 | Compound 5 | 113% | 121% |
| Example 3 | Compound 14 | 110% | 132% |
| Example 4 | Compound 19 | 107% | 120% |
| Example 5 | Compound 45 | 111% | 113% |
| Example 6 | Compound 52 | 115% | 110% |
| Example 7 | Compound 67 | 112% | 124% |
| Example 8 | Compound 81 | 109% | 116% |
| Comparative | Comparative Example | 100% | 100% |
| Example 1 | Compound c1 | | |
| Comparative | Comparative Example | 94% | 90% |
| Example 2 | Compound c2 | | |
| Comparative | Comparative Example | 91% | 97% |
| Example 3 | Compound c3 | | |
| Comparative | Comparative Example | 92% | 64% |
| Example 4 | Compound c4 | | |
| Comparative | Comparative Example | 92% | 96% |
| Example 5 | Compound c5 | | |

Referring to the results of Table 1, it may be confirmed that Examples in which the light-emitting element uses the amine compound according to an embodiment as a material for the hole transport layer exhibits excellent luminous efficiency and improved element lifetime characteristics. Referring to Table 1, it may be seen that the light-emitting elements of Examples 1 to 8 exhibit long lifetime and high efficiency characteristics as compared to the light-emitting elements of Comparative Examples 1 to 5. Since the amine compound according to Examples includes a dibenzoheterole moiety in which a naphthyl group is substituted for, it may be confirmed that high efficiency and long lifetime characteristics are exhibited as compared to the amine compound of Comparative Example. In amine compounds used in Examples 1 to 8, since a naphthyl group is substituted at the 4th position of dibenzoheterole, the hetero atom is protected by the naphthyl group and is less affected by electrons. Therefore, it is considered that the lifetime of the light-emitting element using the amine compounds of Examples is improved. In the amine compound used in Examples, the substitution position of the naphthyl group substituted at dibenzoheterole is the para position with respect to the nitrogen atom of the amine, so that the symmetry of the molecule of the amine compound is lowered and thus the crystallinity is suppressed to improve hole transportability. Accordingly, the light-emitting elements according to an embodiment are considered to exhibit high luminous efficiency characteristics.

Compared with Examples, Comparative Example Compound c1 used in Comparative Example 1 has, similarly to the amine compound of Examples, a dibenzoheterole moiety in which a naphthyl group is substituted for. However, Comparative Example Compound c1 is different from the compounds of Examples in terms of the substitution position of the naphthyl group because the naphthyl group is substituted for the nitrogen atom of the amine compound at the meta position of the dibenzoheterole moiety. Due to the difference in the substitution position of the naphthyl group, in Comparative Example Compound c1, a highest occupied molecular orbital (HOMO) is not sufficiently wide, and the electron density on the amine side is relatively increased. Accordingly, the deterioration of the compound is accelerated, so it is considered that Comparative Example Compound c1 exhibits lower lifetime characteristics than Example Compounds.

Comparative Example Compound c2 used in Comparative Example 2 is an amine compound having, similarly to Example Compounds, a dibenzoheterole moiety in which a naphthyl group is substituted for, but is different from Example Compounds in that a naphthyl group is not included in other substituents of the amine. Accordingly, it is considered that the hetero atom of the dibenzoheterole moiety in Comparative Example Compound c2 is not sufficiently protected by the naphthyl group, and thus is easily affected by electrons, resulting in a decrease in luminous efficiency and element lifetime.

Comparative Example Compound c3 used in Comparative Example 3 does not include a dibenzoheterole moiety and thus has a high molecular symmetry, and thus the crystallinity is increased. Thus, it is considered that the luminous efficiency and lifetime of Comparative Example 3 are reduced. Comparative Example 4 uses Comparative Example Compound c4 that does not include a naphthyl group, and has insufficient hole transport properties, thereby exhibiting significantly reduced lifetime characteristics.

Comparative Example Compound c5 used in Comparative Example 5 includes a phenanthryl group as one of the substituents of the amine compound, and the crystallinity is increased due to the high planarity of the phenanthryl group. Thus, it is considered that the luminous efficiency and lifetime of Comparative Example Compound 5 are reduced.

The amine compound according to an embodiment includes at least one dibenzoheterole moiety in which a naphthyl group is substituted for, and has a structure in which a naphthyl group is substituted at a position capable of protecting the dibenzoheterole moiety. Therefore, when the amine compound is used as a material for a light-emitting element, the luminous efficiency and element lifetime of the light-emitting element may be improved.

A light-emitting element according to an embodiment may include an amine compound according to an embodiment and thus exhibit high efficiency and long lifetime characteristics.

An amine compound according to an embodiment may be used as a material for achieving improved characteristics of a light-emitting element having high efficiency and long lifetime.

Embodiments have been disclosed herein, and although terms are employed, they are used and are to be interpreted in a generic and descriptive sense only and not for purpose of limitation. In some instances, as would be apparent by one of ordinary skill in the art, features, characteristics, and/or elements described in connection with an embodiment may be used singly or in combination with features, characteristics, and/or elements described in connection with other embodiments unless otherwise specifically indicated. Accordingly, it will be understood by those of ordinary skill in the art that various changes in form and details may be made without departing from the spirit and scope of the disclosure as set forth in the following claims.

What is claimed is:

1. A light-emitting element comprising:
a first electrode;
a second electrode disposed on the first electrode; and
at least one functional layer disposed between the first electrode and the second electrode and including an amine compound represented by Formula 1: 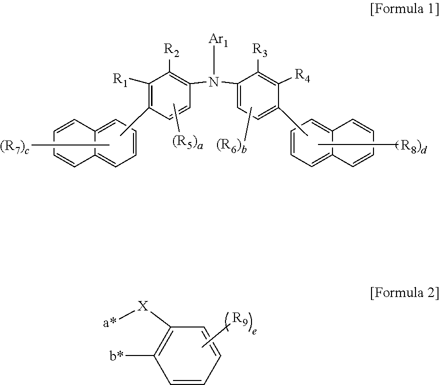
wherein in Formula 1,
Ar₁ is a substituted or unsubstituted aryl group having 6 to 40 ring-forming carbon atoms other than a phenanthryl group, or a substituted or unsubstituted heteroaryl group having 5 to 40 ring-forming carbon atoms,
R₁ to R₄ are each independently a hydrogen atom, a deuterium atom, a substituted or unsubstituted alkyl group having 1 to 20 carbon atoms, a substituted or unsubstituted aryl group having 6 to 40 ring-forming carbon atoms, or a single bond forming a ring by bonding with a group represented by Formula 2, and
a pair of R₁ and R₂ and/or a pair of R₃ and R₄ are bonded with a group represented by Formula 2 to form a ring,
wherein in Formula 2,
X is O, S, N(R₁₀), or C(R₁₁)(R₁₂), and
a* and b* each indicate a bond to one of R₁ to R₄ in Formula 1, and
wherein in Formula 1 and Formula 2,
R₅ to R₁₂ are each independently a hydrogen atom, a deuterium atom, a substituted or unsubstituted alkyl group having 1 to 20 carbon atoms, or a substituted or unsubstituted aryl group having 6 to 40 ring-forming carbon atoms,
a and b are each independently an integer from 0 to 2,
c and d are each independently an integer from 0 to 7, and
e is an integer from 0 to 4.

2. The light-emitting element of claim 1, wherein
the at least one functional layer comprises:
a light-emitting layer;
a hole transport region disposed between the first electrode and the light-emitting layer; and
an electron transport region disposed between the light-emitting layer and the second electrode, and
the hole transport region includes the amine compound.

3. The light-emitting element of claim 2, wherein
the hole transport region comprises at least one of a hole injection layer, a hole transport layer, or an electron blocking layer, and
the at least one of the hole injection layer, the hole transport layer, or the electron blocking layer includes the amine compound.

4. The light-emitting element of claim 2, wherein
the hole transport region comprises a first hole transport layer and a second hole transport layer which are sequentially stacked between the first electrode and the light-emitting layer,
the first hole transport layer and the second hole transport layer include different hole transport materials, and
the second hole transport layer includes the amine compound.

5. The light-emitting element of claim 1, wherein the amine compound represented by Formula 1 is represented by one of Formulas 1-1 to 1-3: 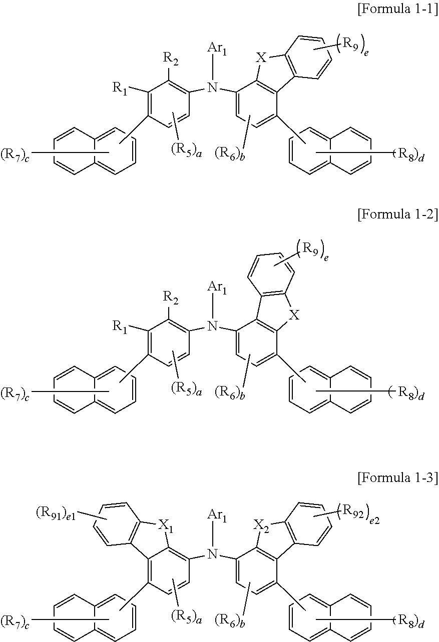
wherein in Formula 1-3,
X₁ and X₂ are each independently O, S, N(R₁₀), or C(R₁₁)(R₁₂),
e1 and e2 are each independently an integer from 0 to 4, and
R₉₁ and R₉₂ are each independently a hydrogen atom, a deuterium atom, a substituted or unsubstituted alkyl group having 1 to 20 carbon atoms, or a substituted or unsubstituted aryl group having 6 to 40 ring-forming carbon atoms, and
wherein in Formulas 1-1 to 1-3,
Ar₁, X, R₁ to R₁₂, and a to e are the same as defined in Formulas 1 and 2.

6. The light-emitting element of claim 5, wherein in Formula 1-3,
X₁ and X₂ are the same.

7. The light-emitting element of claim 1, wherein the group represented by Formula 2 is a group represented by one of Formulas 2-1 to 2-5: 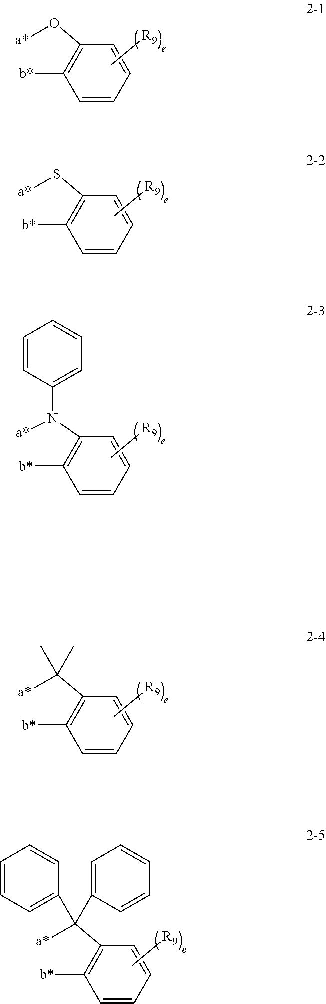
wherein in Formulas 2-1 to 2-5,
R₉, e, a*, and b* are the same as defined in Formula 2.

8. The light-emitting element of claim 1, wherein in Formula 1,
Ar₁ is a substituted or unsubstituted phenyl group, a substituted or unsubstituted naphthyl group, a substituted or unsubstituted dibenzothiophene group, or a substituted or unsubstituted dibenzofuran group.

9. The light-emitting element of claim 1, wherein in Formula 1,
at least one of R₁ to R₉ and Ar₁ is a deuterium atom or a substituent including a deuterium atom.

10. The light-emitting element of claim 2, wherein the light-emitting layer comprises a compound represented by Formula E-1: 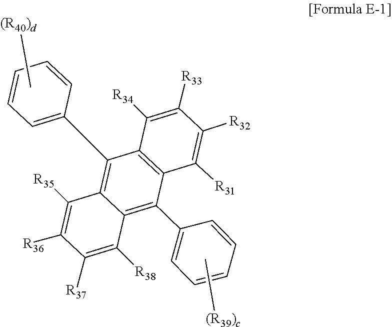
wherein in Formula E-1,
c and d are each independently an integer from 0 to 5,
R₃₁ to R₄₀ are each independently a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, or are bonded to an adjacent group to form a ring.

11. The light-emitting element of claim 1, further comprising:
a capping layer disposed on the second electrode, wherein
the capping layer has a refractive index equal to or greater than about 1.6.

12. The light-emitting element of claim 1, wherein the amine compound is selected from Compound Group 1: 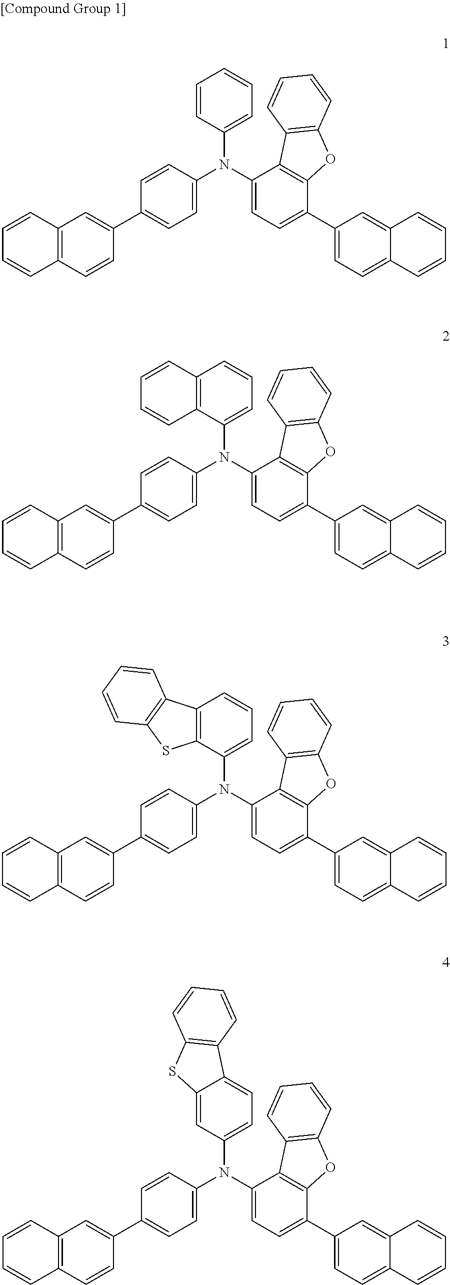 [SMILES: c1ccc(N(c2ccc(-c3ccc4ccccc4c3)cc2)c2ccc(-c3ccc4ccccc4c3)c3oc4ccccc4c23)cc1 ; c1ccc2cc(-c3ccc(N(c4cccc5ccccc45)c4ccc(-c5ccc6ccccc6c5)c5oc6ccccc6c45)cc3)ccc2c1 ; c1ccc2cc(-c3ccc(N(c4cccc5c4sc4ccccc45)c4ccc(-c5ccc6ccccc6c5)c5oc6ccccc6c45)cc3)ccc2c1 ; c1ccc2cc(-c3ccc(N(c4ccc5c(c4)sc4ccccc45)c4ccc(-c5ccc6ccccc6c5)c5oc6ccccc6c45)cc3)ccc2c1] 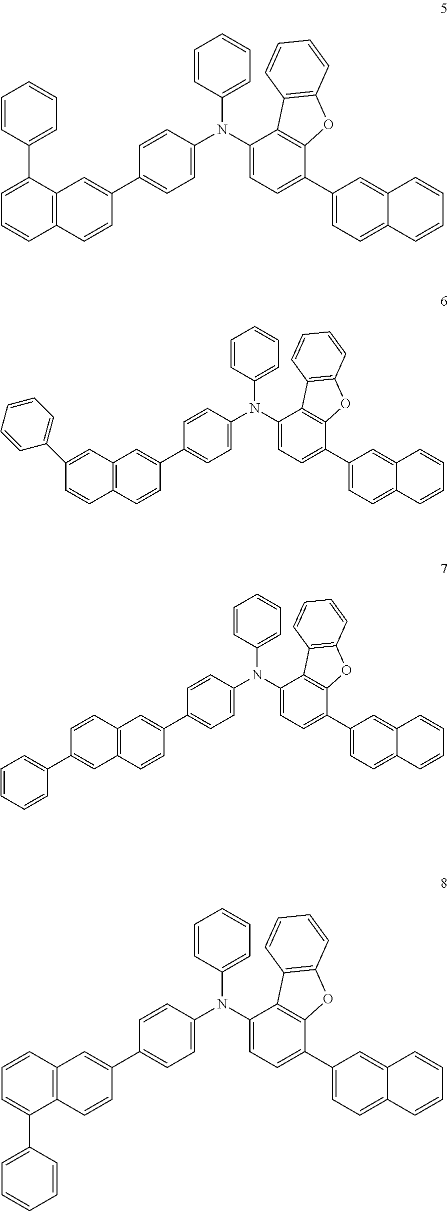 [SMILES: c1ccc(-c2cccc3ccc(-c4ccc(N(c5ccccc5)c5ccc(-c6ccc7ccccc7c6)c6oc7ccccc7c56)cc4)cc23)cc1 ; c1ccc(-c2ccc3ccc(-c4ccc(N(c5ccccc5)c5ccc(-c6ccc7ccccc7c6)c6oc7ccccc7c56)cc4)cc3c2)cc1 ; c1ccc(-c2ccc3cc(-c4ccc(N(c5ccccc5)c5ccc(-c6ccc7ccccc7c6)c6oc7ccccc7c56)cc4)ccc3c2)cc1 ; c1ccc(-c2cccc3cc(-c4ccc(N(c5ccccc5)c5ccc(-c6ccc7ccccc7c6)c6oc7ccccc7c56)cc4)ccc23)cc1] 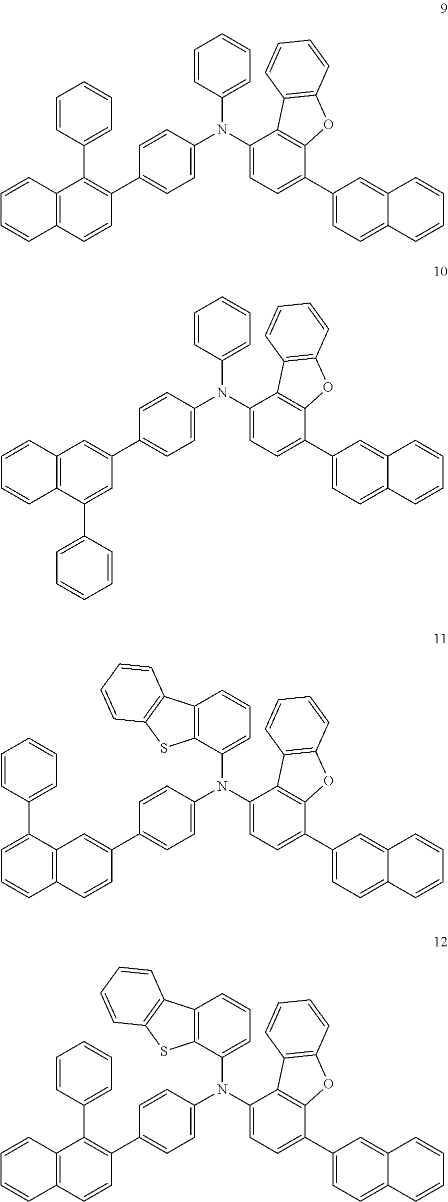 [SMILES: c1ccc(-c2c(-c3ccc(N(c4ccccc4)c4ccc(-c5ccc6ccccc6c5)c5oc6ccccc6c45)cc3)ccc3ccccc23)cc1 ; c1ccc(-c2cc(-c3ccc(N(c4ccccc4)c4ccc(-c5ccc6ccccc6c5)c5oc6ccccc6c45)cc3)cc3ccccc23)cc1 ; c1ccc(-c2cccc3ccc(-c4ccc(N(c5cccc6c5sc5ccccc56)c5ccc(-c6ccc7ccccc7c6)c6oc7ccccc7c56)cc4)cc23)cc1 ; c1ccc(-c2c(-c3ccc(N(c4cccc5c4sc4ccccc45)c4ccc(-c5ccc6ccccc6c5)c5oc6ccccc6c45)cc3)ccc3ccccc23)cc1] 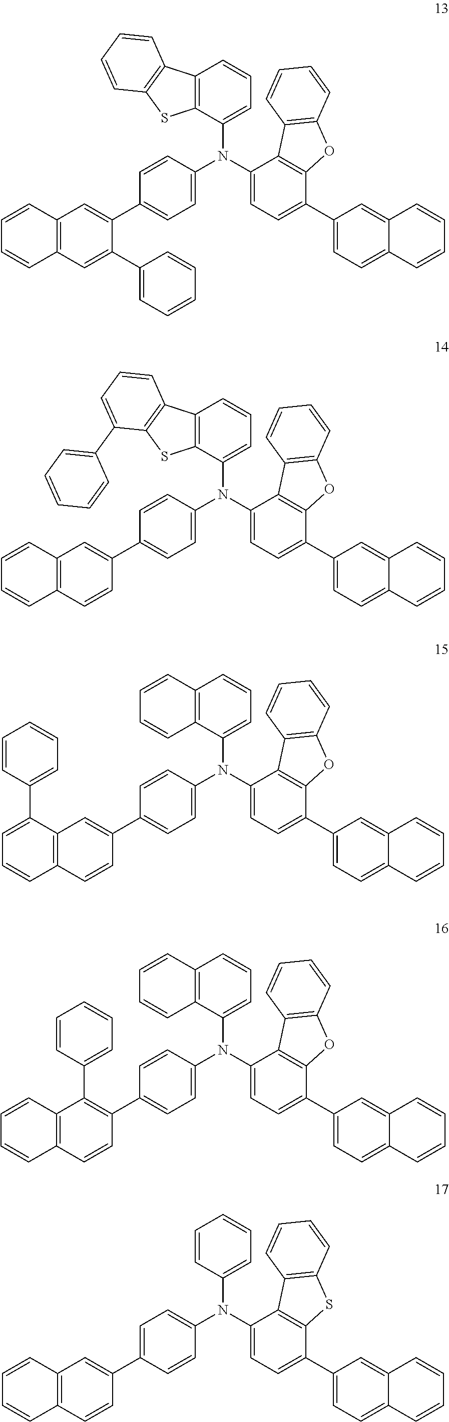 [SMILES: c1ccc(-c2cc3ccccc3cc2-c2ccc(N(c3cccc4c3sc3ccccc34)c3ccc(-c4ccc5ccccc5c4)c4oc5ccccc5c34)cc2)cc1 ; c1ccc(-c2cccc3c2sc2c(N(c4ccc(-c5ccc6ccccc6c5)cc4)c4ccc(-c5ccc6ccccc6c5)c5oc6ccccc6c45)cccc23)cc1 ; c1ccc(-c2cccc3ccc(-c4ccc(N(c5cccc6ccccc56)c5ccc(-c6ccc7ccccc7c6)c6oc7ccccc7c56)cc4)cc23)cc1 ; c1ccc(-c2c(-c3ccc(N(c4cccc5ccccc45)c4ccc(-c5ccc6ccccc6c5)c5oc6ccccc6c45)cc3)ccc3ccccc23)cc1 ; c1ccc(N(c2ccc(-c3ccc4ccccc4c3)cc2)c2ccc(-c3ccc4ccccc4c3)c3sc4ccccc4c23)cc1] 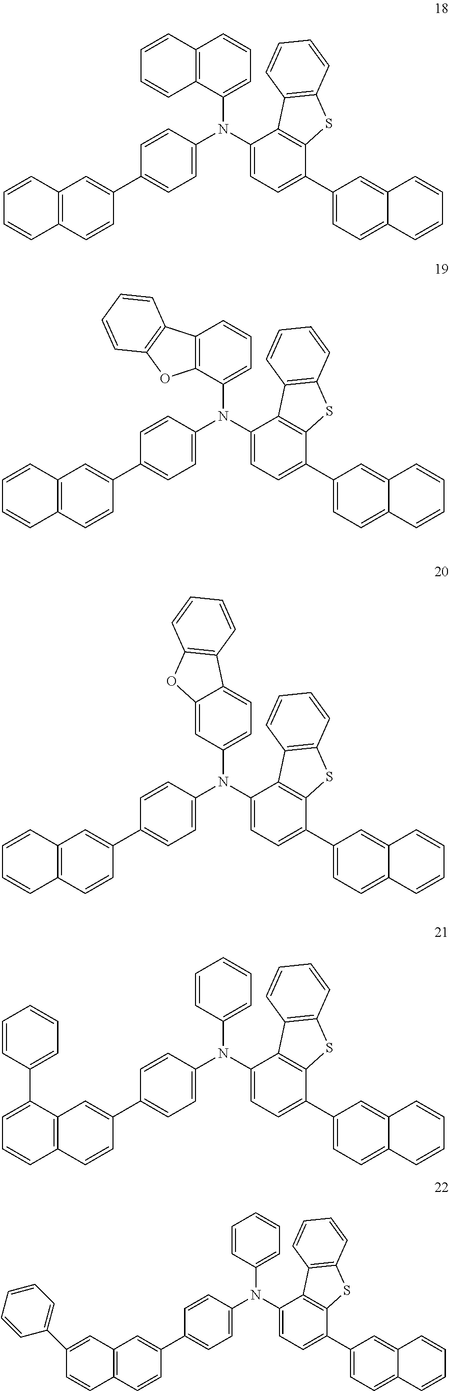 [SMILES: c1ccc2cc(-c3ccc(N(c4cccc5ccccc45)c4ccc(-c5ccc6ccccc6c5)c5sc6ccccc6c45)cc3)ccc2c1 ; c1ccc2cc(-c3ccc(N(c4cccc5c4oc4ccccc45)c4ccc(-c5ccc6ccccc6c5)c5sc6ccccc6c45)cc3)ccc2c1 ; c1ccc2cc(-c3ccc(N(c4ccc5c(c4)oc4ccccc45)c4ccc(-c5ccc6ccccc6c5)c5sc6ccccc6c45)cc3)ccc2c1 ; c1ccc(-c2cccc3ccc(-c4ccc(N(c5ccccc5)c5ccc(-c6ccc7ccccc7c6)c6sc7ccccc7c56)cc4)cc23)cc1 ; c1ccc(-c2ccc3ccc(-c4ccc(N(c5ccccc5)c5ccc(-c6ccc7ccccc7c6)c6sc7ccccc7c56)cc4)cc3c2)cc1] 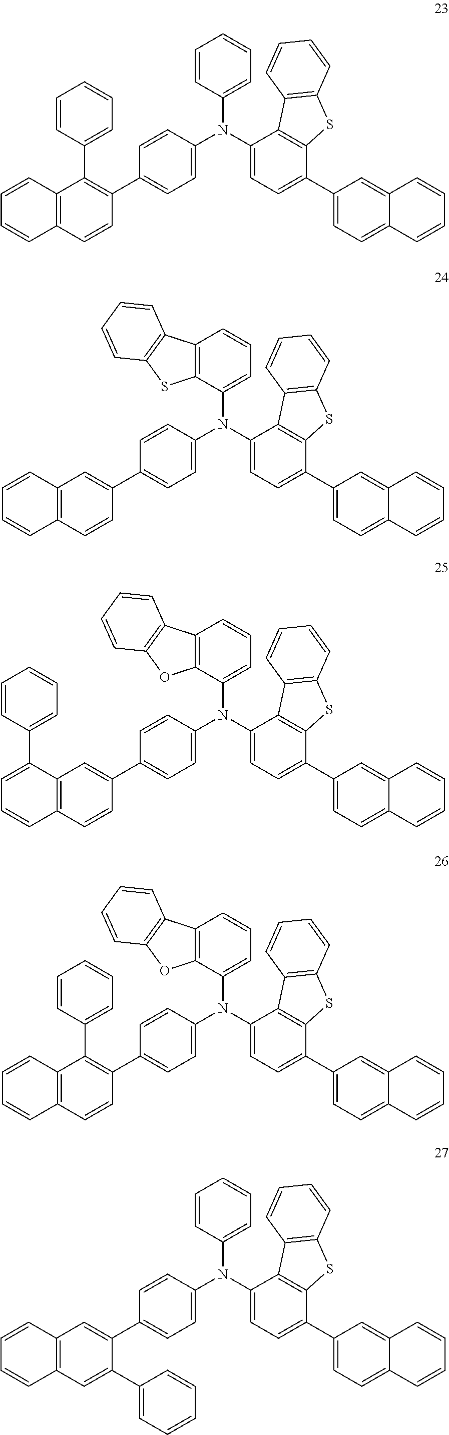 [SMILES: c1ccc(-c2c(-c3ccc(N(c4ccccc4)c4ccc(-c5ccc6ccccc6c5)c5sc6ccccc6c45)cc3)ccc3ccccc23)cc1 ; c1ccc2cc(-c3ccc(N(c4cccc5c4sc4ccccc45)c4ccc(-c5ccc6ccccc6c5)c5sc6ccccc6c45)cc3)ccc2c1 ; c1ccc(-c2cccc3ccc(-c4ccc(N(c5cccc6c5oc5ccccc56)c5ccc(-c6ccc7ccccc7c6)c6sc7ccccc7c56)cc4)cc23)cc1 ; c1ccc(-c2c(-c3ccc(N(c4cccc5c4oc4ccccc45)c4ccc(-c5ccc6ccccc6c5)c5sc6ccccc6c45)cc3)ccc3ccccc23)cc1 ; c1ccc(-c2cc3ccccc3cc2-c2ccc(N(c3ccccc3)c3ccc(-c4ccc5ccccc5c4)c4sc5ccccc5c34)cc2)cc1] 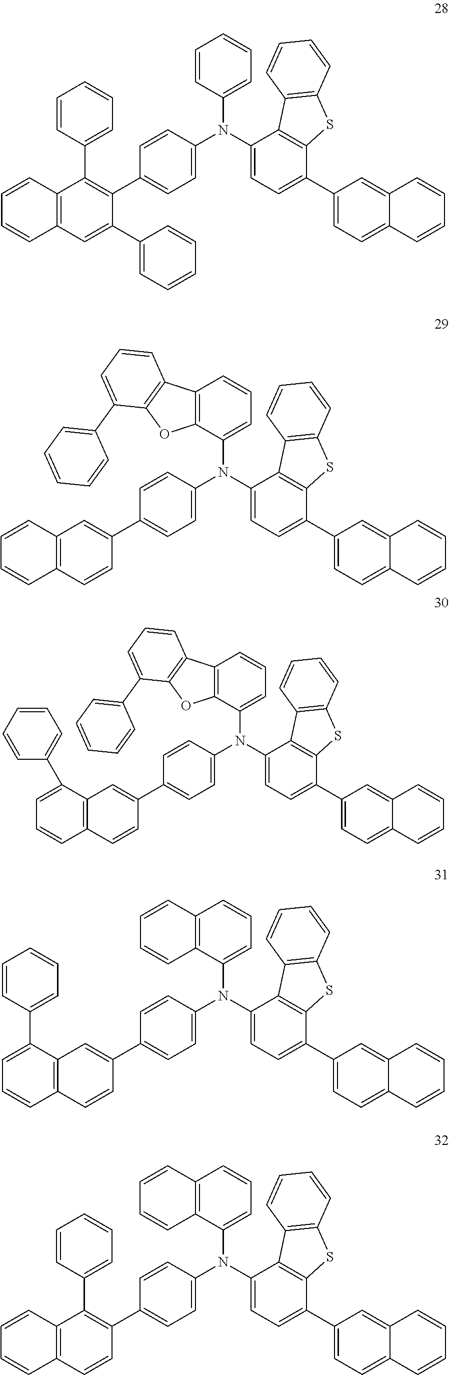 [SMILES: c1ccc(-c2cc3ccccc3c(-c3ccccc3)c2-c2ccc(N(c3ccccc3)c3ccc(-c4ccc5ccccc5c4)c4sc5ccccc5c34)cc2)cc1 ; c1ccc(-c2cccc3c2oc2c(N(c4ccc(-c5ccc6ccccc6c5)cc4)c4ccc(-c5ccc6ccccc6c5)c5sc6ccccc6c45)cccc23)cc1 ; c1ccc(-c2cccc3ccc(-c4ccc(N(c5cccc6c5oc5c(-c7ccccc7)cccc56)c5ccc(-c6ccc7ccccc7c6)c6sc7ccccc7c56)cc4)cc23)cc1 ; c1ccc(-c2cccc3ccc(-c4ccc(N(c5cccc6ccccc56)c5ccc(-c6ccc7ccccc7c6)c6sc7ccccc7c56)cc4)cc23)cc1 ; c1ccc(-c2c(-c3ccc(N(c4cccc5ccccc45)c4ccc(-c5ccc6ccccc6c5)c5sc6ccccc6c45)cc3)ccc3ccccc23)cc1] 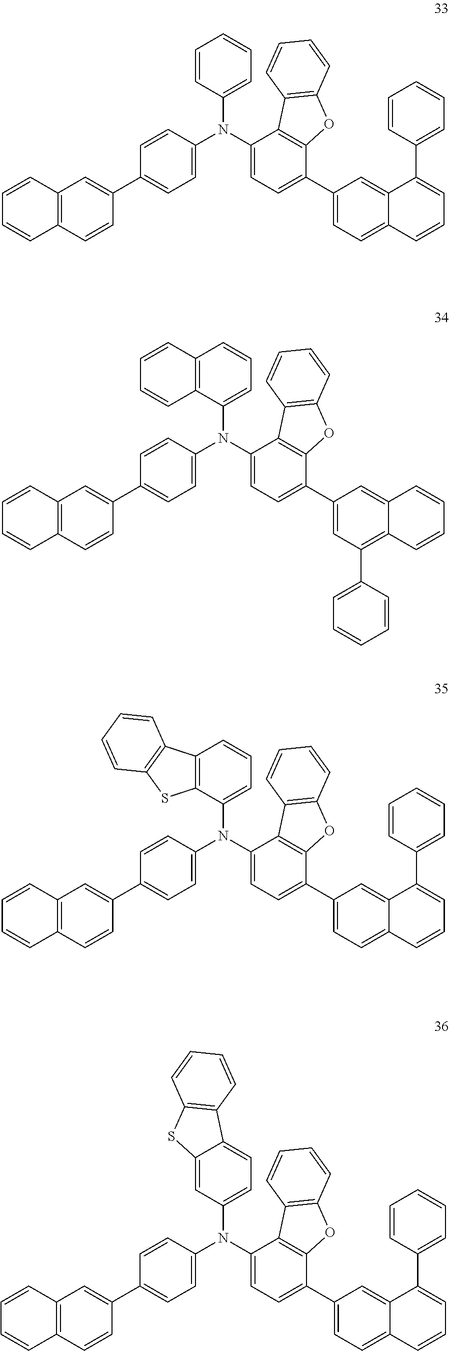 [SMILES: c1ccc(-c2cccc3ccc(-c4ccc(N(c5ccccc5)c5ccc(-c6ccc7ccccc7c6)cc5)c5c4oc4ccccc45)cc23)cc1 ; c1ccc(-c2cc(-c3ccc(N(c4ccc(-c5ccc6ccccc6c5)cc4)c4cccc5ccccc45)c4c3oc3ccccc34)cc3ccccc23)cc1 ; c1ccc(-c2cccc3ccc(-c4ccc(N(c5ccc(-c6ccc7ccccc7c6)cc5)c5cccc6c5sc5ccccc56)c5c4oc4ccccc45)cc23)cc1 ; c1ccc(-c2cccc3ccc(-c4ccc(N(c5ccc(-c6ccc7ccccc7c6)cc5)c5ccc6c(c5)sc5ccccc56)c5c4oc4ccccc45)cc23)cc1] 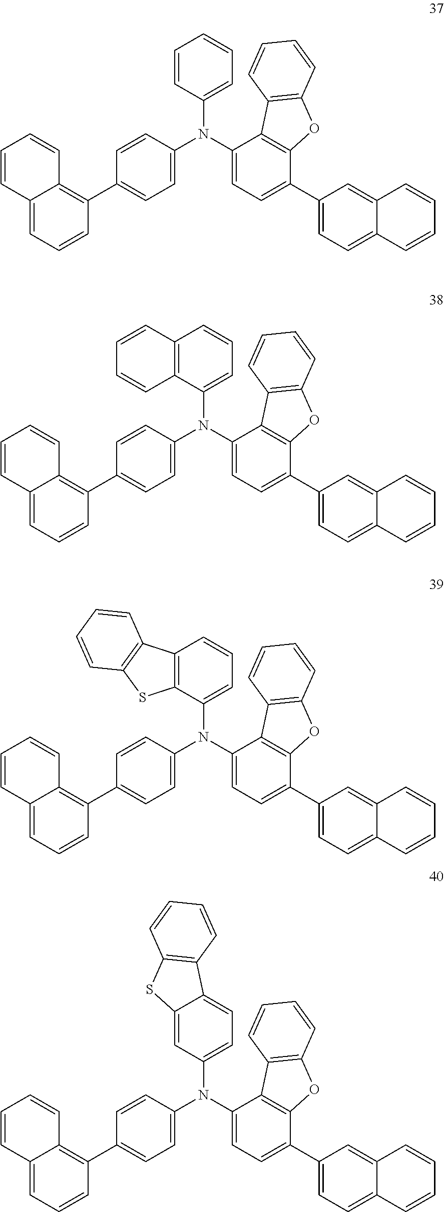 [SMILES: c1ccc(N(c2ccc(-c3cccc4ccccc34)cc2)c2ccc(-c3ccc4ccccc4c3)c3oc4ccccc4c23)cc1 ; c1ccc2cc(-c3ccc(N(c4ccc(-c5cccc6ccccc56)cc4)c4cccc5ccccc45)c4c3oc3ccccc34)ccc2c1 ; c1ccc2cc(-c3ccc(N(c4ccc(-c5cccc6ccccc56)cc4)c4cccc5c4sc4ccccc45)c4c3oc3ccccc34)ccc2c1 ; c1ccc2cc(-c3ccc(N(c4ccc(-c5cccc6ccccc56)cc4)c4ccc5c(c4)sc4ccccc45)c4c3oc3ccccc34)ccc2c1] 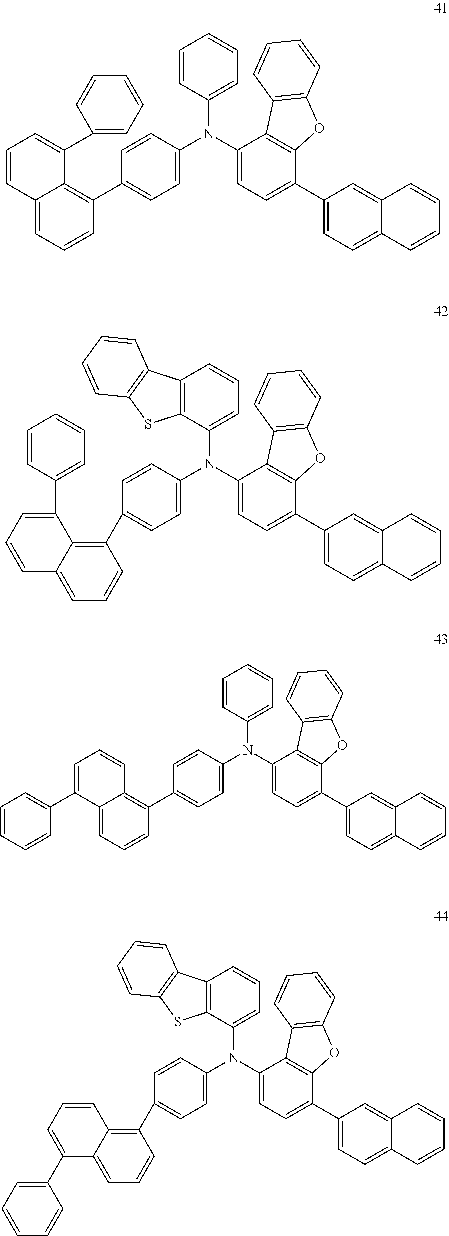 [SMILES: c1ccc(-c2cccc3cccc(-c4ccc(N(c5ccccc5)c5ccc(-c6ccc7ccccc7c6)c6oc7ccccc7c56)cc4)c23)cc1 ; c1ccc(-c2cccc3cccc(-c4ccc(N(c5cccc6c5sc5ccccc56)c5ccc(-c6ccc7ccccc7c6)c6oc7ccccc7c56)cc4)c23)cc1 ; c1ccc(-c2cccc3c(-c4ccc(N(c5ccccc5)c5ccc(-c6ccc7ccccc7c6)c6oc7ccccc7c56)cc4)cccc23)cc1 ; c1ccc(-c2cccc3c(-c4ccc(N(c5cccc6c5sc5ccccc56)c5ccc(-c6ccc7ccccc7c6)c6oc7ccccc7c56)cc4)cccc23)cc1] 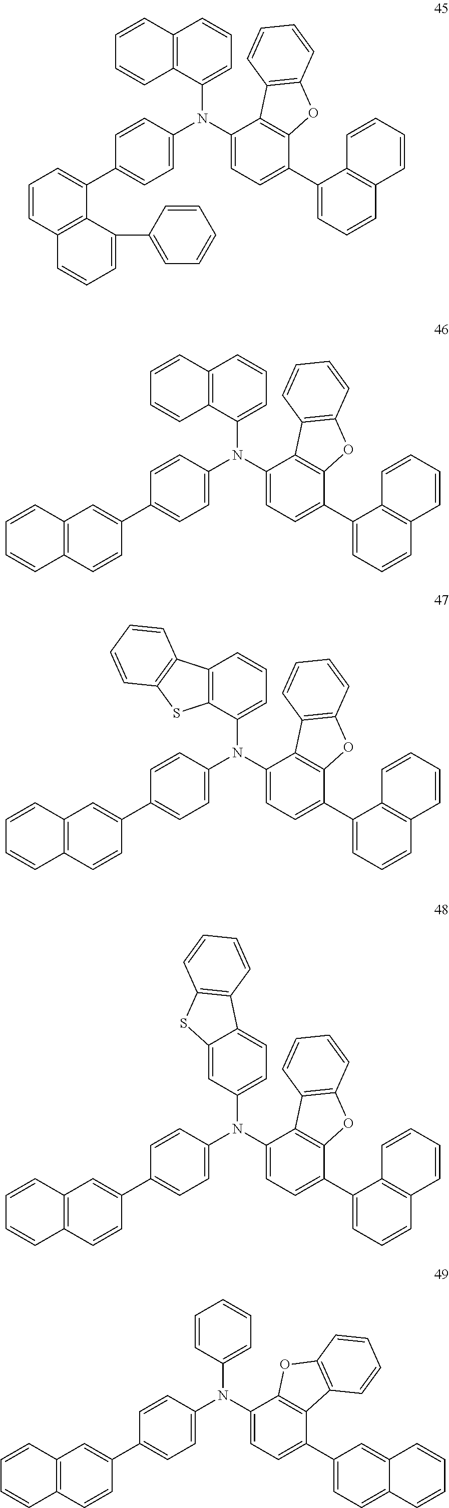 [SMILES: c1ccc(-c2cccc3cccc(-c4ccc(N(c5cccc6ccccc56)c5ccc(-c6cccc7ccccc67)c6oc7ccccc7c56)cc4)c23)cc1 ; c1ccc2cc(-c3ccc(N(c4cccc5ccccc45)c4ccc(-c5cccc6ccccc56)c5oc6ccccc6c45)cc3)ccc2c1 ; c1ccc2cc(-c3ccc(N(c4cccc5c4sc4ccccc45)c4ccc(-c5cccc6ccccc56)c5oc6ccccc6c45)cc3)ccc2c1 ; c1ccc2cc(-c3ccc(N(c4ccc5c(c4)sc4ccccc45)c4ccc(-c5cccc6ccccc56)c5oc6ccccc6c45)cc3)ccc2c1 ; c1ccc(N(c2ccc(-c3ccc4ccccc4c3)cc2)c2ccc(-c3ccc4ccccc4c3)c3c2oc2ccccc23)cc1] 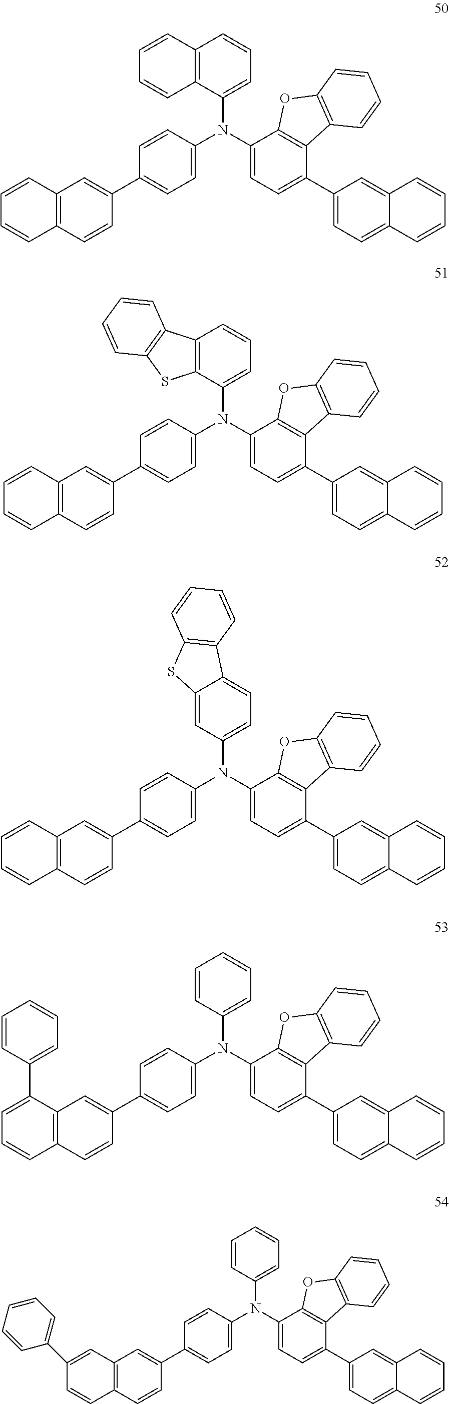 [SMILES: c1ccc2cc(-c3ccc(N(c4cccc5ccccc45)c4ccc(-c5ccc6ccccc6c5)c5c4oc4ccccc45)cc3)ccc2c1 ; c1ccc2cc(-c3ccc(N(c4ccc(-c5ccc6ccccc6c5)c5c4oc4ccccc45)c4cccc5c4sc4ccccc45)cc3)ccc2c1 ; c1ccc2cc(-c3ccc(N(c4ccc5c(c4)sc4ccccc45)c4ccc(-c5ccc6ccccc6c5)c5c4oc4ccccc45)cc3)ccc2c1 ; c1ccc(-c2cccc3ccc(-c4ccc(N(c5ccccc5)c5ccc(-c6ccc7ccccc7c6)c6c5oc5ccccc56)cc4)cc23)cc1 ; c1ccc(-c2ccc3ccc(-c4ccc(N(c5ccccc5)c5ccc(-c6ccc7ccccc7c6)c6c5oc5ccccc56)cc4)cc3c2)cc1] 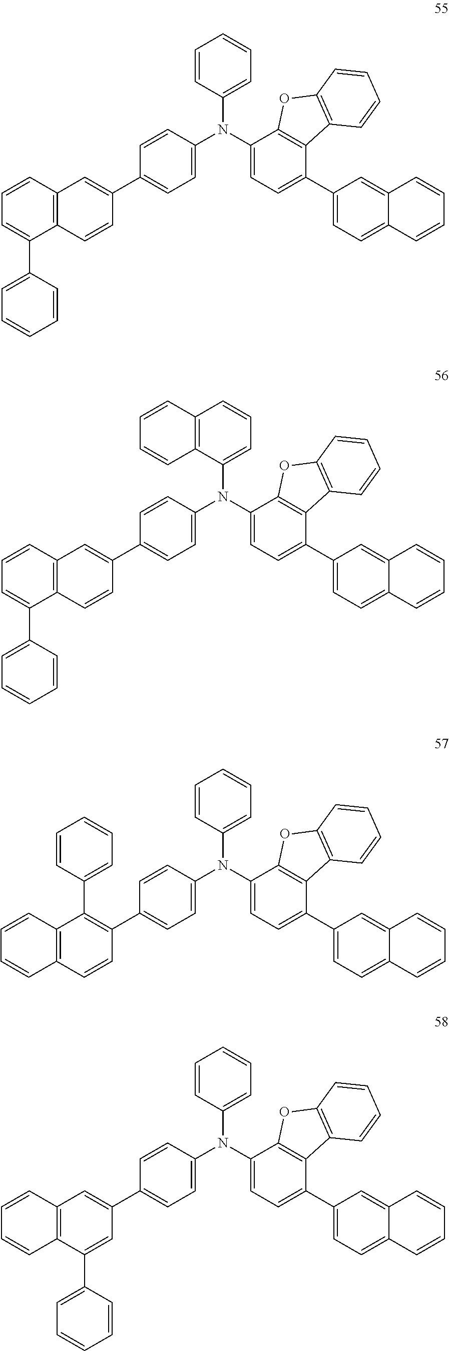 [SMILES: c1ccc(-c2cccc3cc(-c4ccc(N(c5ccccc5)c5ccc(-c6ccc7ccccc7c6)c6c5oc5ccccc56)cc4)ccc23)cc1 ; c1ccc(-c2cccc3cc(-c4ccc(N(c5cccc6ccccc56)c5ccc(-c6ccc7ccccc7c6)c6c5oc5ccccc56)cc4)ccc23)cc1 ; c1ccc(-c2c(-c3ccc(N(c4ccccc4)c4ccc(-c5ccc6ccccc6c5)c5c4oc4ccccc45)cc3)ccc3ccccc23)cc1 ; c1ccc(-c2cc(-c3ccc(N(c4ccccc4)c4ccc(-c5ccc6ccccc6c5)c5c4oc4ccccc45)cc3)cc3ccccc23)cc1] 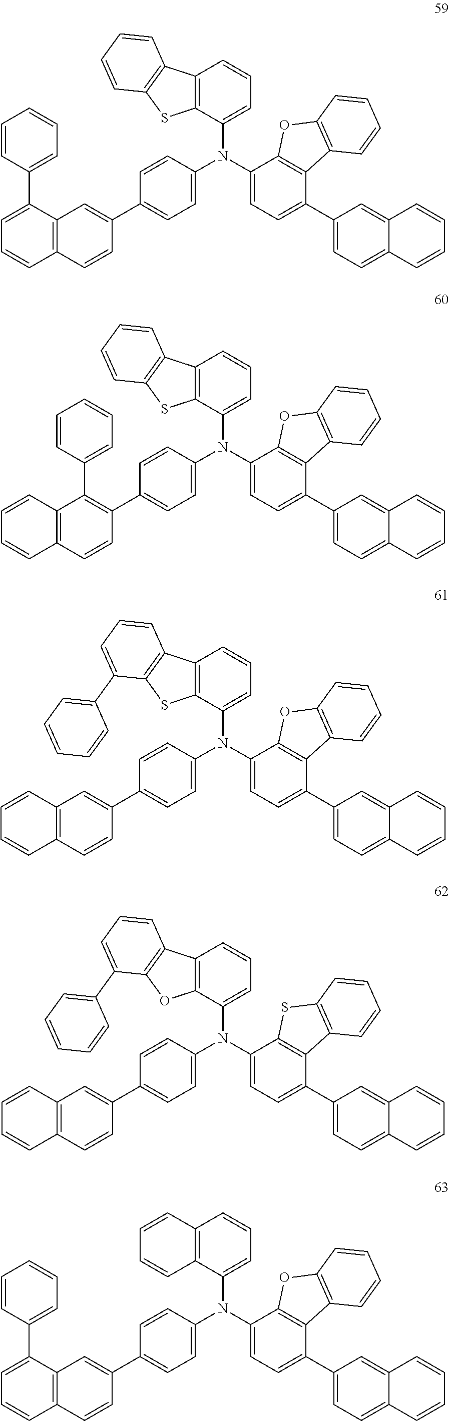 [SMILES: c1ccc(-c2cccc3ccc(-c4ccc(N(c5ccc(-c6ccc7ccccc7c6)c6c5oc5ccccc56)c5cccc6c5sc5ccccc56)cc4)cc23)cc1 ; c1ccc(-c2c(-c3ccc(N(c4ccc(-c5ccc6ccccc6c5)c5c4oc4ccccc45)c4cccc5c4sc4ccccc45)cc3)ccc3ccccc23)cc1 ; c1ccc(-c2cccc3c2sc2c(N(c4ccc(-c5ccc6ccccc6c5)cc4)c4ccc(-c5ccc6ccccc6c5)c5c4oc4ccccc45)cccc23)cc1 ; c1ccc(-c2cccc3c2oc2c(N(c4ccc(-c5ccc6ccccc6c5)cc4)c4ccc(-c5ccc6ccccc6c5)c5c4sc4ccccc45)cccc23)cc1 ; c1ccc(-c2cccc3ccc(-c4ccc(N(c5cccc6ccccc56)c5ccc(-c6ccc7ccccc7c6)c6c5oc5ccccc56)cc4)cc23)cc1] 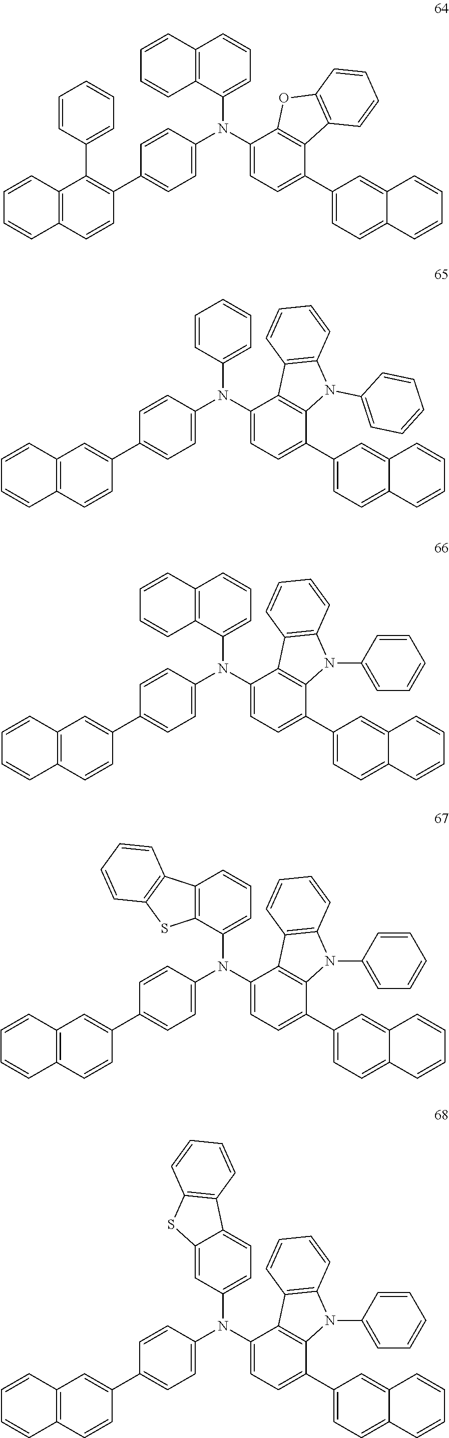 [SMILES: c1ccc(-c2c(-c3ccc(N(c4cccc5ccccc45)c4ccc(-c5ccc6ccccc6c5)c5c4oc4ccccc45)cc3)ccc3ccccc23)cc1 ; c1ccc(N(c2ccc(-c3ccc4ccccc4c3)cc2)c2ccc(-c3ccc4ccccc4c3)c3c2c2ccccc2n3-c2ccccc2)cc1 ; c1ccc(-n2c3ccccc3c3c(N(c4ccc(-c5ccc6ccccc6c5)cc4)c4cccc5ccccc45)ccc(-c4ccc5ccccc5c4)c32)cc1 ; c1ccc(-n2c3ccccc3c3c(N(c4ccc(-c5ccc6ccccc6c5)cc4)c4cccc5c4sc4ccccc45)ccc(-c4ccc5ccccc5c4)c32)cc1 ; c1ccc(-n2c3ccccc3c3c(N(c4ccc(-c5ccc6ccccc6c5)cc4)c4ccc5c(c4)sc4ccccc45)ccc(-c4ccc5ccccc5c4)c32)cc1] 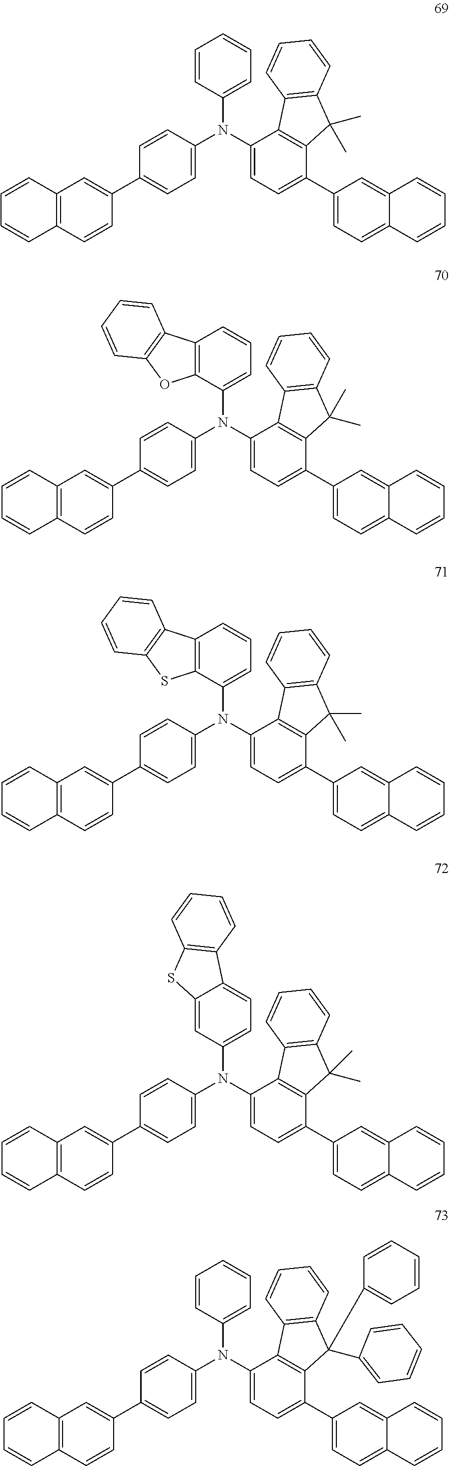 [SMILES: CC1(C)c2ccccc2-c2c(N(c3ccccc3)c3ccc(-c4ccc5ccccc5c4)cc3)ccc(-c3ccc4ccccc4c3)c21 ; CC1(C)c2ccccc2-c2c(N(c3ccc(-c4ccc5ccccc5c4)cc3)c3cccc4c3oc3ccccc34)ccc(-c3ccc4ccccc4c3)c21 ; CC1(C)c2ccccc2-c2c(N(c3ccc(-c4ccc5ccccc5c4)cc3)c3cccc4c3sc3ccccc34)ccc(-c3ccc4ccccc4c3)c21 ; CC1(C)c2ccccc2-c2c(N(c3ccc(-c4ccc5ccccc5c4)cc3)c3ccc4c(c3)sc3ccccc34)ccc(-c3ccc4ccccc4c3)c21 ; c1ccc(N(c2ccc(-c3ccc4ccccc4c3)cc2)c2ccc(-c3ccc4ccccc4c3)c3c2-c2ccccc2C3(c2ccccc2)c2ccccc2)cc1] 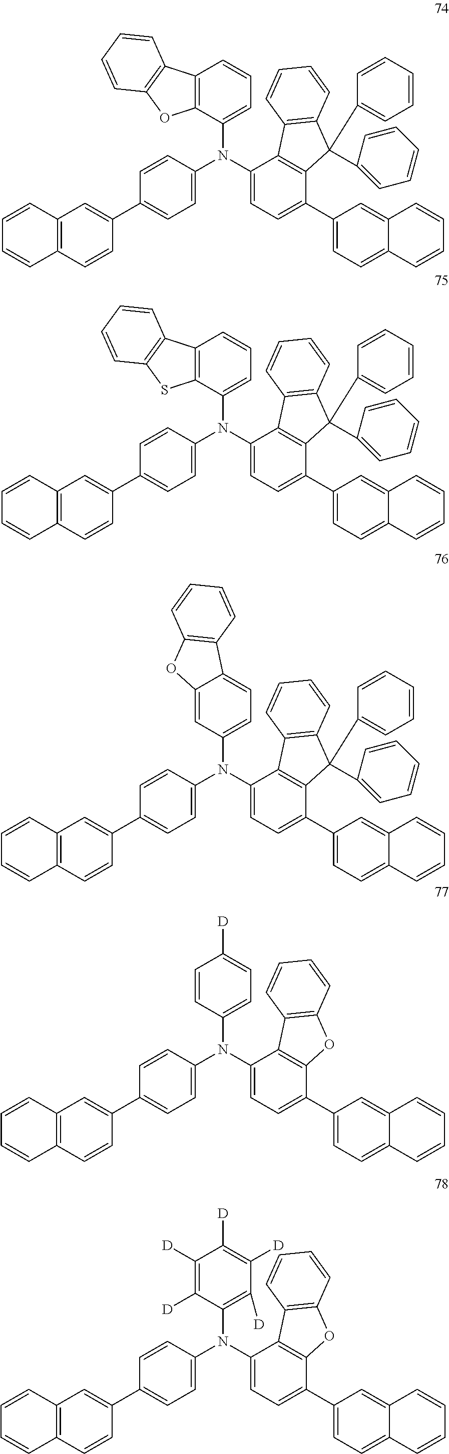 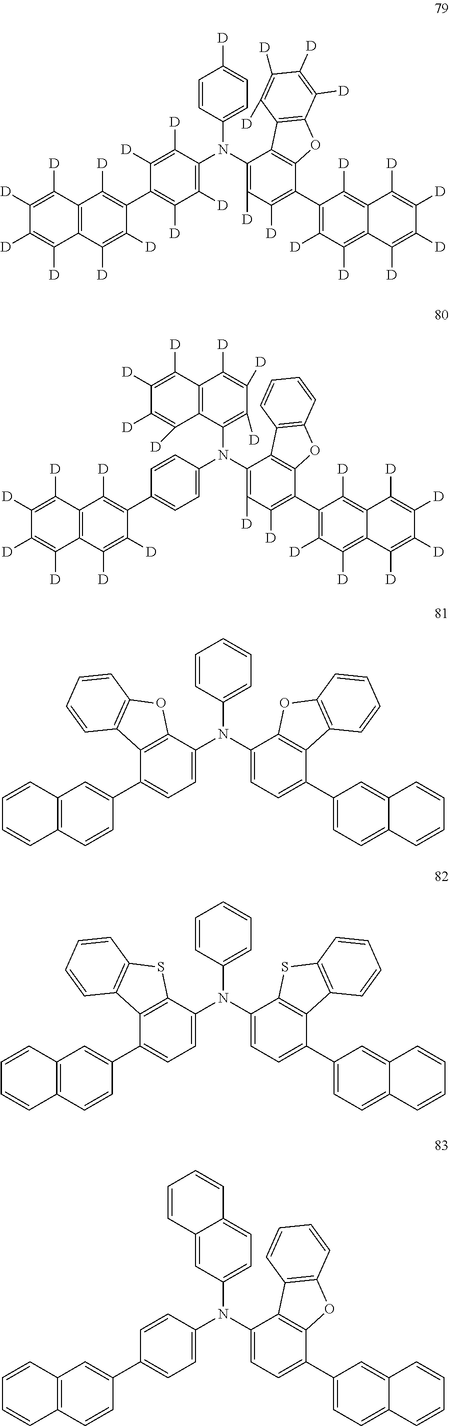 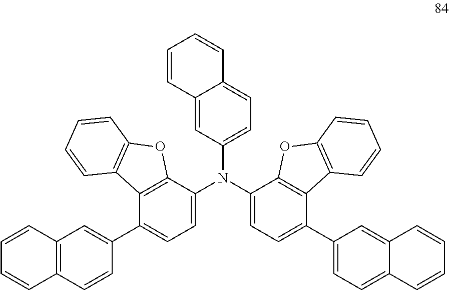 [SMILES: c1ccc2cc(-c3ccc(N(c4ccc5ccccc5c4)c4ccc(-c5ccc6ccccc6c5)c5c4oc4ccccc45)c4oc5ccccc5c34)ccc2c1]
wherein in Compound Group 1,
D indicates a deuterium atom.

13. An amine compound represented by Formula 1: 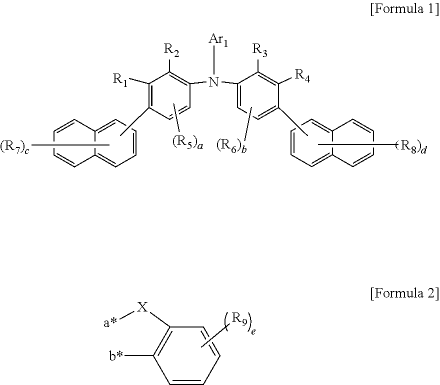
wherein in Formula 1,
Ar₁ is a substituted or unsubstituted aryl group having 6 to 40 ring-forming carbon atoms other than a phenanthryl group, or a substituted or unsubstituted heteroaryl group having 5 to 40 ring-forming carbon atoms,
R₁ to R₄ are each independently a hydrogen atom, a deuterium atom, a substituted or unsubstituted alkyl group having 1 to 20 carbon atoms, a substituted or unsubstituted aryl group having 6 to 40 ring-forming carbon atoms, or a single bond forming a ring by bonding with a group represented by Formula 2, and
a pair of R₁ and R₂ and/or a pair of R₃ and R₄ are bonded with a group represented by Formula 2 to form a ring,
wherein in Formula 2,
X is O, S, N(R₁₀), or C(R₁₁)(R₁₂), and
a* and b* each indicate a bond to one of R₁ to R₄ in Formula 1, and
wherein in Formula 1 and Formula 2,
R₅ to R₁₂ are each independently a hydrogen atom, a deuterium atom, a substituted or unsubstituted alkyl group having 1 to 20 carbon atoms, or a substituted or unsubstituted aryl group having 6 to 40 ring-forming carbon atoms,
a and b are each independently an integer from 0 to 2,
c and d are each independently an integer from 0 to 7, and
e is an integer from 0 to 4.

14. The amine compound of claim 13, wherein the amine compound represented by Formula 1 is represented by one of Formulas 1-1 to 1-3: 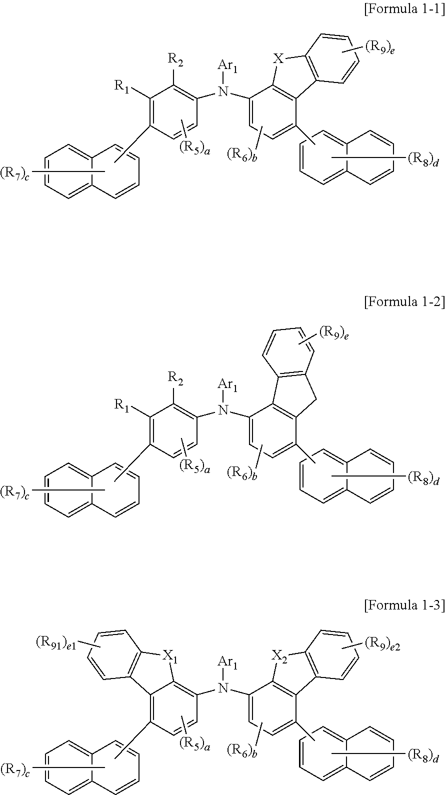
wherein in Formula 1-3,
X₁ and X₂ are each independently O, S, N(R₁₀), or C(R₁₁)(R₁₂),
e1 and e2 are each independently an integer from 0 to 4, and
R₉₁ and R₉₂ are each independently a hydrogen atom, a deuterium atom, a substituted or unsubstituted alkyl group having 1 to 20 carbon atoms, or a substituted or unsubstituted aryl group having 6 to 40 ring-forming carbon atoms, and
wherein in Formulas 1-1 to 1-3,
Ar₁, X, R₁ to R₁₂, and a to e are the same as defined in Formulas 1 and 2.

15. The amine compound of claim 14, wherein in Formula 1-3,
X₁ and X₂ are the same.

16. The amine compound of claim 13, wherein the group represented by Formula 2 is represented by one of Formulas 2-1 to 2-5: 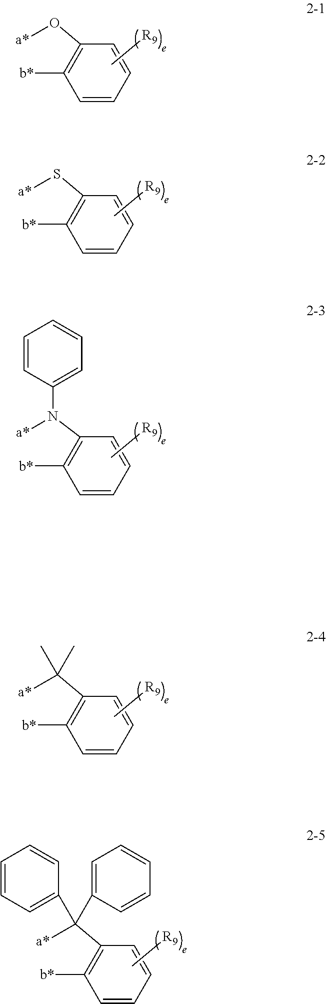
wherein in Formulas 2-1 to 2-5,
R₉, e, a*, and b* are the same as defined in Formula 2.

17. The amine compound of claim 13, wherein in Formula 1,
Ar₁ is a substituted or unsubstituted phenyl group, a substituted or unsubstituted naphthyl group, a substituted or unsubstituted dibenzothiophene group, or a substituted or unsubstituted dibenzofuran group.

18. The amine compound of claim 13, wherein in Formula 1,
at least one of R₁ to R₉ and Ar₁ is a deuterium atom or a substituent including a deuterium atom.

19. The amine compound of claim 13, wherein the amine compound represented by Formula 1 is a monoamine compound.

20. The amine compound of claim 13, wherein the amine compound is selected from Compound Group 1: 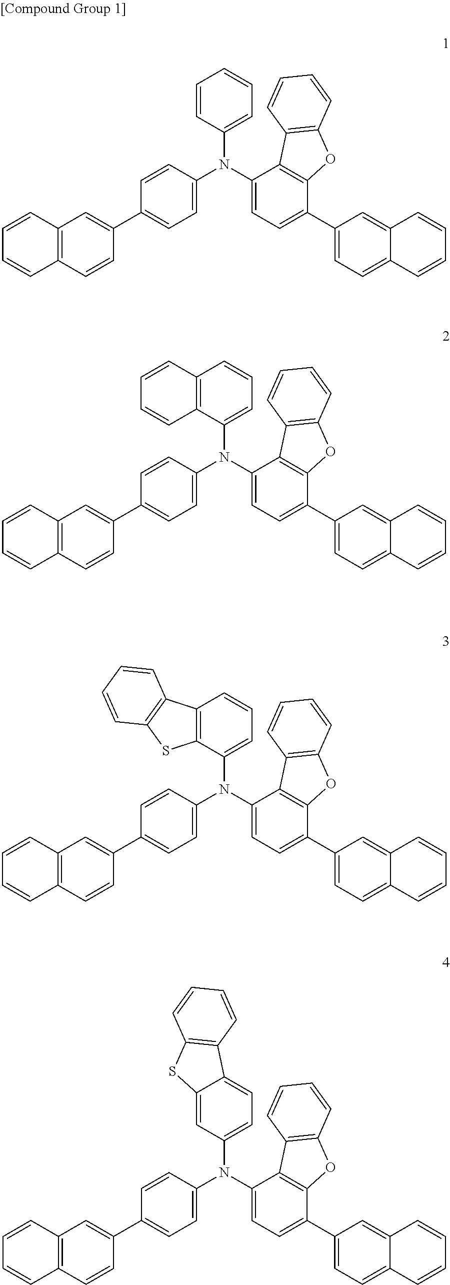 [SMILES: c1ccc(N(c2ccc(-c3ccc4ccccc4c3)cc2)c2ccc(-c3ccc4ccccc4c3)c3oc4ccccc4c23)cc1 ; c1ccc2cc(-c3ccc(N(c4cccc5ccccc45)c4ccc(-c5ccc6ccccc6c5)c5oc6ccccc6c45)cc3)ccc2c1 ; c1ccc2cc(-c3ccc(N(c4cccc5c4sc4ccccc45)c4ccc(-c5ccc6ccccc6c5)c5oc6ccccc6c45)cc3)ccc2c1 ; c1ccc2cc(-c3ccc(N(c4ccc5c(c4)sc4ccccc45)c4ccc(-c5ccc6ccccc6c5)c5oc6ccccc6c45)cc3)ccc2c1] 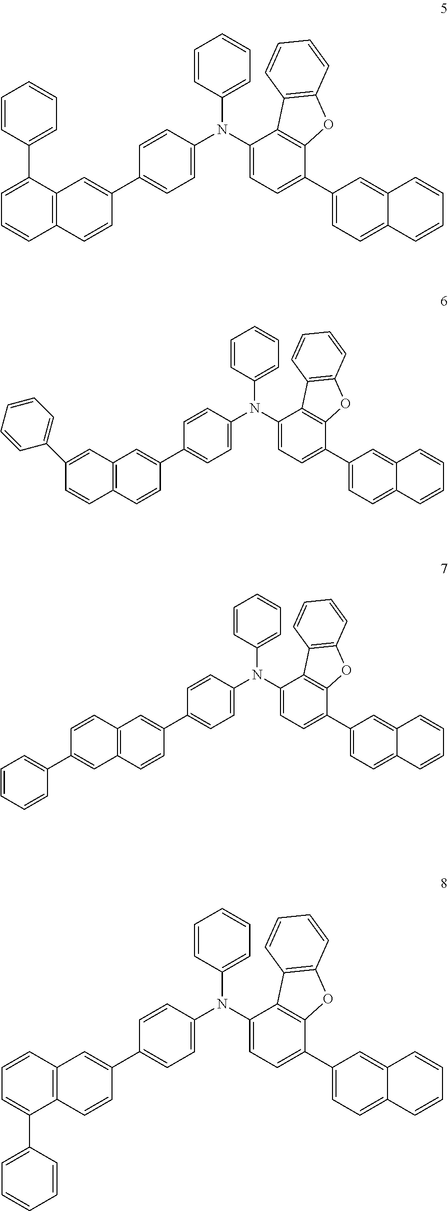 [SMILES: c1ccc(-c2cccc3ccc(-c4ccc(N(c5ccccc5)c5ccc(-c6ccc7ccccc7c6)c6oc7ccccc7c56)cc4)cc23)cc1 ; c1ccc(-c2ccc3ccc(-c4ccc(N(c5ccccc5)c5ccc(-c6ccc7ccccc7c6)c6oc7ccccc7c56)cc4)cc3c2)cc1 ; c1ccc(-c2ccc3cc(-c4ccc(N(c5ccccc5)c5ccc(-c6ccc7ccccc7c6)c6oc7ccccc7c56)cc4)ccc3c2)cc1 ; c1ccc(-c2cccc3cc(-c4ccc(N(c5ccccc5)c5ccc(-c6ccc7ccccc7c6)c6oc7ccccc7c56)cc4)ccc23)cc1] 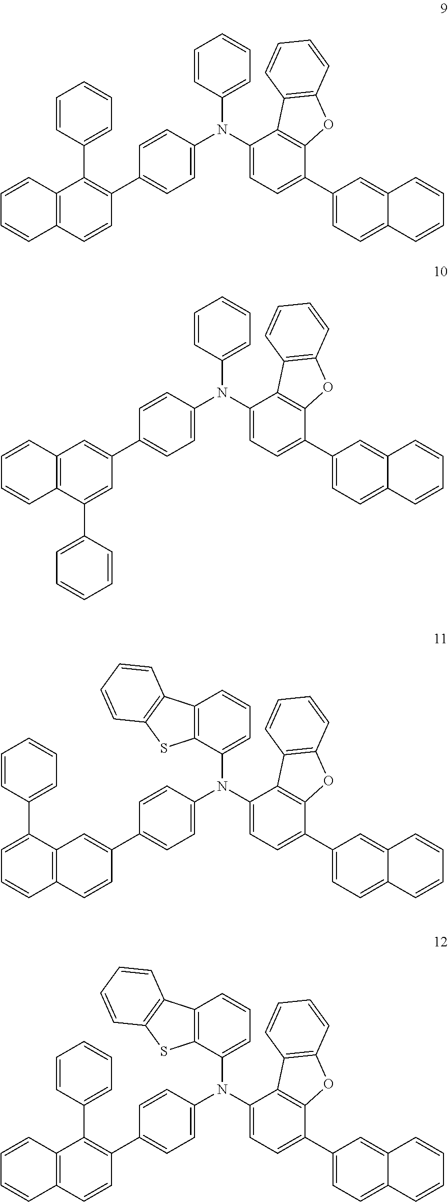 [SMILES: c1ccc(-c2c(-c3ccc(N(c4ccccc4)c4ccc(-c5ccc6ccccc6c5)c5oc6ccccc6c45)cc3)ccc3ccccc23)cc1 ; c1ccc(-c2cc(-c3ccc(N(c4ccccc4)c4ccc(-c5ccc6ccccc6c5)c5oc6ccccc6c45)cc3)cc3ccccc23)cc1 ; c1ccc(-c2cccc3ccc(-c4ccc(N(c5cccc6c5sc5ccccc56)c5ccc(-c6ccc7ccccc7c6)c6oc7ccccc7c56)cc4)cc23)cc1 ; c1ccc(-c2c(-c3ccc(N(c4cccc5c4sc4ccccc45)c4ccc(-c5ccc6ccccc6c5)c5oc6ccccc6c45)cc3)ccc3ccccc23)cc1] 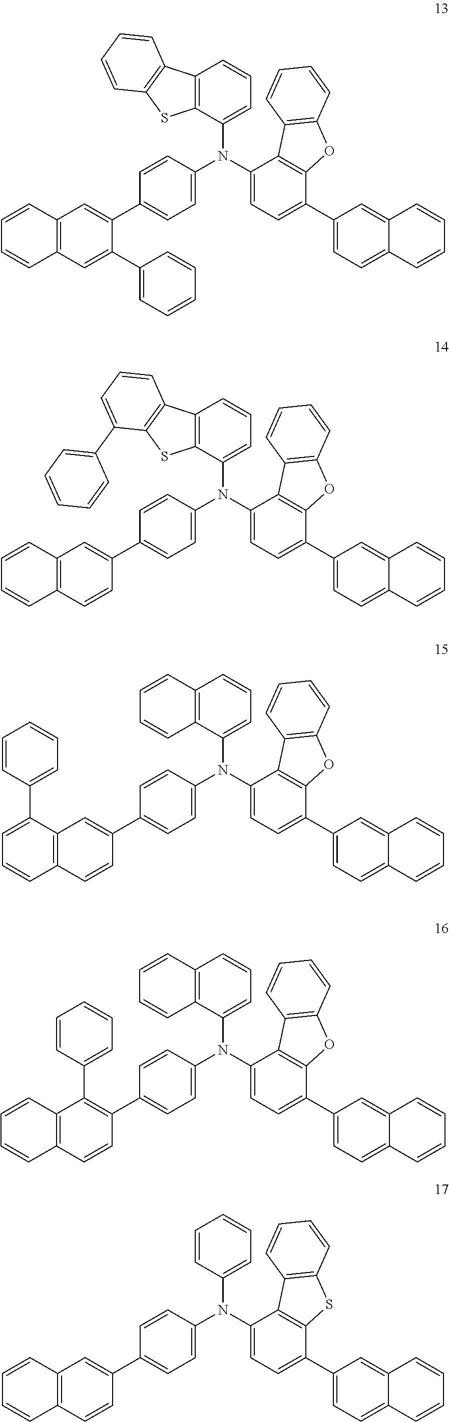 [SMILES: c1ccc(-c2cc3ccccc3cc2-c2ccc(N(c3cccc4c3sc3ccccc34)c3ccc(-c4ccc5ccccc5c4)c4oc5ccccc5c34)cc2)cc1 ; c1ccc(-c2cccc3c2sc2c(N(c4ccc(-c5ccc6ccccc6c5)cc4)c4ccc(-c5ccc6ccccc6c5)c5oc6ccccc6c45)cccc23)cc1 ; c1ccc(-c2cccc3ccc(-c4ccc(N(c5cccc6ccccc56)c5ccc(-c6ccc7ccccc7c6)c6oc7ccccc7c56)cc4)cc23)cc1 ; c1ccc(-c2c(-c3ccc(N(c4cccc5ccccc45)c4ccc(-c5ccc6ccccc6c5)c5oc6ccccc6c45)cc3)ccc3ccccc23)cc1 ; c1ccc(N(c2ccc(-c3ccc4ccccc4c3)cc2)c2ccc(-c3ccc4ccccc4c3)c3sc4ccccc4c23)cc1] 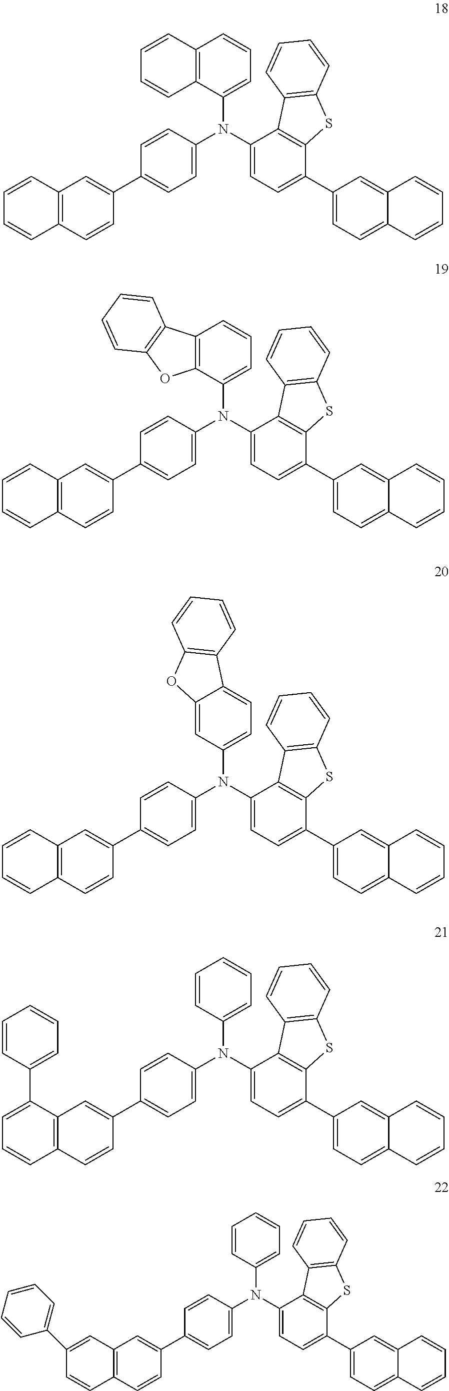 [SMILES: c1ccc2cc(-c3ccc(N(c4cccc5ccccc45)c4ccc(-c5ccc6ccccc6c5)c5sc6ccccc6c45)cc3)ccc2c1 ; c1ccc2cc(-c3ccc(N(c4cccc5c4oc4ccccc45)c4ccc(-c5ccc6ccccc6c5)c5sc6ccccc6c45)cc3)ccc2c1 ; c1ccc2cc(-c3ccc(N(c4ccc5c(c4)oc4ccccc45)c4ccc(-c5ccc6ccccc6c5)c5sc6ccccc6c45)cc3)ccc2c1 ; c1ccc(-c2cccc3ccc(-c4ccc(N(c5ccccc5)c5ccc(-c6ccc7ccccc7c6)c6sc7ccccc7c56)cc4)cc23)cc1 ; c1ccc(-c2ccc3ccc(-c4ccc(N(c5ccccc5)c5ccc(-c6ccc7ccccc7c6)c6sc7ccccc7c56)cc4)cc3c2)cc1] 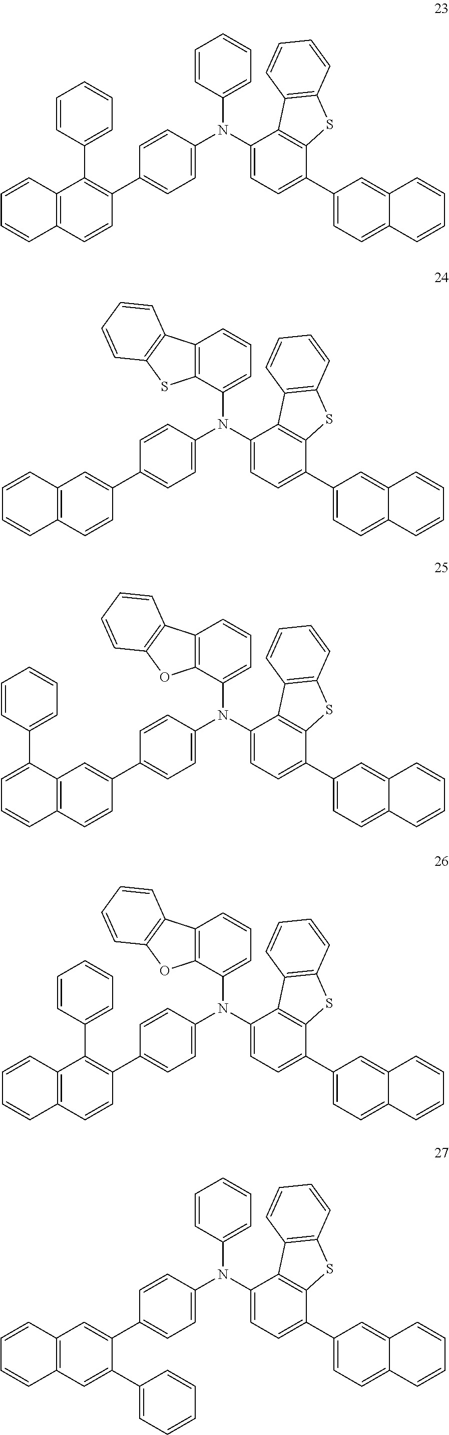 [SMILES: c1ccc(-c2c(-c3ccc(N(c4ccccc4)c4ccc(-c5ccc6ccccc6c5)c5sc6ccccc6c45)cc3)ccc3ccccc23)cc1 ; c1ccc2cc(-c3ccc(N(c4cccc5c4sc4ccccc45)c4ccc(-c5ccc6ccccc6c5)c5sc6ccccc6c45)cc3)ccc2c1 ; c1ccc(-c2cccc3ccc(-c4ccc(N(c5cccc6c5oc5ccccc56)c5ccc(-c6ccc7ccccc7c6)c6sc7ccccc7c56)cc4)cc23)cc1 ; c1ccc(-c2c(-c3ccc(N(c4cccc5c4oc4ccccc45)c4ccc(-c5ccc6ccccc6c5)c5sc6ccccc6c45)cc3)ccc3ccccc23)cc1 ; c1ccc(-c2cc3ccccc3cc2-c2ccc(N(c3ccccc3)c3ccc(-c4ccc5ccccc5c4)c4sc5ccccc5c34)cc2)cc1] 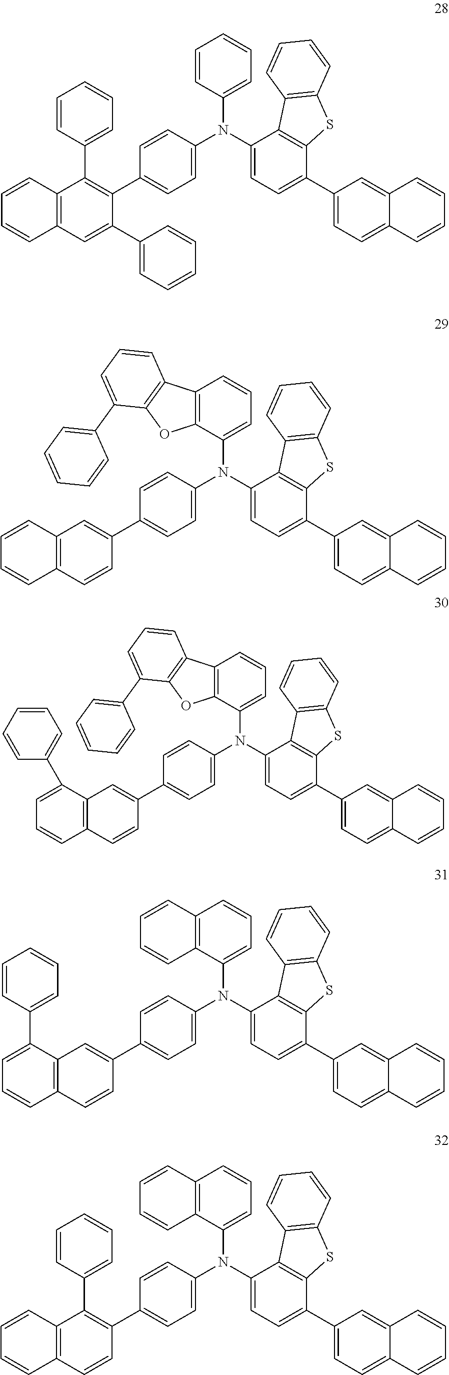 [SMILES: c1ccc(-c2cc3ccccc3c(-c3ccccc3)c2-c2ccc(N(c3ccccc3)c3ccc(-c4ccc5ccccc5c4)c4sc5ccccc5c34)cc2)cc1 ; c1ccc(-c2cccc3c2oc2c(N(c4ccc(-c5ccc6ccccc6c5)cc4)c4ccc(-c5ccc6ccccc6c5)c5sc6ccccc6c45)cccc23)cc1 ; c1ccc(-c2cccc3ccc(-c4ccc(N(c5cccc6c5oc5c(-c7ccccc7)cccc56)c5ccc(-c6ccc7ccccc7c6)c6sc7ccccc7c56)cc4)cc23)cc1 ; c1ccc(-c2cccc3ccc(-c4ccc(N(c5cccc6ccccc56)c5ccc(-c6ccc7ccccc7c6)c6sc7ccccc7c56)cc4)cc23)cc1 ; c1ccc(-c2c(-c3ccc(N(c4cccc5ccccc45)c4ccc(-c5ccc6ccccc6c5)c5sc6ccccc6c45)cc3)ccc3ccccc23)cc1] 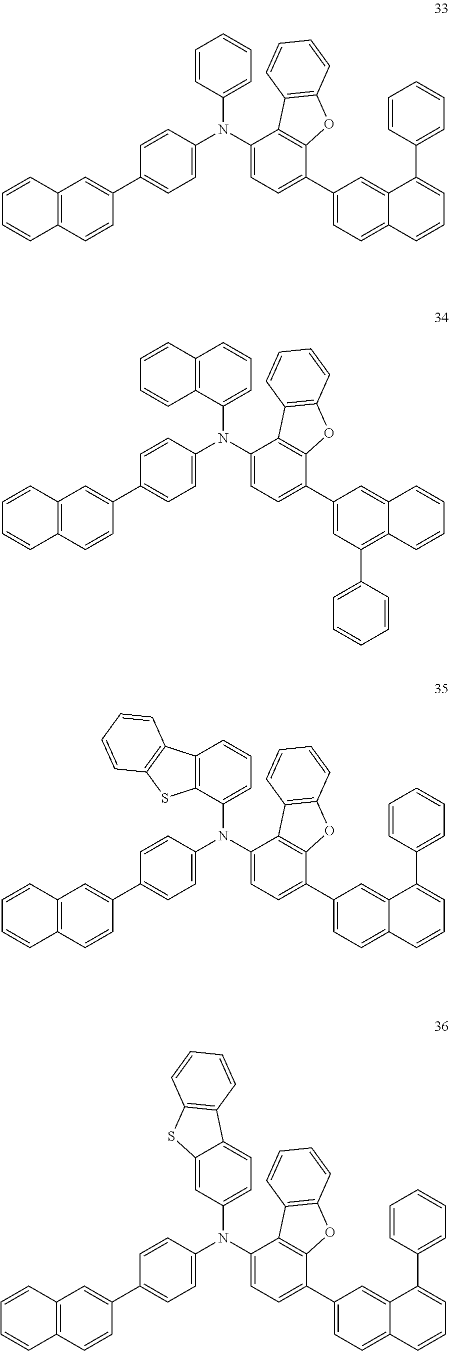 [SMILES: c1ccc(-c2cccc3ccc(-c4ccc(N(c5ccccc5)c5ccc(-c6ccc7ccccc7c6)cc5)c5c4oc4ccccc45)cc23)cc1 ; c1ccc(-c2cc(-c3ccc(N(c4ccc(-c5ccc6ccccc6c5)cc4)c4cccc5ccccc45)c4c3oc3ccccc34)cc3ccccc23)cc1 ; c1ccc(-c2cccc3ccc(-c4ccc(N(c5ccc(-c6ccc7ccccc7c6)cc5)c5cccc6c5sc5ccccc56)c5c4oc4ccccc45)cc23)cc1 ; c1ccc(-c2cccc3ccc(-c4ccc(N(c5ccc(-c6ccc7ccccc7c6)cc5)c5ccc6c(c5)sc5ccccc56)c5c4oc4ccccc45)cc23)cc1] 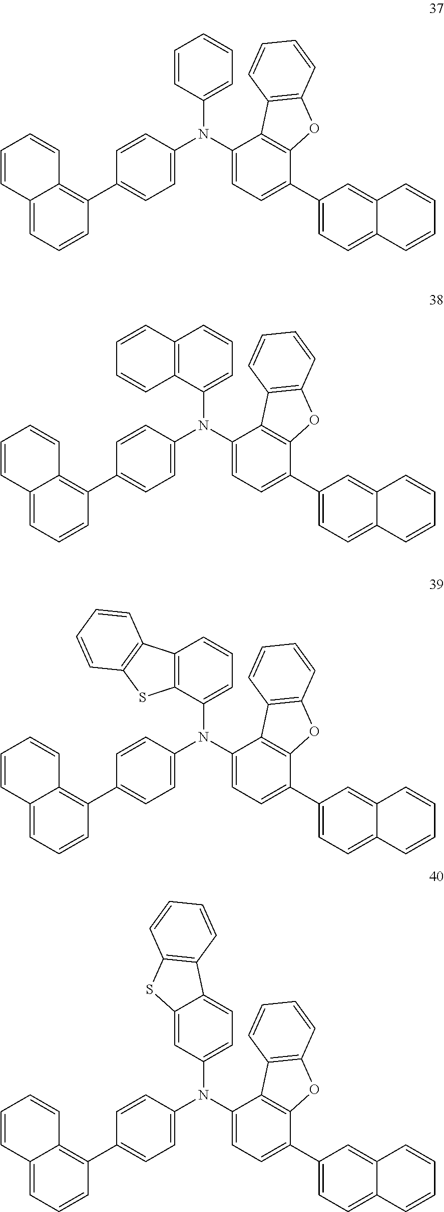 [SMILES: c1ccc(N(c2ccc(-c3cccc4ccccc34)cc2)c2ccc(-c3ccc4ccccc4c3)c3oc4ccccc4c23)cc1 ; c1ccc2cc(-c3ccc(N(c4ccc(-c5cccc6ccccc56)cc4)c4cccc5ccccc45)c4c3oc3ccccc34)ccc2c1 ; c1ccc2cc(-c3ccc(N(c4ccc(-c5cccc6ccccc56)cc4)c4cccc5c4sc4ccccc45)c4c3oc3ccccc34)ccc2c1 ; c1ccc2cc(-c3ccc(N(c4ccc(-c5cccc6ccccc56)cc4)c4ccc5c(c4)sc4ccccc45)c4c3oc3ccccc34)ccc2c1] 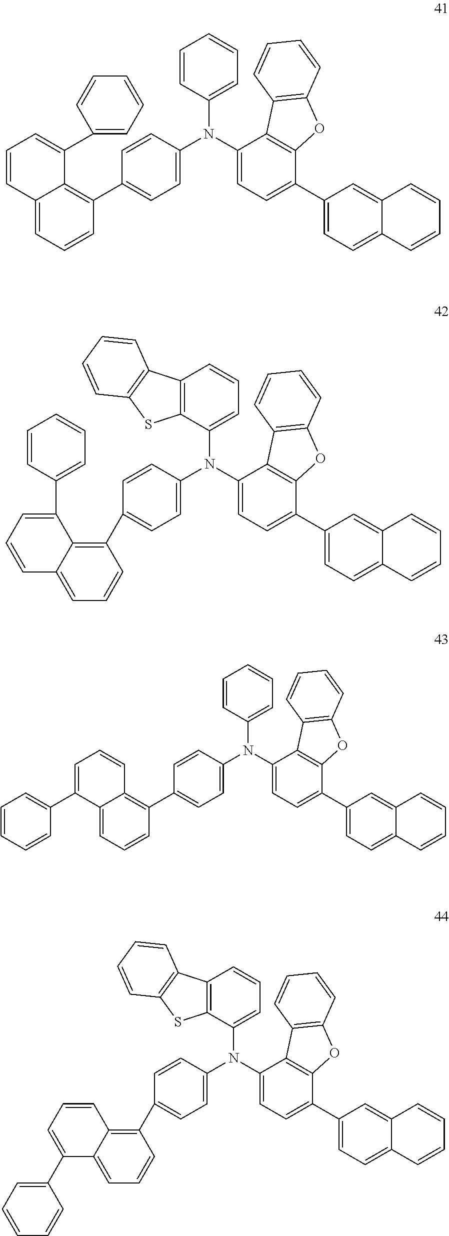 [SMILES: c1ccc(-c2cccc3cccc(-c4ccc(N(c5ccccc5)c5ccc(-c6ccc7ccccc7c6)c6oc7ccccc7c56)cc4)c23)cc1 ; c1ccc(-c2cccc3cccc(-c4ccc(N(c5cccc6c5sc5ccccc56)c5ccc(-c6ccc7ccccc7c6)c6oc7ccccc7c56)cc4)c23)cc1 ; c1ccc(-c2cccc3c(-c4ccc(N(c5ccccc5)c5ccc(-c6ccc7ccccc7c6)c6oc7ccccc7c56)cc4)cccc23)cc1 ; c1ccc(-c2cccc3c(-c4ccc(N(c5cccc6c5sc5ccccc56)c5ccc(-c6ccc7ccccc7c6)c6oc7ccccc7c56)cc4)cccc23)cc1] 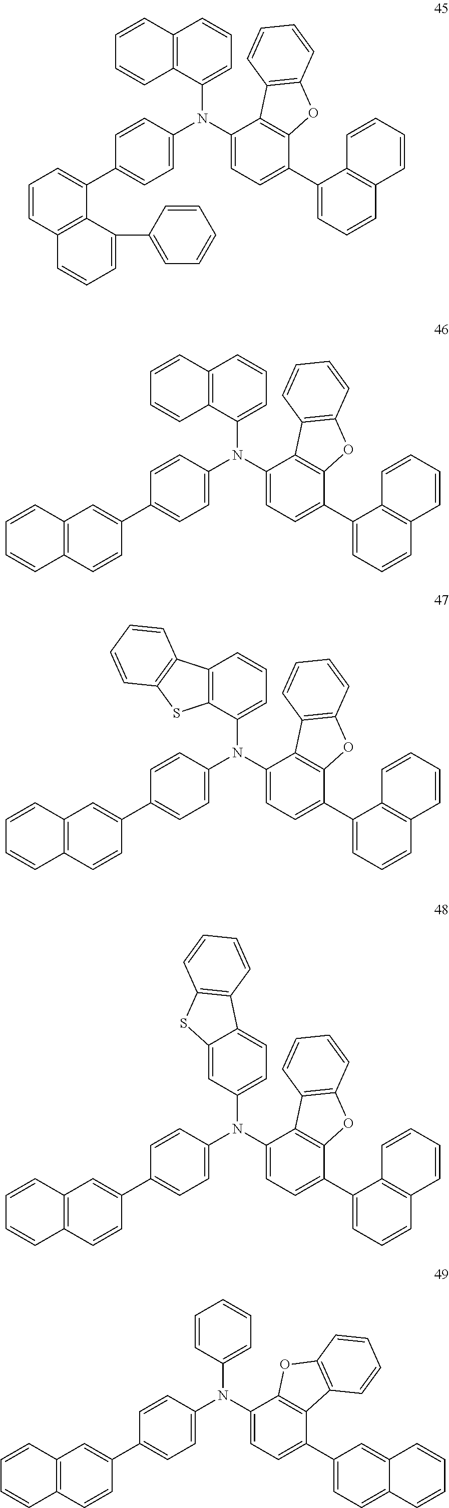 [SMILES: c1ccc(-c2cccc3cccc(-c4ccc(N(c5cccc6ccccc56)c5ccc(-c6cccc7ccccc67)c6oc7ccccc7c56)cc4)c23)cc1 ; c1ccc2cc(-c3ccc(N(c4cccc5ccccc45)c4ccc(-c5cccc6ccccc56)c5oc6ccccc6c45)cc3)ccc2c1 ; c1ccc2cc(-c3ccc(N(c4cccc5c4sc4ccccc45)c4ccc(-c5cccc6ccccc56)c5oc6ccccc6c45)cc3)ccc2c1 ; c1ccc2cc(-c3ccc(N(c4ccc5c(c4)sc4ccccc45)c4ccc(-c5cccc6ccccc56)c5oc6ccccc6c45)cc3)ccc2c1 ; c1ccc(N(c2ccc(-c3ccc4ccccc4c3)cc2)c2ccc(-c3ccc4ccccc4c3)c3c2oc2ccccc23)cc1] 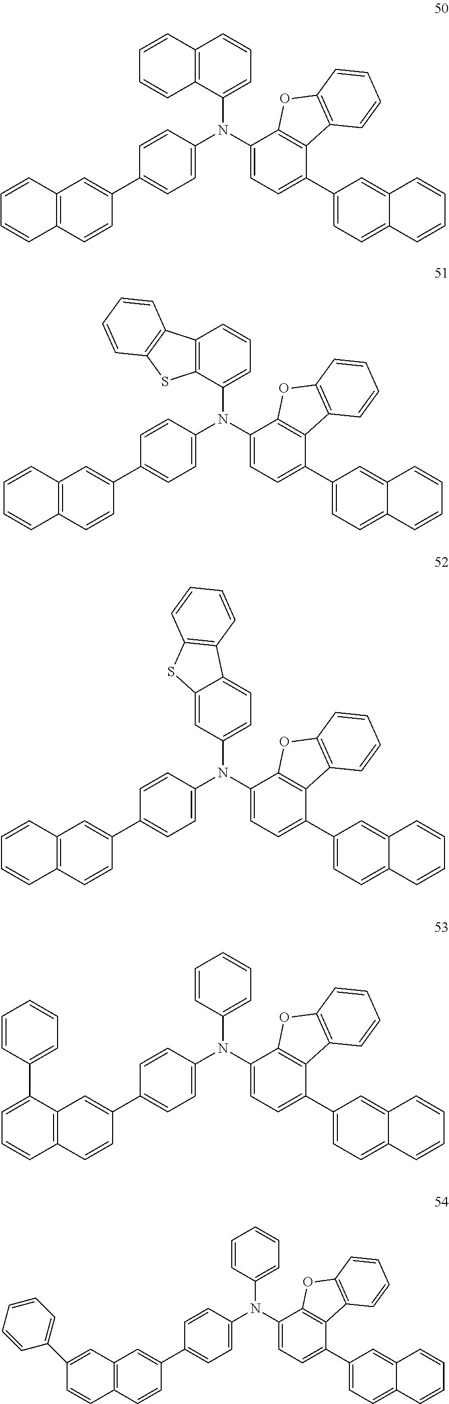 [SMILES: c1ccc2cc(-c3ccc(N(c4cccc5ccccc45)c4ccc(-c5ccc6ccccc6c5)c5c4oc4ccccc45)cc3)ccc2c1 ; c1ccc2cc(-c3ccc(N(c4ccc(-c5ccc6ccccc6c5)c5c4oc4ccccc45)c4cccc5c4sc4ccccc45)cc3)ccc2c1 ; c1ccc2cc(-c3ccc(N(c4ccc5c(c4)sc4ccccc45)c4ccc(-c5ccc6ccccc6c5)c5c4oc4ccccc45)cc3)ccc2c1 ; c1ccc(-c2cccc3ccc(-c4ccc(N(c5ccccc5)c5ccc(-c6ccc7ccccc7c6)c6c5oc5ccccc56)cc4)cc23)cc1 ; c1ccc(-c2ccc3ccc(-c4ccc(N(c5ccccc5)c5ccc(-c6ccc7ccccc7c6)c6c5oc5ccccc56)cc4)cc3c2)cc1] 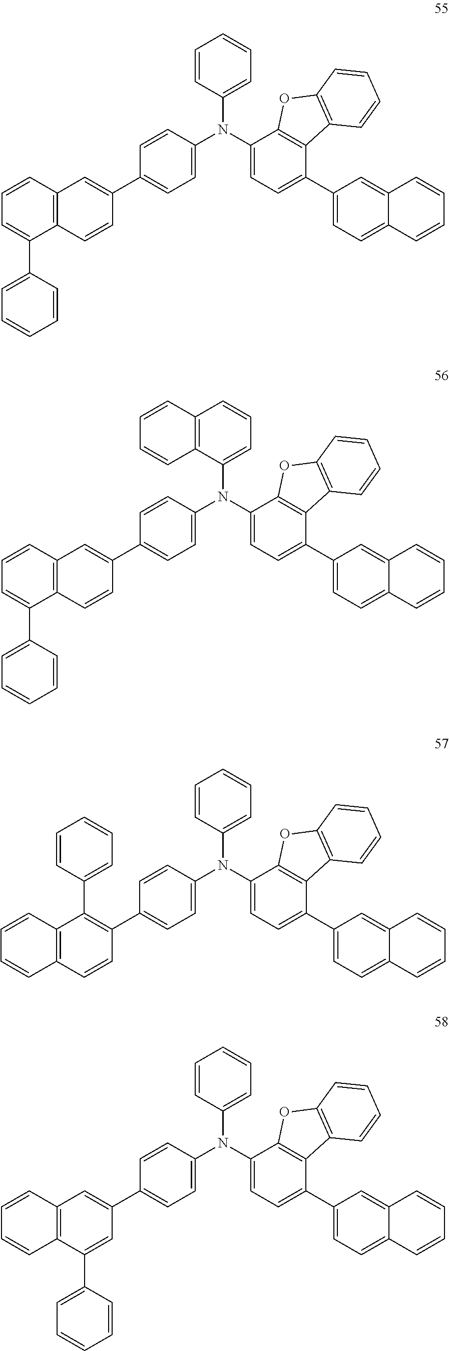 [SMILES: c1ccc(-c2cccc3cc(-c4ccc(N(c5ccccc5)c5ccc(-c6ccc7ccccc7c6)c6c5oc5ccccc56)cc4)ccc23)cc1 ; c1ccc(-c2cccc3cc(-c4ccc(N(c5cccc6ccccc56)c5ccc(-c6ccc7ccccc7c6)c6c5oc5ccccc56)cc4)ccc23)cc1 ; c1ccc(-c2c(-c3ccc(N(c4ccccc4)c4ccc(-c5ccc6ccccc6c5)c5c4oc4ccccc45)cc3)ccc3ccccc23)cc1 ; c1ccc(-c2cc(-c3ccc(N(c4ccccc4)c4ccc(-c5ccc6ccccc6c5)c5c4oc4ccccc45)cc3)cc3ccccc23)cc1] 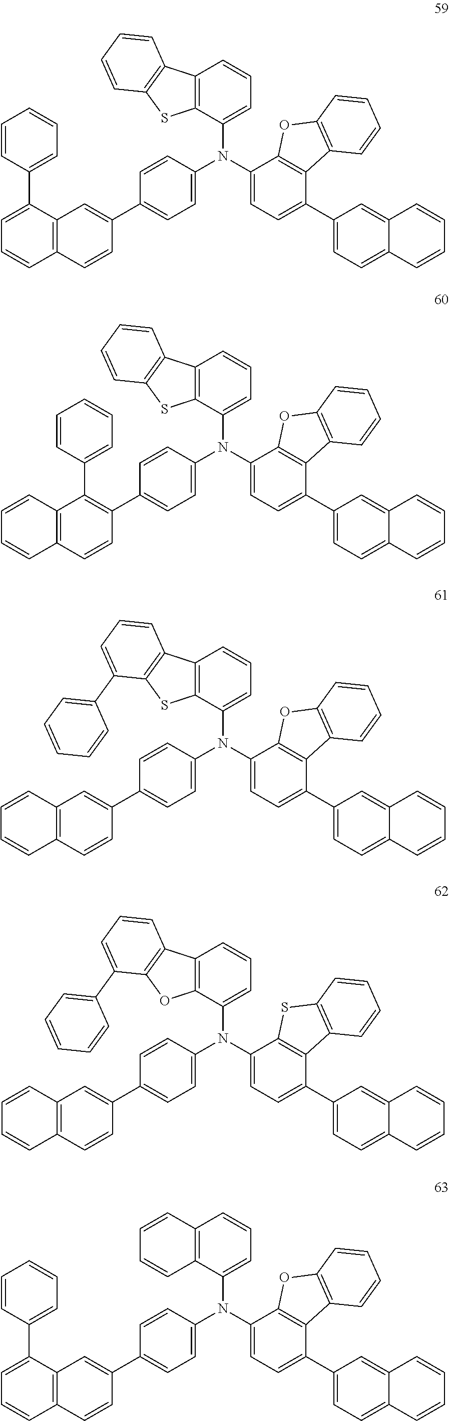 [SMILES: c1ccc(-c2cccc3ccc(-c4ccc(N(c5ccc(-c6ccc7ccccc7c6)c6c5oc5ccccc56)c5cccc6c5sc5ccccc56)cc4)cc23)cc1 ; c1ccc(-c2c(-c3ccc(N(c4ccc(-c5ccc6ccccc6c5)c5c4oc4ccccc45)c4cccc5c4sc4ccccc45)cc3)ccc3ccccc23)cc1 ; c1ccc(-c2cccc3c2sc2c(N(c4ccc(-c5ccc6ccccc6c5)cc4)c4ccc(-c5ccc6ccccc6c5)c5c4oc4ccccc45)cccc23)cc1 ; c1ccc(-c2cccc3c2oc2c(N(c4ccc(-c5ccc6ccccc6c5)cc4)c4ccc(-c5ccc6ccccc6c5)c5c4sc4ccccc45)cccc23)cc1 ; c1ccc(-c2cccc3ccc(-c4ccc(N(c5cccc6ccccc56)c5ccc(-c6ccc7ccccc7c6)c6c5oc5ccccc56)cc4)cc23)cc1] 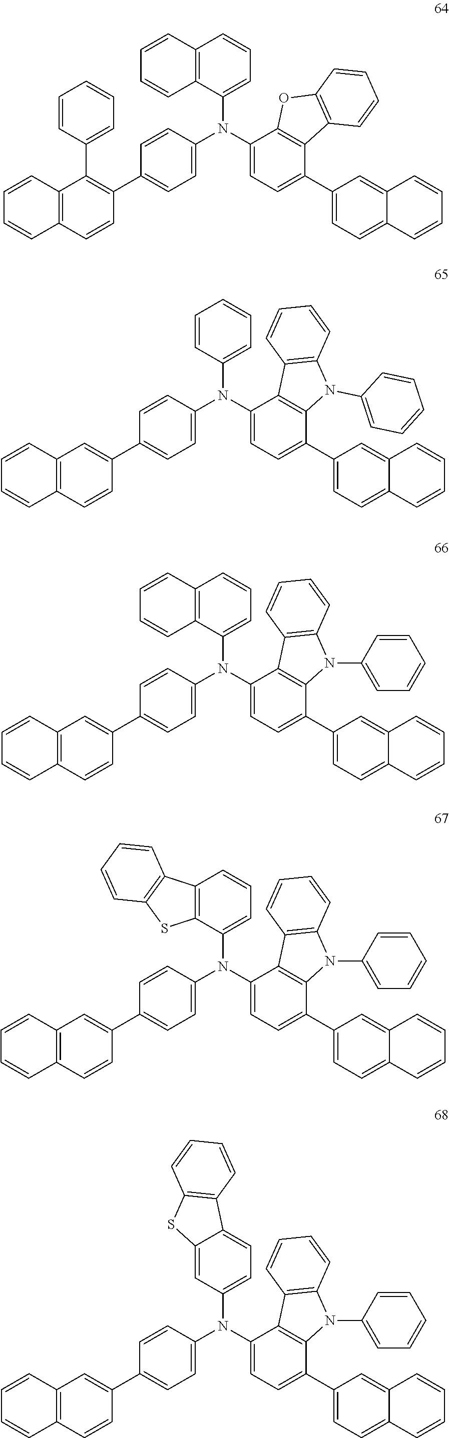 [SMILES: c1ccc(-c2c(-c3ccc(N(c4cccc5ccccc45)c4ccc(-c5ccc6ccccc6c5)c5c4oc4ccccc45)cc3)ccc3ccccc23)cc1 ; c1ccc(N(c2ccc(-c3ccc4ccccc4c3)cc2)c2ccc(-c3ccc4ccccc4c3)c3c2c2ccccc2n3-c2ccccc2)cc1 ; c1ccc(-n2c3ccccc3c3c(N(c4ccc(-c5ccc6ccccc6c5)cc4)c4cccc5ccccc45)ccc(-c4ccc5ccccc5c4)c32)cc1 ; c1ccc(-n2c3ccccc3c3c(N(c4ccc(-c5ccc6ccccc6c5)cc4)c4cccc5c4sc4ccccc45)ccc(-c4ccc5ccccc5c4)c32)cc1 ; c1ccc(-n2c3ccccc3c3c(N(c4ccc(-c5ccc6ccccc6c5)cc4)c4ccc5c(c4)sc4ccccc45)ccc(-c4ccc5ccccc5c4)c32)cc1] 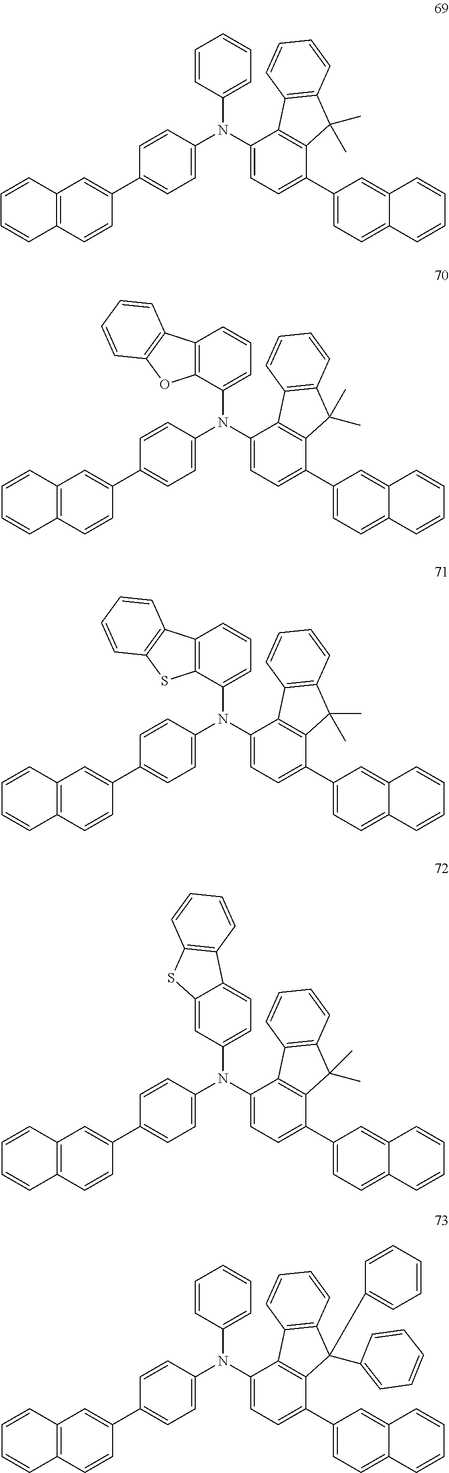 [SMILES: CC1(C)c2ccccc2-c2c(N(c3ccccc3)c3ccc(-c4ccc5ccccc5c4)cc3)ccc(-c3ccc4ccccc4c3)c21 ; CC1(C)c2ccccc2-c2c(N(c3ccc(-c4ccc5ccccc5c4)cc3)c3cccc4c3oc3ccccc34)ccc(-c3ccc4ccccc4c3)c21 ; CC1(C)c2ccccc2-c2c(N(c3ccc(-c4ccc5ccccc5c4)cc3)c3cccc4c3sc3ccccc34)ccc(-c3ccc4ccccc4c3)c21 ; CC1(C)c2ccccc2-c2c(N(c3ccc(-c4ccc5ccccc5c4)cc3)c3ccc4c(c3)sc3ccccc34)ccc(-c3ccc4ccccc4c3)c21 ; c1ccc(N(c2ccc(-c3ccc4ccccc4c3)cc2)c2ccc(-c3ccc4ccccc4c3)c3c2-c2ccccc2C3(c2ccccc2)c2ccccc2)cc1] 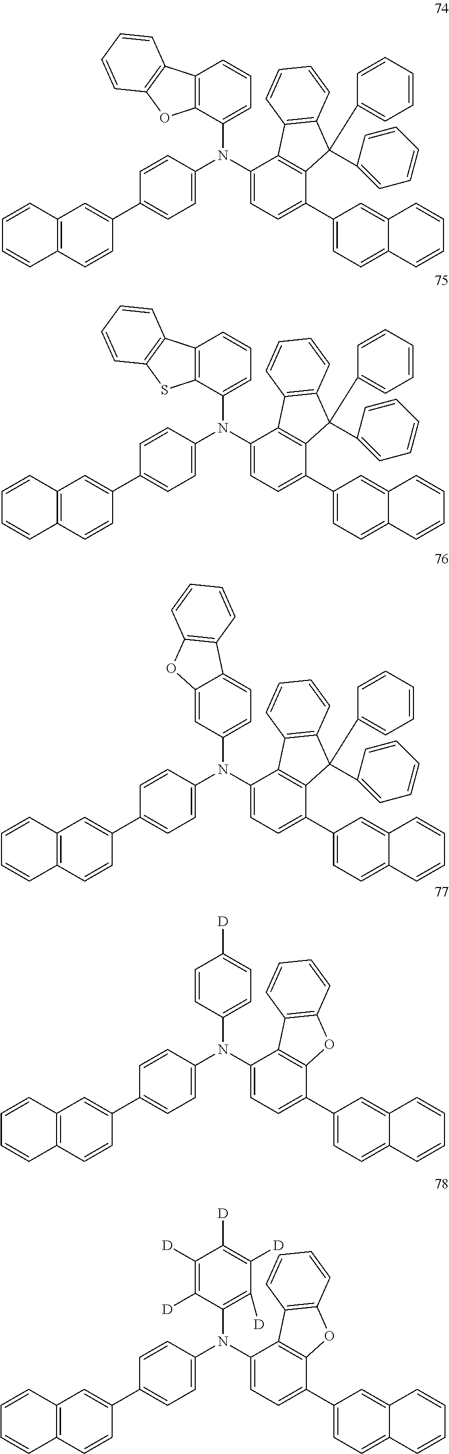 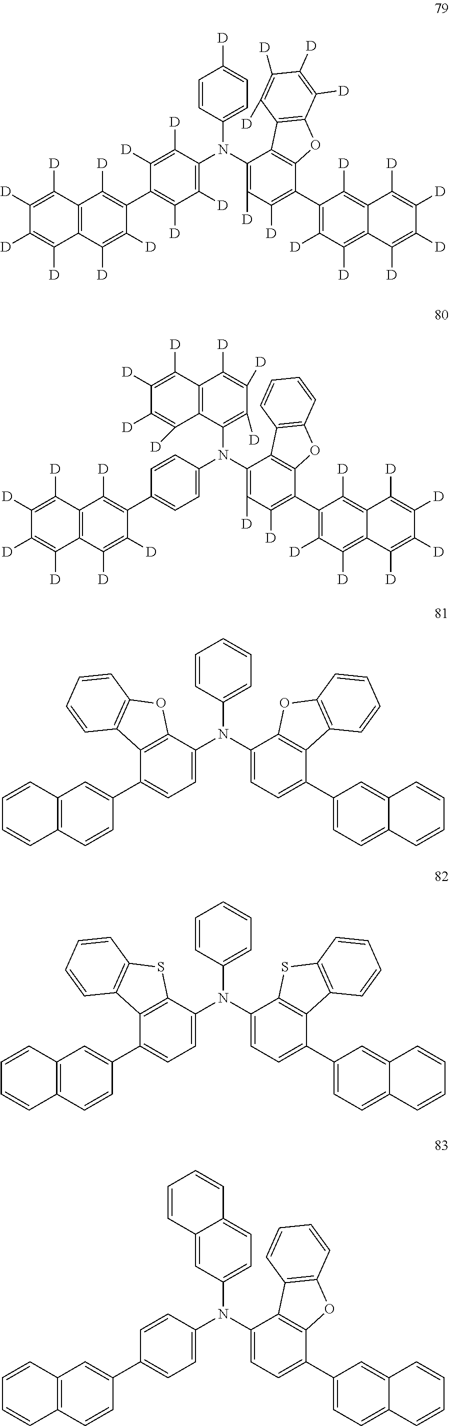 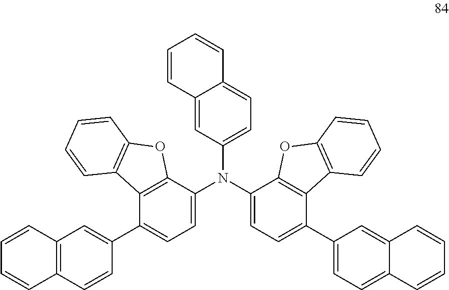 [SMILES: c1ccc2cc(-c3ccc(N(c4ccc5ccccc5c4)c4ccc(-c5ccc6ccccc6c5)c5c4oc4ccccc45)c4oc5ccccc5c34)ccc2c1]
wherein in Compound Group 1,
D indicates a deuterium atom.
